(19) United States
(12) Patent Application Publication
(10) Pub. No.: US 2010/0292282 A1
(43) Pub. Date: Nov. 18, 2010

(54) SYNTHESIS AND CRYSTALLINE FORMS OF CB-1 ANTAGONIST/INVERSE AGONIST

(76) Inventors: Kevin R. Campos, Berkeley Heights, NJ (US); Cheng-Yi Chen, Plainsboro, NJ (US); Jennifer R. Foley, Garwood, NJ (US); Michael Hillier, Libertyville, IL (US); Wendy S. Jen, Fanwood, NJ (US); Amude M. Kassim, Monmouth Junction, NJ (US); Artis Klapars, Edison, NJ (US); Andrey V. Peresypkin, Cambridge, MA (US); Thorsten Rosner, Berkeley Heights, NJ (US); Cecile G. Savarin, Newbury Park, CA (US); Matthew Tudge, Chatham, NJ (US); Narayan Variankaval, Plainsboro, NJ (US); Vicky Vydra, Tinton Falls, NJ (US); Dalian Zhao, Fanwood, NJ (US)

Correspondence Address: MERCK, P O BOX 2000, RAHWAY, NJ, 07065-0907, US

(21) Appl. No.: 12/681,797

(22) PCT Filed: Oct. 20, 2008
(86) PCT No.: PCT/US2008/011927

§ 371 Date: Jun. 14, 2010

Related U.S. Application Data
(60) Provisional application No. 61/000,151, filed on Oct. 24, 2007.

(51) Int. Cl. A61K 31/4245; C07D 413/10; C07D 271/113; C07C 255/53; A61P 3/04; A61P 3/10; A61P 25/28

(52) U.S. Cl. 514/364; 548/144; 558/423

(57) ABSTRACT

The present invention relates to a process for producing crystalline 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methyl-propyl]-5-fluorobenzonitrile, and novel salts, solvates, hydrates and polymorphs thereof.

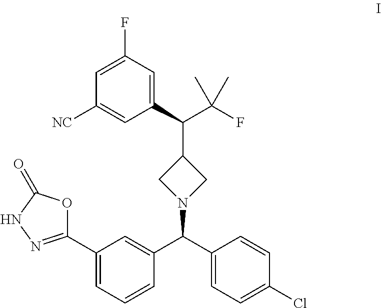

### BACKGROUND OF THE INVENTION

The compound of structural formula I was previously disclosed in US 2007/0123505, WO 2007/062193 and WO 2007/064566.

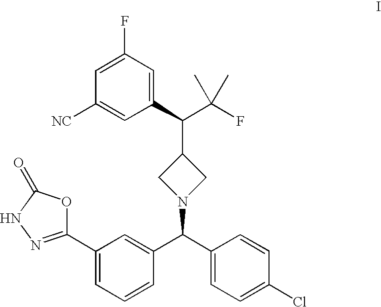

The compound of structural formula I, and its novel polymorphic forms, solvates, hydrates and salts, are CB1 modulators characterized as inverse agonists/antagonists useful as centrally acting drugs in the treatment of various diseases related to CB-1 modulation, including, but not limited to, psychosis, memory deficits, cognitive disorders, Alzheimer's disease, Huntington's disease, migraine, neuropathy, neuro-inflammatory disorders including multiple sclerosis and Guillain-1-Barre syndrome and the inflammatory sequelae of viral encephalitis, cerebral vascular accidents, and head trauma, anxiety disorders, stress, epilepsy, Parkinson's disease, movement disorders, and schizophrenia. The compounds are also useful for the treatment of substance abuse disorders, the treatment of obesity or eating disorders, and complications associated therewith, including left ventricular hypertrophy, as well as the treatment of asthma, constipation, chronic intestinal pseudo-obstruction, and cirrhosis of the liver.

The invention describes a novel and efficient process for the synthesis of the potent CB-1 inverse agonist Compound I, which was previously prepared using a linear synthesis requiring the use of HF to install the fluorine group and requiring column chromatography to separate the diastereomers of Compound I. The synthesis of the present invention is convergent, provides a higher yield of product and provides crystalline intermediates, which is an advantage of this invention with regard to isolation and purification without the use of chromatography.

Reviews of Witting and Horner-Wadsworth-Emmons reactions are provided in Bonadies, F. et al., Tetrahedron Lett. 1994, 35, 20, 3383-3386; and Fukatsu K. et al., J. Med. Chem. 2002, 45, 4212-4221. Chiral phosphorous ligands for enantioselective hydrogenations are described in Tang, W. et al., Chem. Rev. 2003, 103, 3029. Methods for reducing β-carboxyesters are provided in Kastrinsky, D. B. et al. J. Org. Chem. 2004, 69, 2284; and Lewis, E. A. et al., Tetrahedron Lett. 2004, 45, 3059. Rhodium catalyzed additions of arylboronic acids to sulfinylimines is described in Weix, D. et al., J. Am. Chem. Soc. 127(4), 1092-1093 (2005); and Bolshan, Y. et al., Org. Lett. 7 (8), 1481-1484 (2005). Metal-halogen exchange reactions utilizing n-BuLi/n-Bu₂Mg are described in Kitagawa, K. et al., Angew. Chem., Int. Ed. 39 (14), 2481-2483 (2000). The preparation of azetidines from diols via bis-alkylation is described in Hillier, M. C.; Chen, C-y., J. Org. Chem. 71, 7885-7887 (2006).

### SUMMARY OF THE INVENTION

This invention provides a novel and efficient process for producing 3-[(1S)-1-(1-{(S)-(4-chlorophenyl)[3-(5-oxo-4,5-dihydro-1,3,4-oxadiazol-2-yl) phenyl]methyl}azetidin-3-yl)-2-fluoro-2-methylpropyl]-5-fluorobenzonitrile of structural formula I from benzhydrylamine II and cyanodiol III.

This invention further provides eleven novel crystalline forms of 3-[(1S)-1-(1-{(S)-(4-chlorophenyl)[3-(5-oxo-4,5-dihydro-1,3,4-oxadiazol-2-yl) phenyl]methyl}azetidin-3-yl)-2-fluoro-2-methylpropyl]-5-fluorobenzonitrile that have been identified are designated as 1) free base anhydrous polymorphic Form I of Compound I; 2) free base toluene/heptane solvate polymorphic Form I, Type B of Compound I; 3) free base isopropyl acetate/methyl cyclohexane solvate polymorphic Form I, Type A of Compound I; and 4) HCl salt anhydrous polymorphic Forms A, B, C, D, E, F, G and H of Compound I. The crystalline forms of these free base and hydrochloric acid salt polymorphs are new and may have advantages in the preparation of pharmaceutical compositions of Compound I, such as ease of processing, handling and dosing. In particular, the anhydrous crystalline free base Form I of Compound I has improved physiochemical properties, such as lipid based solubility; good pK exposure; chemical and physical stability; purity; ease of purification and isolation; and formulation due to desirable crystal size, crystal surface area, and the lack of crystal aggregation that render it particularly suitable for the manufacture of pharmaceutical dosage forms. The novel HCl salt anhydrous polymorphic Form G of Compound I is the most thermodynamically stable crystalline HCl salt form of Compound I, however, forms A, B, F and H are more kinetically favored.

The present invention also relates to pharmaceutical formulations comprising the novel polymorphs and salts of compound I as active pharmaceutical ingredients, as well as methods for using them as CB-1 inverse agonists/antagonists in the treatment of CB-1 related disorders.

### BRIEF DESCRIPTION OF THE DRAWINGS

FIG. 1 is the X-ray diffraction (XRPD) pattern for the anhydrous free base polymorphic Form I of Compound I.

FIG. 2 is the Thermogravimetry analysis (TGA) curve for the anhydrous free base polymorphic Form I of Compound I.

FIG. 3 is the Differential scanning calorimetry (DSC) curve for the anhydrous free base polymorphic Form I of Compound I.

FIG. 4 is the X-ray diffraction (XRPD) pattern for anhydrous HCl salt polymorphic Form A of Compound I.

FIG. 5 is the Differential scanning calorimetry (DSC) curve for anhydrous HCl salt polymorphic Form A of Compound I.

FIG. 6 is the X-ray diffraction (XRPD) pattern for anhydrous HCl salt polymorphic Form B of Compound I.

FIG. 7 is the Differential scanning calorimetry (DSC) curve for anhydrous HCl salt polymorphic Form B of Compound I.

FIG. 8 is the X-ray diffraction (XRPD) pattern for anhydrous HCl salt polymorphic Form G of Compound I.

FIG. 9 is the Differential scanning calorimetry (DSC) curve for anhydrous HCl salt polymorphic Form G of Compound I.

FIG. 10 is the X-ray diffraction (XRPD) pattern for the freebase isopropyl acetate/methylcyclohexane solvate polymorphic Form I Type A of Compound I.

FIG. 11 is the Differential scanning calorimetry (DSC) curve for the freebase isopropyl acetate/methylcyclohexane solvate polymorphic Form I Type A of Compound I.

FIG. 12 is the Thermogravimetry analysis (TGA) curve for the freebase isopropyl acetate/methylcyclohexane solvate polymorphic Form I Type A of Compound I.

FIG. 13 is the X-ray diffraction (XRPD) pattern for the freebase toluene/heptane solvate of polymorphic Form I Type B of Compound I.

FIG. 14 is the Differential scanning calorimetry (DSC) curve for the freebase toluene/heptane solvate of polymorphic Form I Type B of Compound I.

FIG. 15 is the X-ray diffraction (XRPD) pattern for HCl salt polymorphic Form C of Compound I.

FIG. 16 is the X-ray diffraction (XRPD) pattern for HCl salt polymorphic Form D of Compound I.

FIG. 17 is the Differential scanning calorimetry (DSC) curve for the HCl salt polymorphic Form D of Compound I.

FIG. 18 is the Thermogravimetry analysis (TGA) curve for the HCl salt polymorphic Form D of Compound I.

FIG. 19 is the X-ray diffraction (XRPD) pattern for anhydrous HCl salt polymorphic Form E of Compound I.

FIG. 20 is the X-ray diffraction (XRPD) pattern for HCl salt polymorphic Form F hydrate of Compound I.

FIG. 21 is the X-ray diffraction (XRPD) pattern for HCl salt polymorphic Form H of Compound I.

### DETAILED DESCRIPTION OF THE INVENTION

The present invention provides a process for the preparation of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile of structural formula I

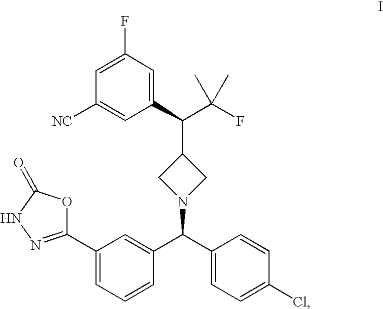

and crystalline polymorphs, solvates, hydrates and salts thereof.

As shown in the following General Scheme, Compound I can be prepared via the reaction of benzhydrylamine II with cyanodiol III to form the protected oxadiazole compound 20, followed by cleavage of the protecting group, P, to give compound I.

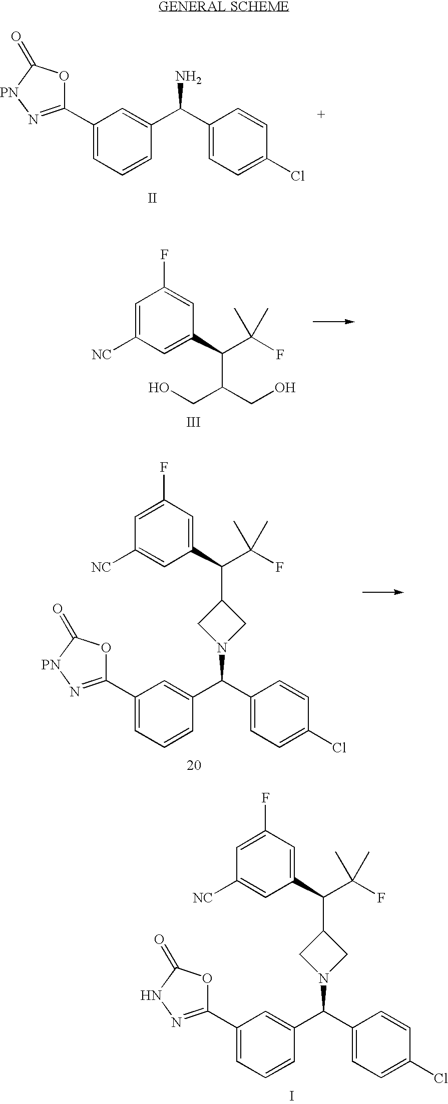

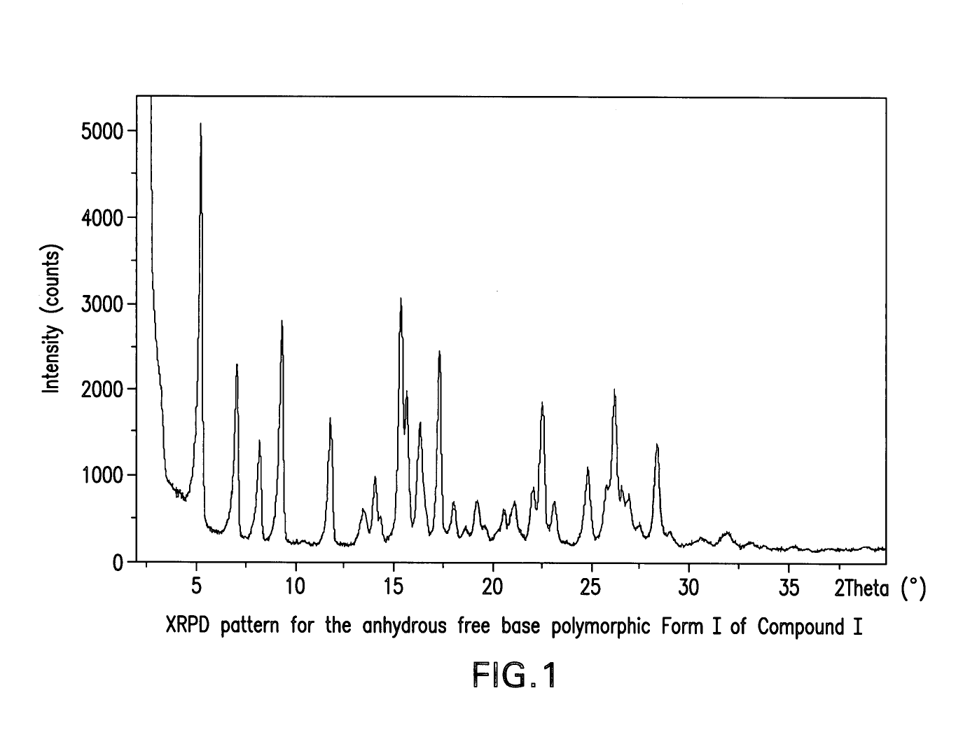

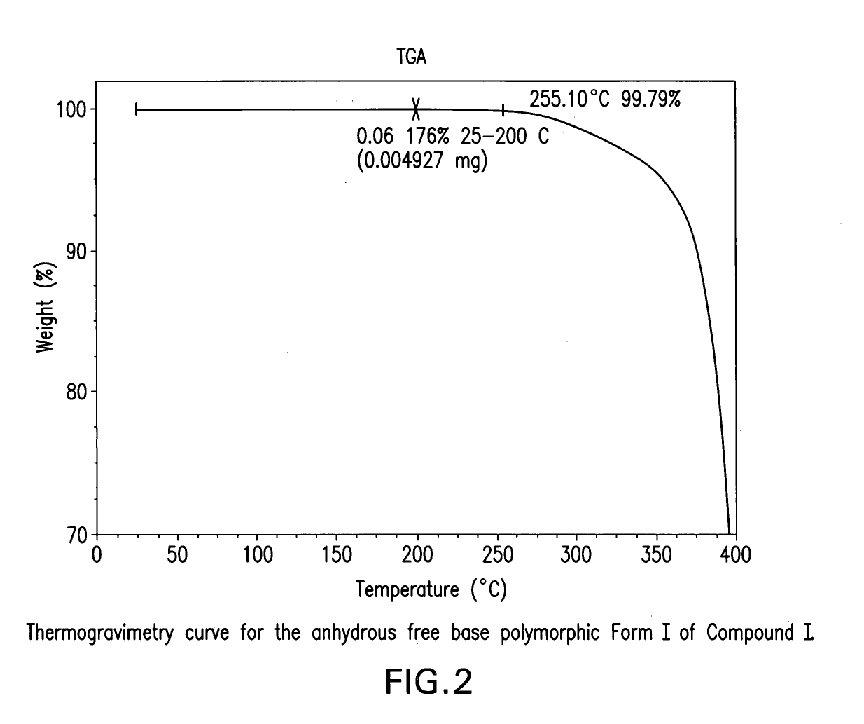

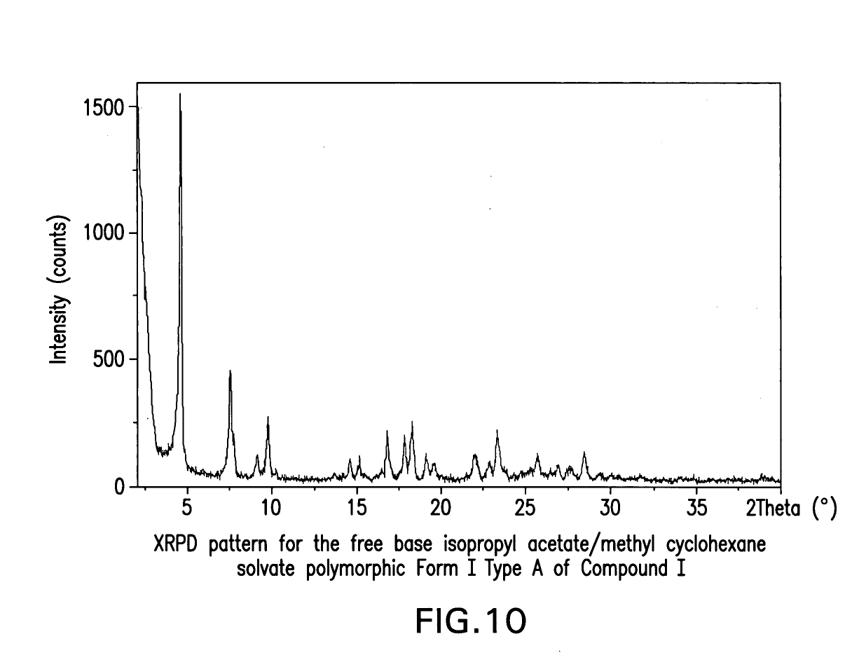

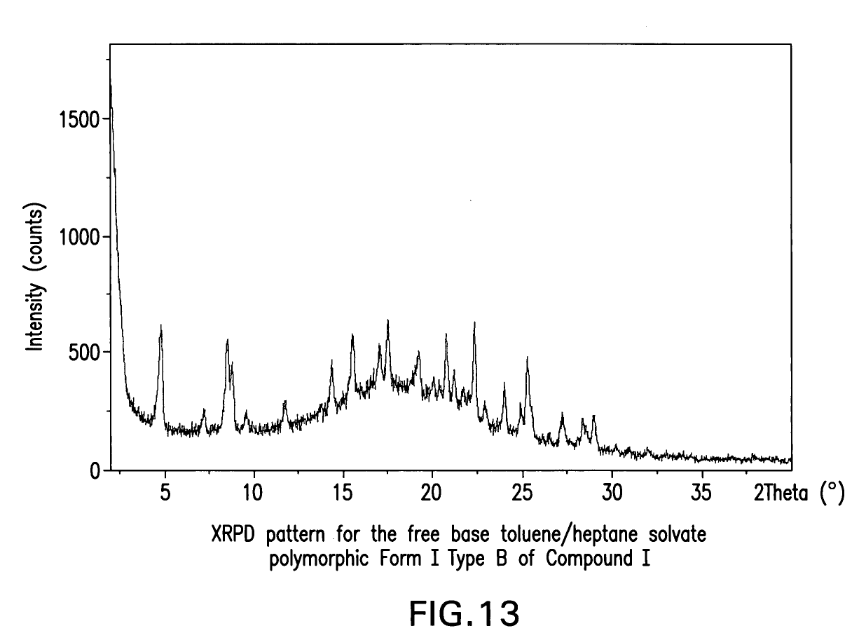

The free base of compound I has three known crystalline forms or polymorphs denoted as anhydrous free base polymorphic Form I, free base isopropyl acetate/methyl cyclohexane solvate polymorphic Form I type A, and free base toluene/heptane solvate polymorphic Form I type B. The X-ray powder diffraction (XPRD) patterns for the three free base crystalline forms of Compound I are shown in FIG. 1 (Form I), FIG. 10 (freebase isopropyl acetate/methyl cyclohexane solvate Form I Type A) and FIG. 13 (freebase toluene/heptane solvate Form I, Type B). The thermogravimetric analysis (TGA) curve in FIG. 2 was obtained on anhydrous free base polymorphic Form I of Compound I, under nitrogen flow at a heating rate of 10° C./minute, showed <0.1% weight loss from room temperature up to melting.

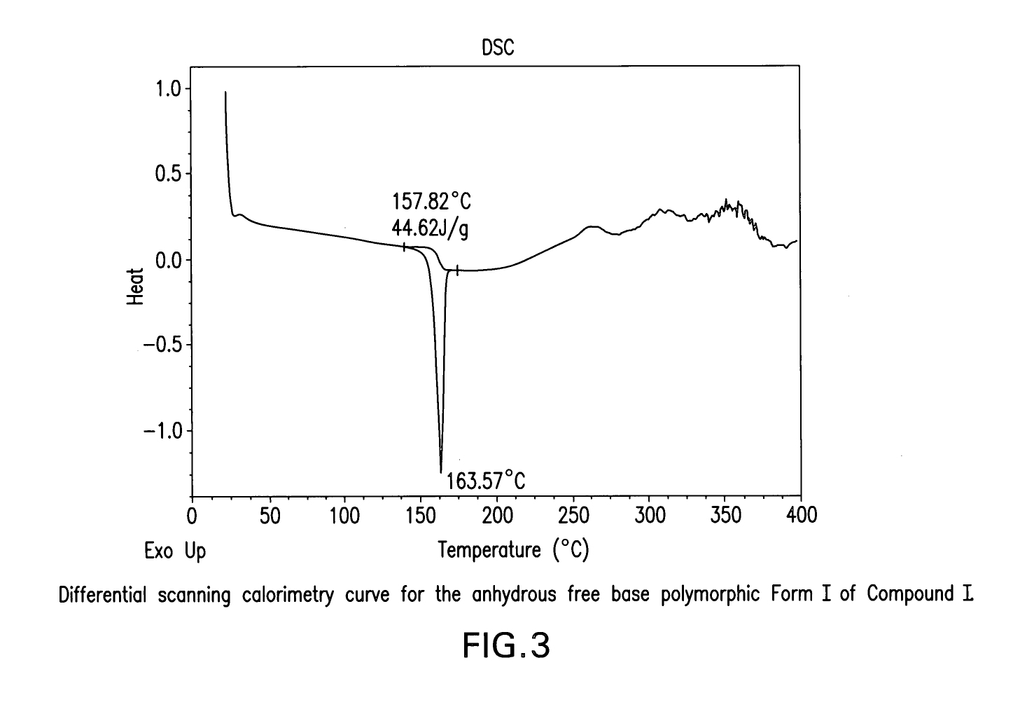

The DSC curve in FIG. 3 for anhydrous free base polymorphic Form I of Compound I is characterized by one endotherms with an extrapolated onset temperature of 157.8° C., a peak temperature of 163.6° C., and an associated heat of 44.6 J/g.

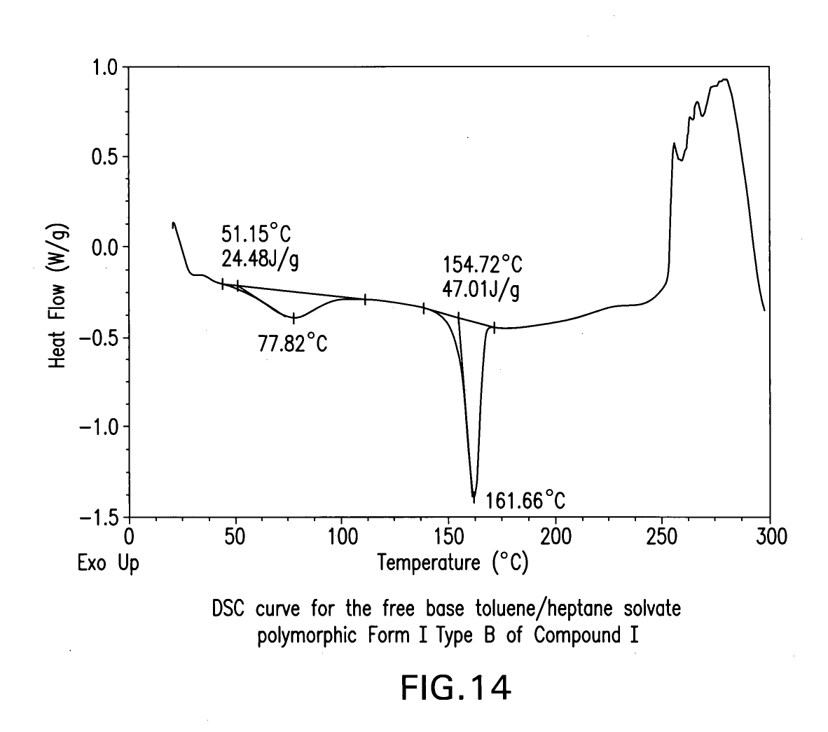

FIGS. 10, 11 and 12 show the X-ray diffraction pattern, TGA curve and DSC curve of the anhydrous freebase isopropyl acetate/methylcyclohexane solvate of Form I Type A of compound I. FIGS. 13 and 14 show the X-ray diffraction pattern and DSC curve of the freebase toluene/heptane solvate polymorphic Form I Type B of Compound I.

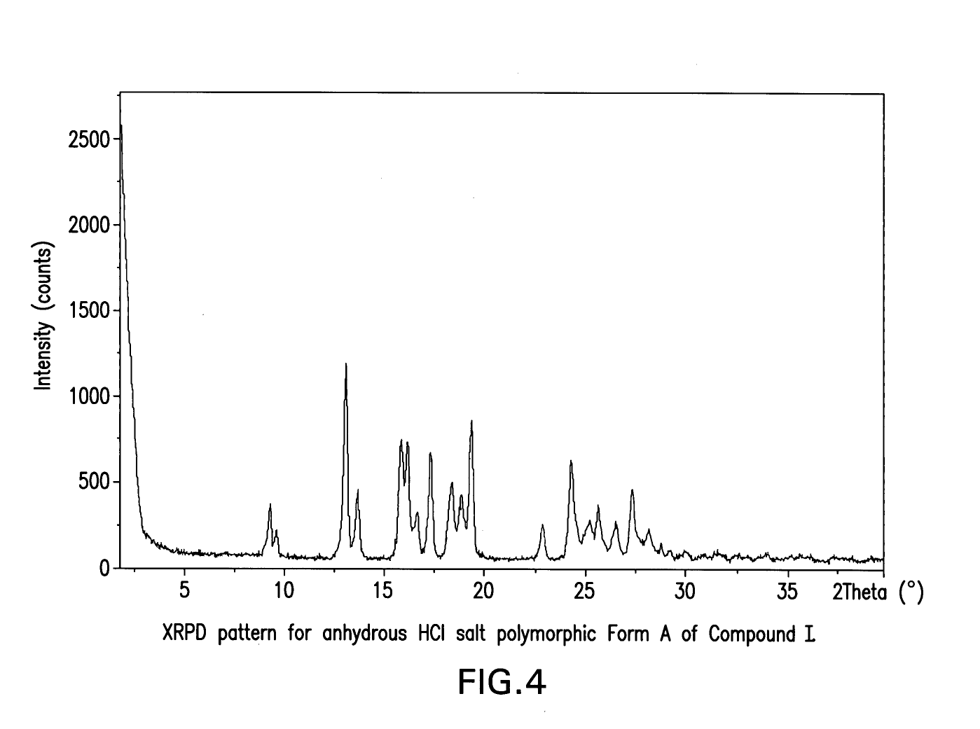

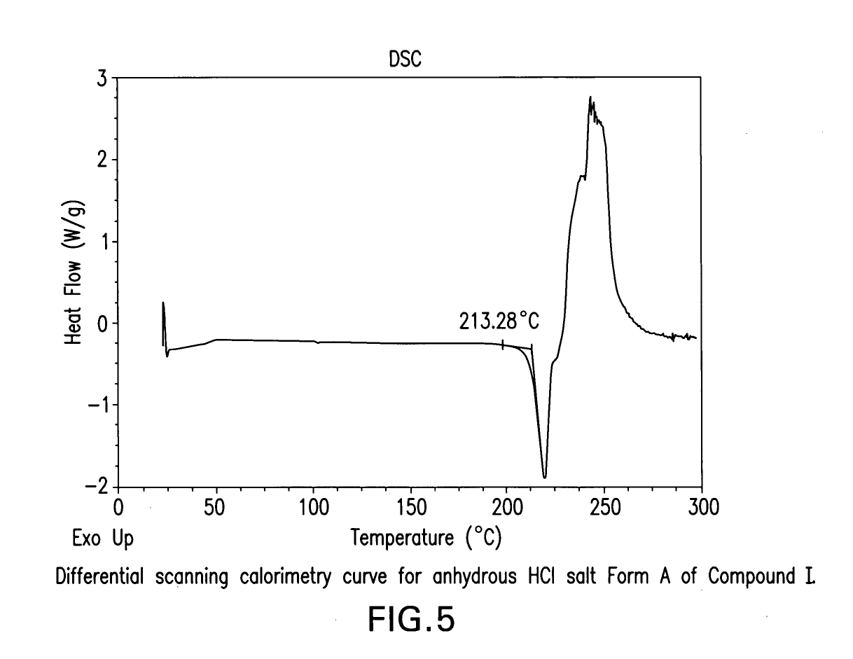

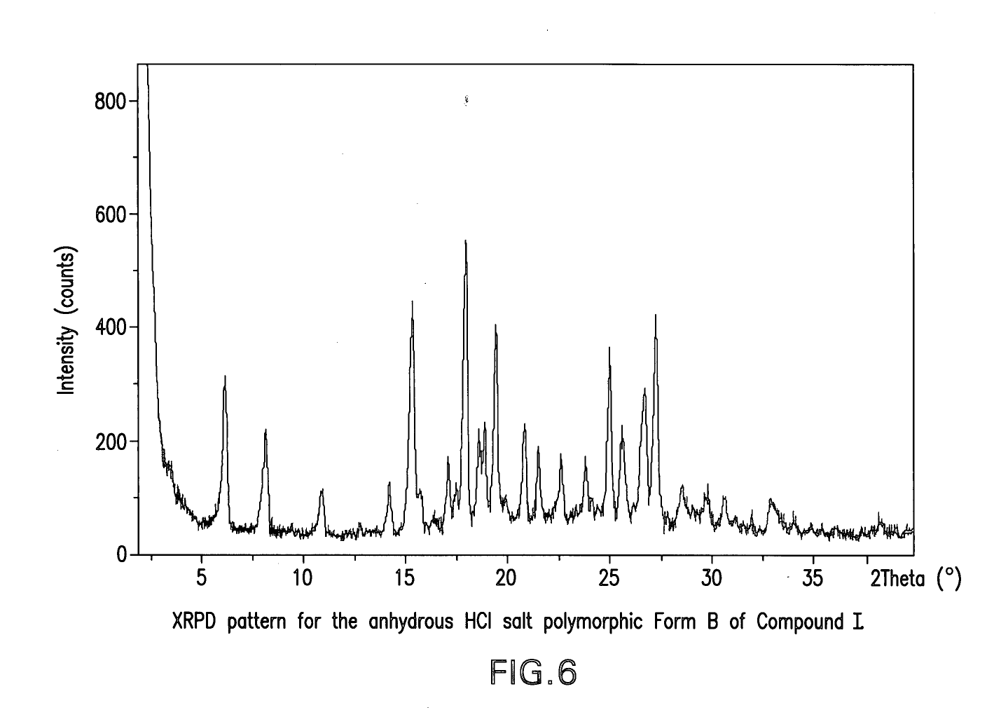

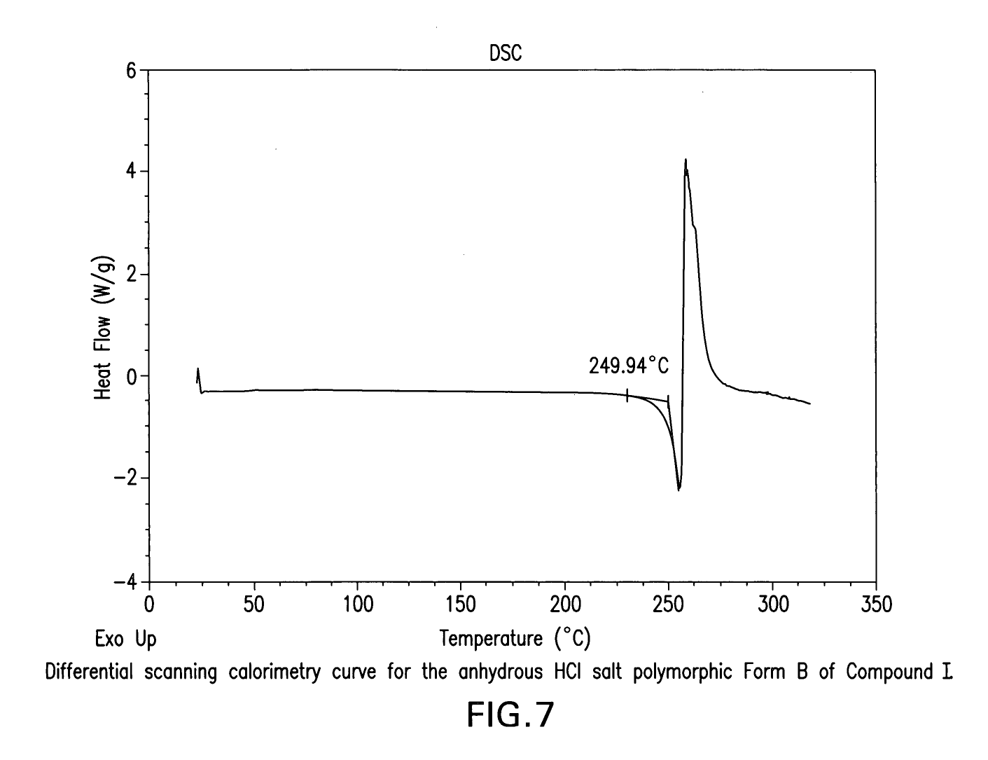

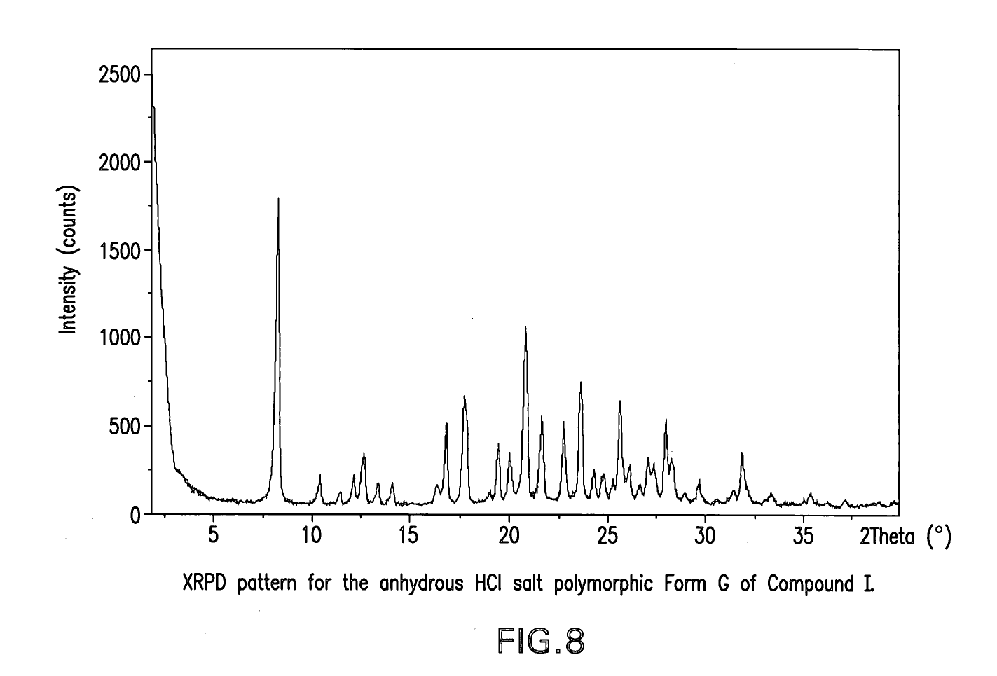

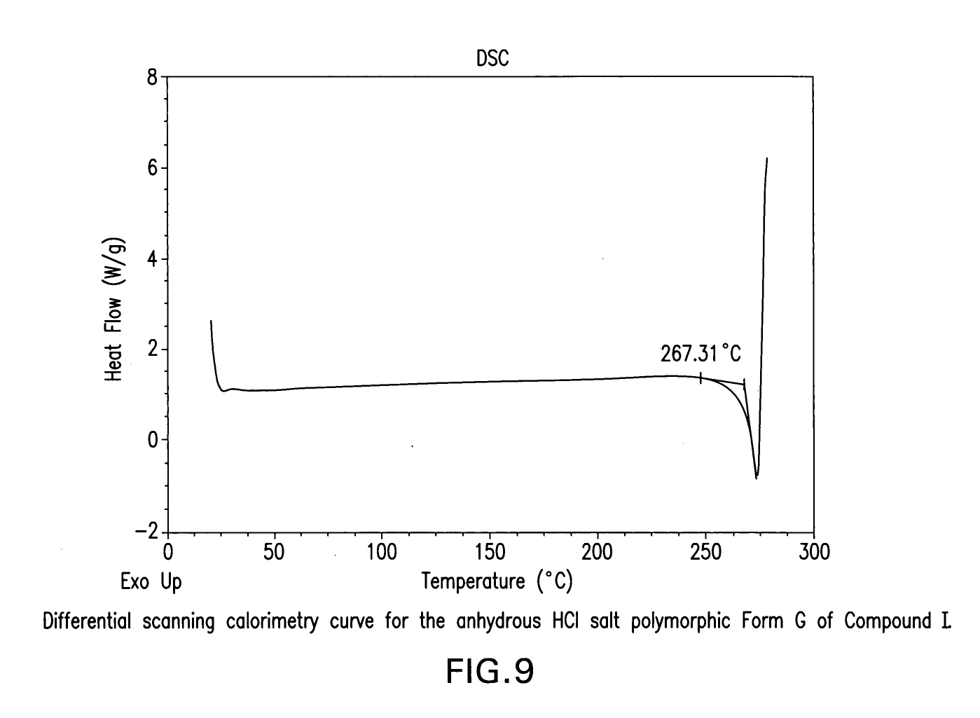

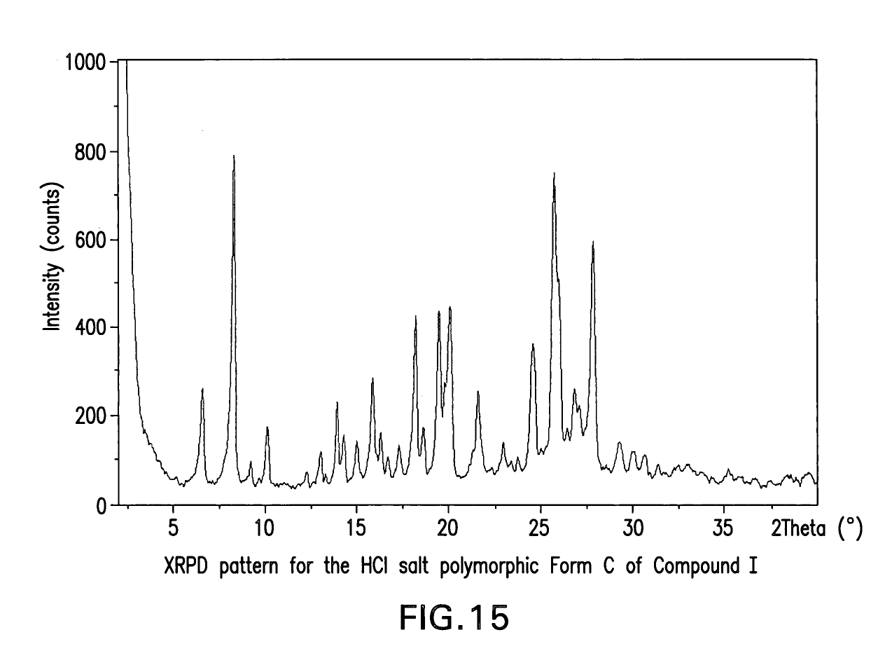

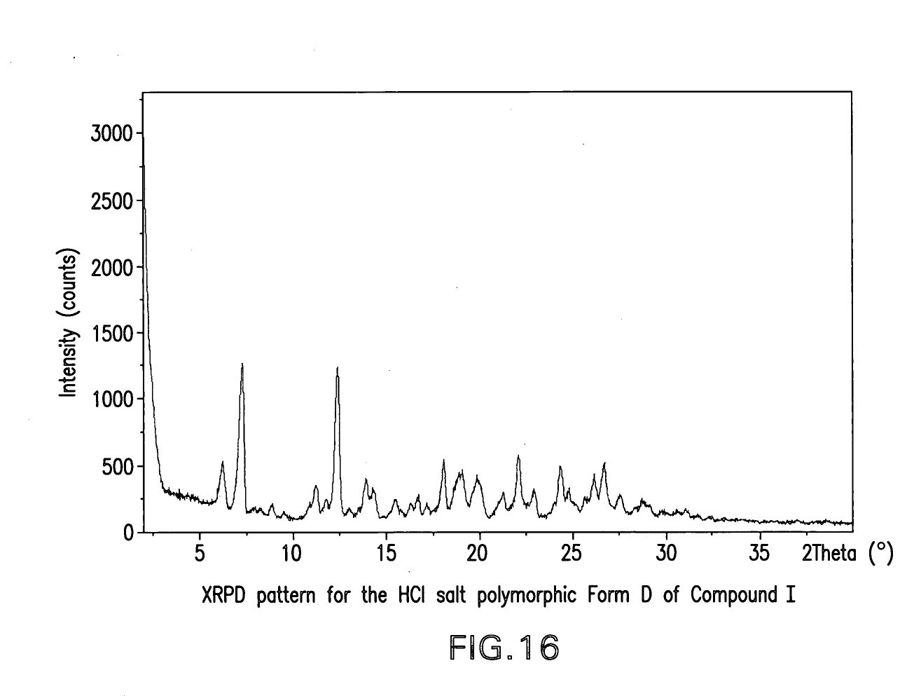

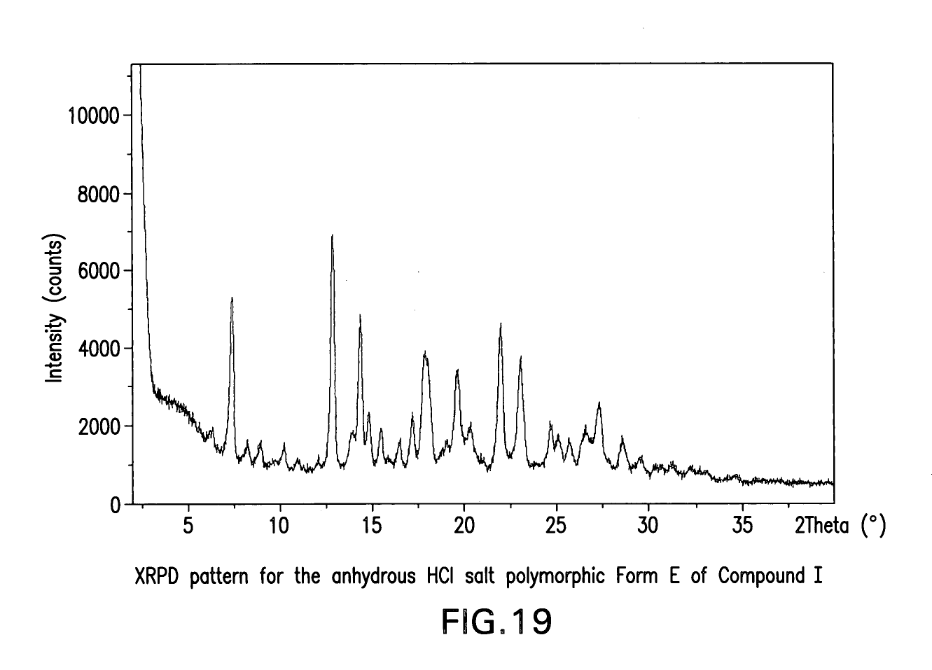

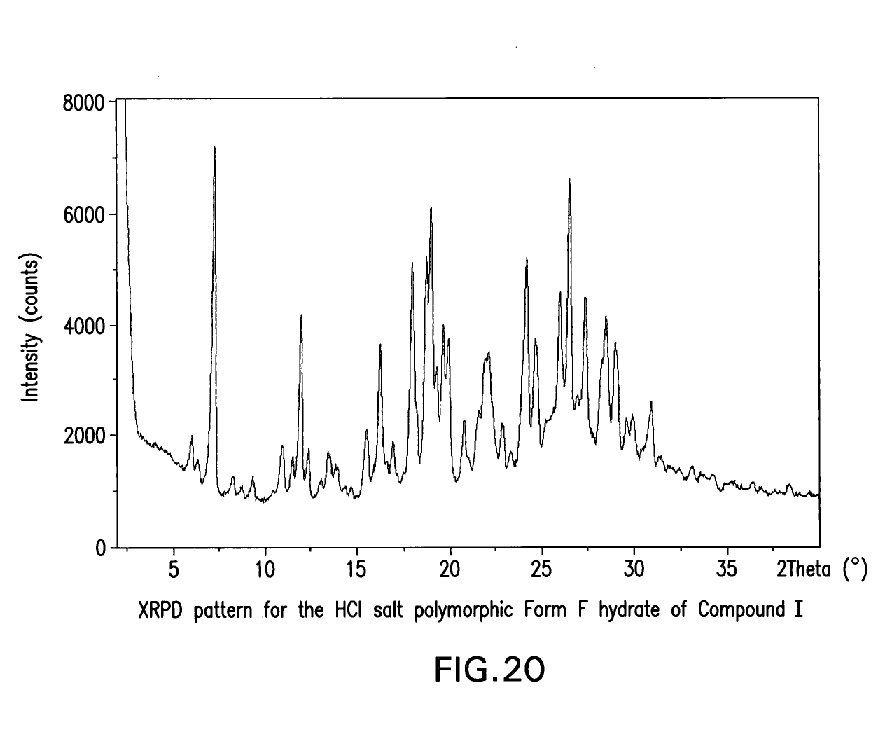

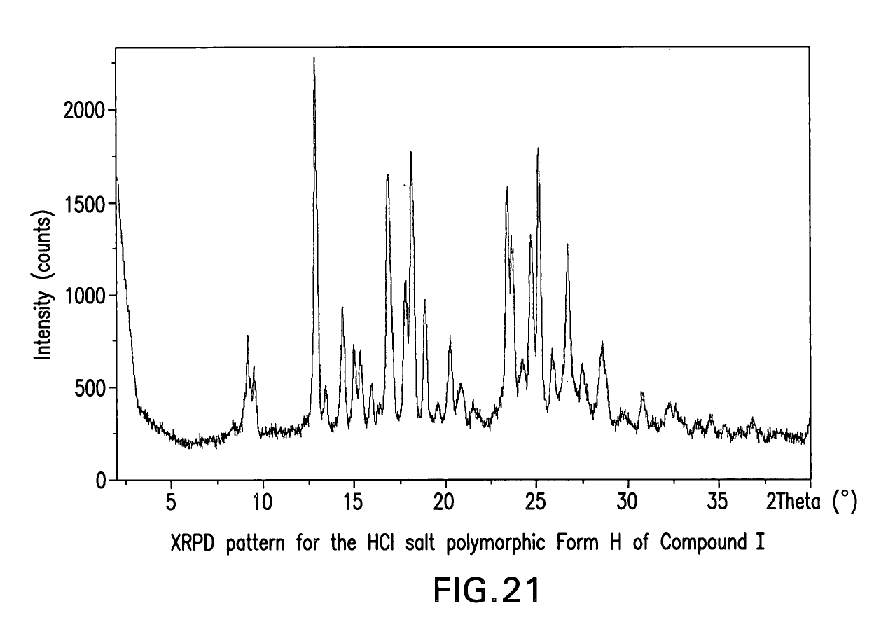

Compound I may further be converted to a hydrochloric acid salt as described below. FIGS. 4 and 5 show the X-ray diffraction pattern and DSC curve of the hydrochloric acid salt Form A of Compound I. FIGS. 6 and 7 show the X-ray diffraction pattern and DSC curve of the hydrochloric acid salt Form B of Compound I. FIGS. 8 and 9 show the X-ray diffraction pattern and DSC curve of the hydrochloric acid salt From G of Compound I. FIG. 15 shows the X-ray diffraction pattern for the hydrochloric acid salt Form C of Compound I. FIGS. 16, 17 and 18 show the X-ray diffraction pattern, TGA curve and DSC curve of the hydrochloric acid salt Form D of Compound I. FIG. 19 shows the X-ray diffraction pattern for the hydrochloric acid salt Form E of Compound I. FIG. 20 shows the X-ray diffraction pattern for the hydrochloric acid salt Form F of Compound I. Finally, FIG. 21 shows the X-ray diffraction pattern for the hydrochloric acid salt Form H of Compound I.

One embodiment of the present invention provides a process for preparing a compound of formula I, or a salt, hydrate or polymorph thereof,

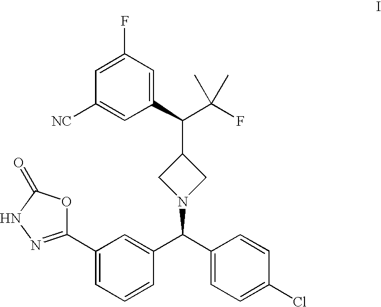

comprising the steps of:

A) coupling a compound of formula II wherein P is a protecting group, or a salt thereof,

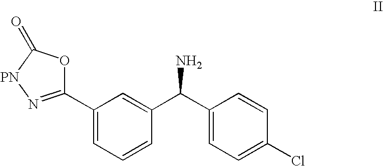

with a compound of formula III,

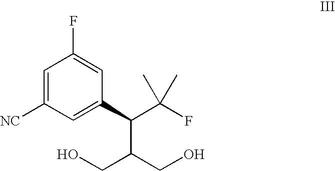

by converting the alcohol groups of compound III into leaving groups, followed by treatment with a hindered amine base; and

B) removal of the protecting group P.

In a class of this embodiment, the protecting group P of Step A and B is selected from Boc and CBZ.

In another class of this embodiment, the leaving groups of Step A are selected from triflates, tosylates, nosylates, and mesylates. In another class of this embodiment, the leaving groups of Step A are triflates and compound III is treated with triflic anhydride to form a di-triflate intermediate. In another class of this embodiment, the hindered amine base of Step A is selected from: diisopropyl ethyl amine, triethylamine, triisopropylamine and dicyclohexylamine.

In another class of this embodiment, the hindered amine base of Step A is diisopropyl ethyl amine. In another class of this embodiment, the reaction of Step A is run in acetonitrile.

In another class of this embodiment, the protecting group P is CBZ. In another class of this embodiment, the protecting group P is CBZ, and the CBZ protecting group in Step B is removed by hydrogenation.

In another class of this embodiment, the protecting group P is Boc. In another class of this embodiment, the protecting group is Boc and the Boc protecting group in Step B is removed using an acid. In a subclass of this subclass, the acid is selected from: HCl, H₂SO₄, H₃PO₄ and TFA. In another subclass of this subclass, the acid is HCl. In another subclass of this subclass, the acid is HCl in isopropanol. In another class of this embodiment, Step B is run in a solvent selected from: isopropyl acetate, isopropanol, methylene chloride and THF. In a subclass of this class, the solvent of Step B is isopropyl acetate.

In a class of this embodiment, the process further comprises isolating the compound of formula I. In a subclass of this class, the compound of formula I is isolated by recrystallizing from toluene/heptane.

Another embodiment of the present invention provides a process for preparing a compound of formula I, or a salt, solvate, hydrate or polymorph thereof,

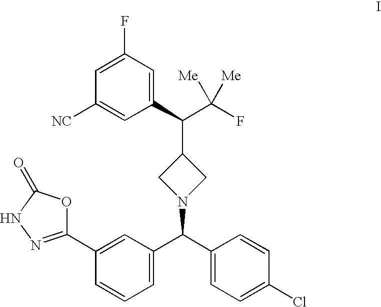

comprising removing the protecting group P of the compound of formula 20

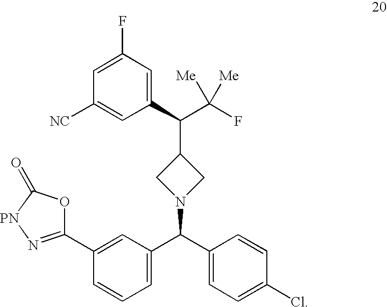

In a class of this embodiment, the protecting group P of Step A and B is selected from Boc and CBZ.

In another class of this embodiment, the protecting group P is CBZ. In another class of this embodiment, the protecting group P is CBZ, and the CBZ protecting group in Step B is removed by hydrogenation.

In another class of this embodiment, the protecting group P is Boc. In another class of this embodiment, the protecting group is Boc and the Boc protecting group in Step B is removed using an acid. In a subclass of this subclass, the acid is selected from: HCl, H₂SO₄, H₃PO₄ and TFA. In another subclass of this subclass, the acid is HCl. In another subclass of this subclass, the acid is HCl in isopropanol. In another class of this embodiment, Step B is run in a solvent selected from: isopropyl acetate, isopropanol, methylene chloride and THF. In a subclass of this class, the solvent of Step B is isopropyl acetate.

In a class of this embodiment, the process further comprises isolating the compound of formula I. In a subclass of this class, the compound of formula I is isolated by recrystallizing from toluene/heptane.

Another embodiment of the present invention provides a process for preparing a compound of formula I, or a salt, solvate, hydrate or polymorph thereof,

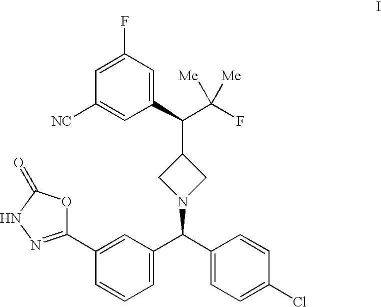

comprising removing the Boc protecting group of the compound of formula 20

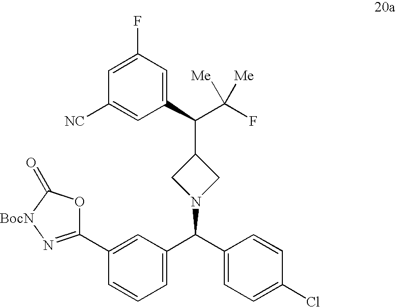

using an acid in a solvent.

In a class of this embodiment, the Boc protecting group is removed using an acid. In a subclass of this class, the acid is selected from: HCl, H₂SO₄, H₃PO₄ and TFA. In another subclass of this class, the acid is HCl. In another subclass of this class, the acid is HCl in isopropanol. In another class of this embodiment, the deprotection reaction is run in a solvent selected from: isopropyl acetate, isopropanol, methylene chloride and THF. In a subclass of this class, the solvent is isopropyl acetate.

In a class of this embodiment, the process further comprises isolating the compound of formula I. In a subclass of this class, the compound of formula I is isolated by recrystallizing from toluene/heptane.

Another embodiment of the present invention provides a process for preparing a compound of formula 20, wherein P is a protecting group,

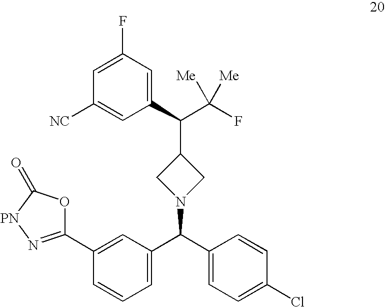

comprising the steps of coupling a compound of formula II, wherein P is a protecting group, or a salt thereof,

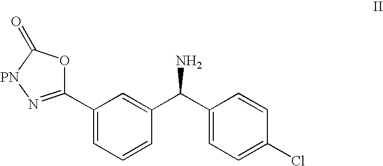

with a compound of formula III,

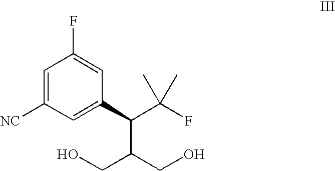

by converting the alcohol groups of compound III into leaving groups, followed by treatment with a hindered amine base.

In a class of this embodiment, the protecting group P is selected from Boc and CBZ. In another class of this embodiment, the leaving groups are selected from triflates, tosylates, nosylates, and mesylates. In another class of this embodiment, the leaving groups are triflates and compound III is treated with triflic anhydride to form a di-triflate intermediate. In another class of this embodiment, the hindered amine base is selected from: diisopropyl ethyl amine, triethylamine, triisopropylamine and dicyclohexylamine. In another class of this embodiment, the hindered amine base is diisopropyl ethyl amine. In another class of this embodiment, the reaction is run in acetonitrile.

In another class of this embodiment, the process further comprises isolating the compound of formula 20. In a subclass of this class, the compound of formula 20 is isolated by recrystallizing from isopropyl acetate, dicholoromethane, acetonitrile, heptane, or a mixture thereof. In another subclass of this class, the compound of formula 20 is isolated by recrystallizing from isopropyl acetate and heptane.

Another embodiment of the present invention provides a process for preparing a compound of formula II wherein P is a protecting group, or a salt thereof,

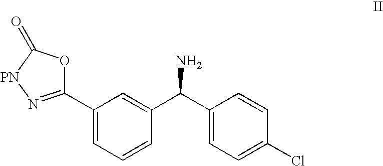

comprising the steps of:

(A) preparing a hydrazide of formula 3

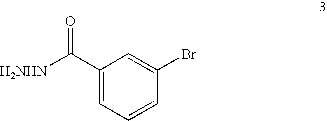

by treatment of a compound of formula 1

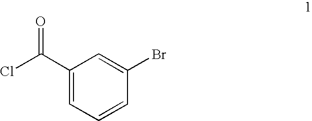

with a base, followed by treatment with hydrazine;

(B) forming an oxadiazole of formula 4

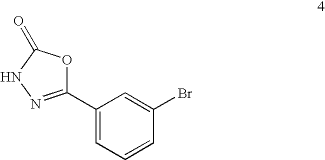

by treating the hydrazide of formula 3 with a coupling agent;

(C) preparing an aldehyde of formula 5

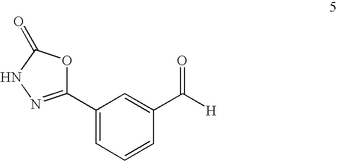

by treatment of the oxadiazole of formula 4 with an alkyl magnesium compound, followed by treatment with an alkyl lithium compound and DMF;
(D) preparing a N-tert-butyl sulfinyl imine of formula 6

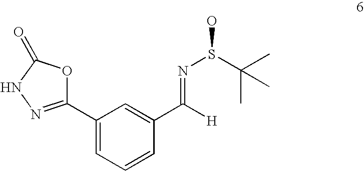

by treating the aldehyde of formula 5 with (S)-tert-butyl sulfinamide in the presence of a catalyst;
(E) forming a protected oxadiazole compound of formula 7, wherein P is a protecting group,

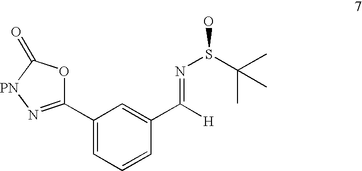

by adding a protecting group P to the oxadiazole nitrogen of the N-tert-butyl sulfinyl imine of formula 6;
(F) forming a N-tert-butyl sulfinyl amine of formula 8, wherein P is a protecting group,

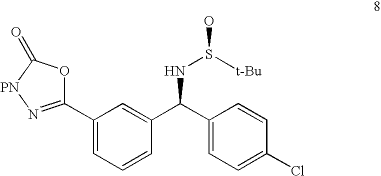

by treating the compound of formula 7 with boroxine 10 in the presence of a rhodium catalyst and a ligand; and
(G) forming a compound of formula II, wherein P is a protecting group,

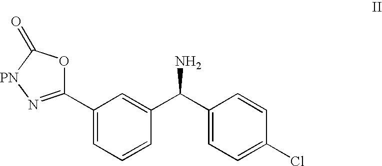

by cleaving the tert-butyl sulfoxide group of the compound of formula 8.

In a class of this embodiment, the base of Step A is selected from: DABCO, triethyl amine, and diisopropyl ethyl amine. In a subclass of this class, the base of Step A is DABCO. In another class of this embodiment, the reaction of Step A is run in methanol. In another class of this embodiment, the reaction of Step A is run between 50 to 55° C. In another class of this embodiment, the hydrazine of Step A is 64% hydrazine. In another class of this embodiment, the reaction of Step A is run with DABCO in methanol, followed by treatment with 64% hydrazine. In another class of this embodiment, the process further comprises isolating the hydrazide of formula 3. In a subclass of this class, the hydrazide of formula 3 is a solid. In yet another class of this embodiment, the process further comprises working up the reaction of Step A and using the hydrazide of formula 3 in a solution for Step B.

In another class of this embodiment, the coupling agent of Step B is selected from CDI, triphosgene and phosgene. In another class of this embodiment, the coupling agent of Step B is CDI. In another class of this embodiment, the reaction of Step B is run in an aprotic solvent. In a subclass of this class, the aprotic solvent is THF, toluene and ether. In another subclass of this class, the aprotic solvent is THF. In another class of this embodiment, the reaction of Step B is run at room temperature. In another class of this embodiment, the reaction of Step B is run with CDI in THF. In a subclass of this class, the reaction is run at room temperature. In another class of this embodiment, the process further comprises isolating the oxadiazole of formula 4 of Step B. In a subclass of this class, the oxadiazole of formula 4 is a solid. In yet another class of this embodiment, the process further comprises working up the reaction of Step B and using the oxadiazole of formula 4 in a solution for Step C.

In another class of this embodiment, the alkyl magnesium compound of Step C is selected from: dibutyl magnesium, dimethyl magnesium, diethyl magnesium, and dipropyl magnesium. In another class of this embodiment, the alkyl magnesium compound of Step C is di-n-butyl magnesium. In another class of this embodiment, the alkyl lithium compound of Step C is selected from: n-butyl lithium, sec-butyl lithium, tert-butyl lithium, and hexyl lithium. In another class of this embodiment, the alkyl lithium compound of Step C is n-butyl lithium. In another class of this embodiment, the reaction of Step C is run in an aprotic solvent. In a subclass of this class, the aprotic solvent of Step C is selected from: THF, toluene, MTBE, and diethyl ether. In another subclass of this class, the aprotic solvent of Step C is THF. In another class of this embodiment, the reaction of Step C is run at a temperature between about −20 to −78° C. In a subclass of this class, the reaction of Step C is run at a temperature between about −40 to −50° C. In another class of this embodiment, the reaction of Step C is run with di-n-butyl magnesium and n-butyl lithium in THF at a temperature between about −20 to −78° C. In a subclass of this class, the temperature is between about −40 to −50° C. In another class of this embodiment, the reaction of Step C is worked up with acid. In a subclass of this class, the acid of Step C is HCl or H₂SO₄. In a subclass of this class, the acid of Step C is HCl. In another class of this embodiment, the process further comprises isolating the aldehyde of formula 5. In a subclass of this class, the aldehyde of formula 5 is a solid. In yet another class of this embodiment, the process further comprises working up the reaction of Step C and using aldehyde of formula 5 in solution for Step D.

In another class of this embodiment, the catalyst of Step D is selected from: PPTS, KHSO₄, BF₃-etherate, Ti(OEt)₄, and TiCl₄/triethyl amine. In another class of this embodiment, the catalyst of Step D is PPTS. In another class of this embodiment, the reaction of Step D is run in a solvent selected from: toluene, methylene chloride and THF. In another class of this embodiment, the reaction of Step D is run in toluene. In another class of this embodiment, the reaction of Step D is run with PPTS in toluene. In a subclass of this class, the reaction of Step D is run at approximately 40° C. In another class of this embodiment, the process further comprises isolating the N-tert-butyl sulfinyl imine of formula 6 of Step D. In a subclass of this class, the N-tert-butyl sulfinyl imine of formula 6 is a solid. In yet another class of this embodiment, the process further comprises working up the reaction of Step D and using the N-tert-butyl sulfinyl imine of formula 6 in solution for Step E.

In a class of this embodiment, the protecting group P of the compound of formula 7 is a CBZ or Boc group. In another class of this embodiment, the compound of formula 7 is a N—CBZ protected oxadiazole wherein the protecting group P is CBZ. In another class of this embodiment, the protected oxadiazole compound of formula 7 is a N-Boc protected oxadiazole wherein the protecting group P is Boc. In a subclass of this class, the N-Boc protected oxadiazole of formula 7 is prepared by treating the N-tert-butyl sulfinyl imine of formula 6 with boc anhydride in the presence of a base. In a subclass of this subclass, the base is tertiary amine base. In another subclass of this subclass, the base is triethylamine. In another subclass of this class, the N-Boc protected oxadiazole is prepared by treating the N-tert-butyl sulfinyl imine of formula 6 with Boc anhydride in the presence of triethylamine in an aprotic solvent. In a subclass of this subclass, the aprotic solvent is THF. In another subclass of this subclass, the reaction of Step E is run at about 40° C. In another class of this embodiment, the compound of formula 7 is a N-Boc protected oxadiazole prepared by treating the N-tert-butyl sulfinyl imine of formula 6 with boc anhydride and triethyl amine in THF. In another class of this embodiment, the process further comprises isolating the protected oxadiazole compound of formula 7 of Step E. In a subclass of this class, the protected oxadiazole compound of formula 7 is a solid. In another class of this embodiment, the process further comprises working up the reaction of Step E and using the protected oxadiazole compound of formula 7 in solution for Step F.

In another class of this embodiment, the protecting group P of Step F is CBZ. In another class of this embodiment, the protecting group P of Step F is Boc. In another class of this embodiment, the rhodium catalyst of Step F is Rh(acac)(CH₂CH₂)₂. In another class of this embodiment, the ligand is a phosphine ligand. In a subclass of this class, the phosphine ligand is selected from: 1,2-bis(diphenyl phosphino)benzene and 1,2-bis(diphenyl phosphino)ethane. In another subclass of this class, the phosphine ligand is 1,2-bis(diphenyl phosphino)benzene. In another class of this embodiment, the solvent is selected from: tert-amyl alcohol, tert-butanol, THF, and dioxane. In a subclass of this class, the solvent is tert-amyl alcohol. In another class of this embodiment, the reaction of Step F is run at a temperature of about room temperature to about 45° C. In another class of this embodiment, the reaction of Step F, wherein the protecting group P is Boc, is run in the presence of Rh(acac)(CH₂CH₂)₂ and 1,2-bis(diphenyl phosphino)benzene. In a subclass of this class, the reaction of Step F is run in tert-amyl alcohol. In another subclass of this class, the reaction of Step F is run at a temperature of about room temperature to about 45° C. In another class of this embodiment, the process further comprises isolating the N-tert-butyl sulfinyl amine of formula 8 of Step F. In a subclass of this class, the N-tert-butyl sulfinyl amine of formula 8 is a solid. In another class of this embodiment, the process further comprises working up the reaction of Step F and using the N-tert-butyl sulfinyl amine of formula 8 in solution for Step G.

In another class of this embodiment, the protecting group P of Step G is CBZ. In another class of this embodiment, the protecting group P of Step G is Boc. In another class of this embodiment, the tert-butyl sulfoxide group of Step G is cleaved with an acid. In another class of this embodiment, the tert-butyl sulfoxide group is cleaved by treatment with an acid selected from the group consisting of: hydrochloric acid, sulfuric acid, phosphoric acid and trifluoroacetic acid. In another class of this embodiment, the cleavage of Step G is run in a halogenated solvent. In a subclass of this class, the halogenated solvent is selected from: dichloromethane, chloroform and carbon tetrachloride. In another subclass of this class, the halogenated solvent is dichloromethane. In another class of this embodiment, the cleavage of Step G is run at room temperature. In another class of this embodiment, the tert-butyl sulfoxide group of compound 8 in Step G is cleaved by treatment with hydrochloric acid. In another class of this embodiment, the process further comprises isolating the compound of formula II. In a subclass of this class, the compound of formula II is a solid. In another class of this embodiment, the process further comprises working up the reaction of Step G and using the compound of formula Ha in solution for the coupling reaction to give compound 20.

Another embodiment of the present invention provides a process for preparing a compound of formula III, or a salt thereof,

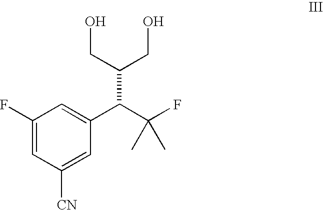

comprising the steps of:

(A) preparing a compound of formula 12:

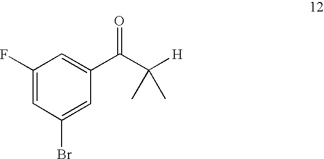

by treatment of a compound of formula 11

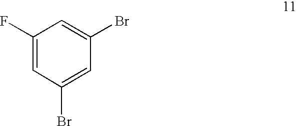

with a Grignard reagent, followed by treatment with isobutyryl chloride;

(B) forming a fluoro ketone compound of formula 13:

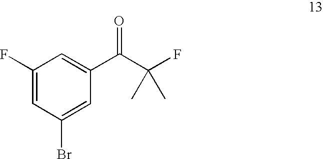

by fluorinating the compound of formula 12 by treatment with a fluorine source, and a base in the presence of a silyl halide or silyl triflate;
(C) preparing a compound of formula 14:

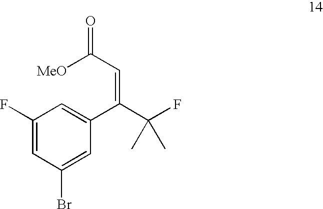

by treating the compound of formula 13 with trimethylphosphonoacetate in the presence of a base;
(D) preparing a compound of formula 15:

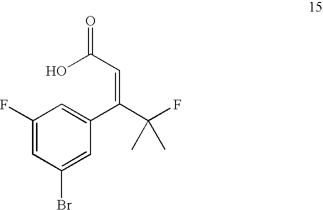

by hydrolyzing the ester of the compound of formula 14;

(E) forming a compound of formula 16:

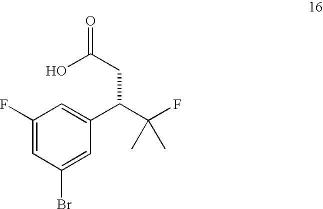

by reducing the double bond of compound of formula 15;
(F) forming a compound of formula 17:

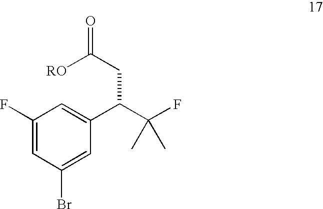

wherein R=C₁₋₃alkyl, by esterification of the compound of formula 16;

(G) forming a compound of formula 18:

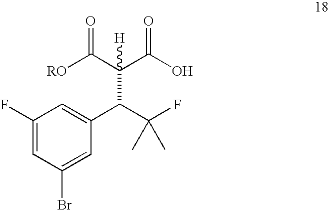

wherein R=C₁₋₃alkyl, by carboxylation of the compound of formula 17;

(H) forming a compound of formula 19:

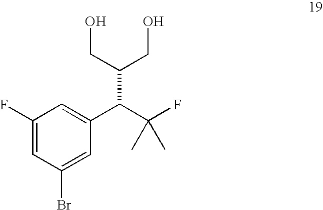

by reducing the compound of formula 18; and
(I) forming a compound of formula III

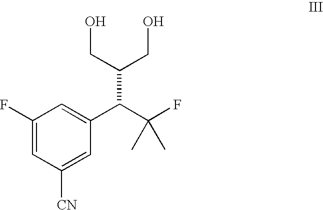

by cyanating the compound of formula 19.

In another class of this embodiment, R is —CH₃. In another class of this embodiment, R is CH₂CH₃. In yet another class of this embodiment, R is —CH₂CH₂CH₃ or —CH(CH₃)₂.

In another class of this embodiment, the Grignard reagent of Step A is isopropyl magnesium chloride. In another class of this embodiment, the reaction Step A is run in the presence of one or more transition metal halide salt catalysts. In a subclass of this class, the transition metal halide salt catalyst is selected from CuCl, ZnCl₂ and CoCl₂. In another subclass of this class, the transition metal halide salt catalysts are CuCl and ZnCl₂. In another class of this embodiment, the Grignard reaction of Step A is run in an ether solvent. In a subclass of this class, the ether solvent is tetrahydrofuran. In another class of this embodiment, the reaction of Step A is run in tetrahydrofuran with isopropyl magnesium chloride, in the presence of CuCl and ZnCl₂. In another class of this embodiment, the process further comprises isolating the compound of formula 12. In yet another class of this embodiment, the process further comprises working up the reaction of Step A and using the compound of formula 12 in a toluene solution for Step B.

In another class of this embodiment, the fluorine source of Step B is Select-Fluor™ fluorinating agent. In another class of this embodiment, the base of Step B is an alkoxide base or sodium amylate. In a subclass of this class, the alkoxide base is potassium tert-butoxide. In another class of this embodiment, the base of Step B is sodium amylate. In another class of this embodiment, the silyl halide and silyl triflate in Step B are selected from: tert-butyldimethylsilyl chloride, trimethyl silyl chloride, and tert-butyldimethylsilyl triflate. In a subclass of this class, the silyl halide of Step B is tert-butyldimethylsilyl chloride. In another class of this embodiment, the fluorination source of Step B is Select-Fluor™ fluorinating agent, the base is sodium amylate and the silyl halide is tert-butyldimethylsilyl chloride. In another class of this embodiment, the process further comprises isolating the compound of formula 13. In yet another class of this embodiment, the process further comprises working up the reaction of Step B and using the compound of formula 13 in a toluene solution for Step C.

In a class of this embodiment, the base of Step C is selected from: cesium carbonate, potassium carbonate, lithium carbonate, potassium tert-butoxide, lithium hydride, sodium hydride, and sodium amylate. In a subclass of this class, the base of Step C is potassium carbonate. In another class of this embodiment, the trimethylphosphonoacetate of Step C is pretreated with base before addition to the compound of formula 13. In a subclass of this class, the base in Step C is selected from: cesium carbonate, potassium carbonate, lithium carbonate, potassium tert-butoxide, lithium hydride, sodium hydride, and sodium amylate. In a subclass of this subclass, the base of Step C is potassium carbonate. In another class of this embodiment, the reaction of Step C is run in a polar aprotic solvent. In a subclass of this class, the polar aprotic solvent is selected from: dimethyl formamide, tetrahydrofuran or ether. In a subclass of this subclass, the solvent of Step C is dimethyl formamide. In another class of this embodiment, the compound of formula 13 in Step C is reacted with trimethylphosphonoacetate which was pretreated with potassium carbonate. In a subclass of this class, the reaction in Step C is run in dimethyl formamide. In another class of this embodiment, the process further comprises isolating the compound of formula 14 of Step C. In yet another class of this embodiment, the process further comprises working up the reaction of Step C and using compound of formula 14 in toluene for Step D.

In another class of this embodiment, the hydrolysis of Step D is run using sodium hydroxide, lithium hydroxide or potassium hydroxide. In a subclass of this class, the hydrolysis of Step D is run in using sodium hydroxide. In another subclass of this class, the hydrolysis of Step D is run in an aqueous solvent. In a subclass of this subclass, the aqueous solvent is methanol/water. In another subclass of this subclass, the aqueous solvent is methanol/water/toluene. In another class of this embodiment, the hydrolysis of Step D is run using sodium hydroxide in methanol/water. In another class of this embodiment, the hydrolysis of Step D is run using sodium hydroxide in methanol/water/toluene. In another class of this embodiment, the process further comprises isolating the compound of formula 15 of Step D.

In another class of this embodiment, the reduction of compound 15 of Step E is a hydrogenation in the presence of hydrogen and a ruthenium catalyst. In a subclass of this class, the ruthenium catalyst has an axial chiral ligand. In a subclass of this subclass, the axial chiral ligand is a Josiphos™ type ligand, a Solphos™ type ligand, a CH₃O-BIPHEP™ type ligand, a BINAP type ligand, or a Segphos™ type ligand. In another subclass of this subclass, the axial chiral ligand is (R)—Cl, CH₃O-BIPHEP™, (S)-Solphos™, (R)-Furyl-Solphos™ and SL-J212-1. In another subclass of this subclass, the Josiphos™ type axial chiral ligand is SL-J212-1. In another subclass of this class, the hydrogenation of Step E is run under pressure. In a subclass of this subclass, the hydrogenation of Step E is run at 200 psig. In another subclass of this class, the hydrogenation of Step E is run at about 40-50° C. In another class of this embodiment, the reduction of Step E is a hydrogenation in the presence of a ruthenium catalyst with a Josiphos™ type axial chiral ligand SL-J212-1. In a subclass of this class, the ruthenium catalyst is prepared by reacting [(cymene)RuCl]₂ with SL-J212-1. In another class of this embodiment, the process further comprises isolating the compound of formula 16 of Step E. In another class of this embodiment, the process further comprises isolating the compound of formula 16 of Step E as a solid and recrystallizing the compound of formula 16. In another class of this embodiment, the process further comprises working up the reaction of Step E and using the compound of formula 16 in solution for Step F. In yet another class of this embodiment, the process further comprises working up the reaction of Step E and using the compound of formula 16 in a methanolic solution for Step F.

In another class of this embodiment, the esterification of Step F is run in the presence of an acid chloride in an alcohol solvent. In a subclass of this class, the ester formed by the esterification in Step F is a methyl ester and the esterification is run in the presence of acetyl chloride in methanol. In a subclass of this subclass, the esterification is run at room temperature. In another class of this embodiment, the process further comprises isolating the compound of formula 17 of Step F.

In another class of this embodiment, the carboxylation of Step G is run by treating the compound of formula 17 with a base and in an ether or polar solvent, followed by the addition of CO₂. In a subclass of this class, the base is selected from: lithium hexamethyl disilazide, sodium hexamethyl disilazide, potassium hexamethyl disilazide and LDA. In another subclass of this class, the ether or polar solvent is selected from one or more of: THF, THF, MTBE, DME, toluene, and DMPU. In another subclass of this class, the carboxylation reaction was run by treating the compound of formula 17, in THF or toluene, with lithium hexamethyl disilazide, followed by the addition of CO₂. In another subclass of this class, the carboxylation reaction was run by treating the compound of formula 17, in THF or toluene and DMPU, with lithium hexamethyl disilazide, followed by the addition of CO₂. In another subclass of this class, the carboxylation reaction was run by treating the compound of formula 17, in DME/toluene and DMPU, with lithium hexamethyl disilazide, followed by the addition of CO₂. In another class of this embodiment, the process further comprises isolating the compound of formula 18 of Step G.

In a class of this embodiment, the reduction of Step H was run in the presence of a reducing agent. In a subclass of this class, the reducing agent is sodium borohydride, sodium borohydride/I₂, sodium borohydride/Br₂, sodium borohydrideBF₃/tetrahydrofuran complex, BF₃/etherate complex, borane/tetrahydrofuran complex, and borane/dimethyl sulfide complex. In another subclass of this class, the reducing agent is sodium borohydride/Br₂. In another subclass of this class, the reduction of Step H is run in a solvent selected from one or more of: toluene, DME, THF, DME/toluene, and dichloromethane. In another subclass of this class, the reduction of Step H is run in a solvent selected from DME and toluene. In another class of this embodiment, the process further comprises isolating the compound of formula 19 of Step H.

In a class of this embodiment, the cyanation of Step I is run in the presence of zinc, bromine, Zn(CN)₂ and a palladium catalyst. In a subclass of this class, the palladium catalyst is a bidentate or monodentate palladium catalyst. In another subclass of this class, the palladium phospine catalyst. In a subclass of this subclass, the palladium catalyst is palladium tetrakis triphenylphosphine. In another subclass of this class, the palladium catalyst is Pd(dppf)₂. In another subclass of this class, the cyanation of Step I is run in DMF. In another subclass of this class, the cyanation of Step I is run in DMF at 80° C.

In another class of this embodiment, the process further comprises isolating the compound of formula III of Step I.

Another embodiment of the present invention provides for a method of preventing or treating a disease related to CB-1 modulation comprising administering a therapeutically effective amount of a polymorph, hydrate or salt of Compound Ito a subject in need thereof.

Another embodiment of the present invention provides for the use of a therapeutically effective amount of a polymorph, solvate, hydrate or salt of Compound I for the manufacture of a medicament useful for the treatment, control, or prevention of a disease related to CB-1 modulation in a subject in need of such treatment.

Another embodiment of the present invention provides for a method of preventing or treating obesity, eating disorders, or an obesity related disorder comprising administering a therapeutically effective amount of a polymorph, hydrate or salt of Compound Ito a subject in need thereof.

Another embodiment of the present invention provides for the use of a therapeutically effective amount of a polymorph, solvate, hydrate or salt of Compound I for the manufacture of a medicament useful for the treatment, control, or prevention of obesity, eating disorders, or an obesity-related disorder in a subject in need of such treatment.

The term “3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile” comprises not only the solid form of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, but also any amorphous or partially crystalline solid form of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile, such as glasses, lyophilates, and mixtures thereof, which may be converted to 3-[(1S)-1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile through warming.

Polymorphs are compounds having the same chemical composition but different crystal structures. Polymorphism is the ability of the same chemical substance to exist as different crystalline structures. The compound 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile of structural formula I, and the HCl salt thereof, has been found it exist in at least eleven polymorphic or crystalline forms each of which can be formed by careful control of the crystallization conditions.

The term “hydrate” is meant to include all full, multiple and partial hydrates of compound I, including, but not limited to, the mono hydrate, hemi-hydrate and bis hydrate.

The term “solvate” is meant to include compound forms containing solvent molecules within the crystal structure of Compound I, or solvent molecules bound to or associated with Compound I, including but not limited to toluene, heptane, isopropyl acetate, ethyl acetate, methyl cyclohexane and water.

The term “amorphous” refers to solid forms that have no long-range molecular order.

The 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile of structural formula I has been found to form crystalline hydrochloric acid salts.

Additional salts of compounds of formula I refer to the pharmaceutically acceptable and common salts, for example, base addition salt to carboxyl group when the compound has a carboxyl group, or acid addition salt to amino or basic heterocycle when the compound has an amino or basic heterocycle group, and the like. The term “pharmaceutically acceptable salts” refers to salts prepared from pharmaceutically acceptable non-toxic bases or acids including inorganic bases or acids and organic bases or acids. The base addition salts include salts with alkali metals (including, but not limited to, sodium, potassium); alkaline earth metals (including, but not limited to, calcium, magnesium); ammonium or organic amines (including, but not limited to, trimethylamine, triethylamine, dicyclohexylamine, ethanolamine, diethanolamine, triethanolamine, procaine, N,N′-dibenzylethylenediamine), and the like. The acid addition salts include salts with inorganic acids (including, but not limited to, hydrochloric acid, sulfuric acid, nitric acid, phosphoric acid, perchloric acid), organic acids (including, but not limited to, acetic acid, oxalic acid, maleic acid, fumaric acid, tartaric acid, citric acid, ascorbic acid, trifluoroacetic acid, acetic acid), sulfonic acids (including, but not limited to, methanesulfonic acid, isethionic acid, benzenesulfonic acid, p-toluenesulfonic acid, p-toluenesulfonic acid monohydrate, p-toluene sulfonic acid hydrate, camphor sulfonic acid), and the like.

In one embodiment of the present invention there is provided a pharmaceutical composition comprising 3-[(1S)-1-(1-{(S)-(4-chlorophenyl)[3-(5-oxo-4,5-dihydro-1,3,4-oxadiazol-2-yl)phenyl]-methyl}azetidin-3-yl)-2-fluoro-2-methylpropyl]-5-fluorobenzonitrile (Compound I) as a free base, salt, hydrate or polymorph thereof. In a class of this embodiment, Compound I is in substantially pure form. In another class of this embodiment, Compound I is crystalline. In another class of this embodiment, Compound I is crystalline anhydrous free base. In another class of this embodiment, Compound I is a crystalline free base solvate. In another class of this embodiment, Compound I is a crystalline anhydrous salt. In another class of this embodiment, Compound I is a crystalline salt hydrate. In another class of this embodiment, Compound I is a crystalline anhydrous HCl salt. In another class of this embodiment, Compound I is a crystalline HCl salt hydrate. In another class of this embodiment, the composition comprises 3-[(1S)-1-(1-{(S)-(4-chlorophenyl) [3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I) as the free base Form I of Compound I. In a subclass of this class, the free base Form I of Compound I is in substantially pure form. In another subclass of this class, free base Form I of Compound I is crystalline. In another subclass of this class, free base Form I of Compound I is anhydrous. In another subclass of this class, free base Form I of Compound I is anhydrous and crystalline.

In another class of this embodiment, the composition comprises 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I) as the free base toluene/heptane solvate Form I, Type B of Compound I. In a subclass of this class, the free base toluene/heptane solvate Form I, Type B of Compound I is in substantially pure form. In another subclass of this class, the free base toluene/heptane solvate Form I, Type B of Compound I is crystalline. In another subclass of this class, the free base toluene/heptane solvate Form I, Type B of Compound I is anhydrous. In another subclass of this class, the free base toluene/heptane solvate Form I, Type B of Compound I is anhydrous and crystalline.

In another class of this embodiment, the composition comprises 3-[(1S)-1-(1-{(S)-(4-chloro-phenyl)[3-(5-oxo-4,5-dihydro-1,3,4-oxadiazol-2-yl)phenyl]methyl}azetidin-3-yl)-2-fluoro-2-methyl-propyl]-5-fluorobenzonitrile (Compound I) as the free base isopropyl acetate/methyl cyclohexane solvate Form I, Type A of Compound I. In a subclass of this class, the free base isopropyl acetate/methyl cyclohexane solvate Form I, Type A of Compound I is in substantially pure form. In another subclass of this class, the free base isopropyl acetate/methyl cyclohexane solvate Form I, Type A of Compound I is crystalline. In another subclass of this class, the free base isopropyl acetate/methyl cyclohexane solvate Form I, Type A of Compound I is anhydrous. In another subclass of this class, the free base isopropyl acetate/methyl cyclohexane solvate Form I, Type A of Compound I is anhydrous and crystalline.

In another class of this embodiment, the composition comprises 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I) as the HCl salt Form A of Compound I. In a subclass of this class, the HCl salt Form A of Compound I is in substantially pure form. In another subclass of this class, the HCl salt Form A of Compound I is crystalline. In another subclass of this class, the HCl salt Form A of Compound I is anhydrous. In another subclass of this class, the HCl salt Form A of Compound I is anhydrous and crystalline.

In another class of this embodiment, the composition comprises 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I) as the HCl salt Form B of Compound I. In a subclass of this class, the HCl salt Form B of Compound I is in substantially pure form. In another subclass of this class, the HCl salt Form B of Compound I is crystalline. In another subclass of this class, the HCl salt Form B of Compound I is anhydrous. In another subclass of this class, the HCl salt Form B of Compound I is anhydrous and crystalline.

In another class of this embodiment, the composition comprises 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I) as the HCl salt Form C of Compound I. In a subclass of this class, the HCl salt Form C of Compound I is in substantially pure form. In another subclass of this class, the HCl salt Form C of Compound I is crystalline. In another subclass of this class, the HCl salt Form C of Compound I is anhydrous. In another subclass of this class, the HCl salt Form C of Compound I is anhydrous and crystalline.

In another class of this embodiment, the composition comprises 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I) as the HCl salt Form D of Compound I. In a subclass of this class, the HCl salt Form D of Compound I is in substantially pure form. In another subclass of this class, the HCl salt Form D of Compound I is crystalline. In another subclass of this class, the HCl salt Form D of Compound I is anhydrous. In another subclass of this class, the HCl salt Form D of Compound I is anhydrous and crystalline.

In another class of this embodiment, the composition comprises 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I) as the HCl salt Form E of Compound I. In a subclass of this class, the HCl salt Form E of Compound I is in substantially pure form.

In another subclass of this class, the HCl salt Form E of Compound I is crystalline. In another subclass of this class, the HCl salt Form E of Compound I is anhydrous. In another subclass of this class, the HCl salt Form E of Compound I is anhydrous and crystalline.

In another class of this embodiment, the composition comprises 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I) as the HCl salt Form F of Compound I. In a subclass of this class, the HCl salt Form F of Compound I is in substantially pure form. In another subclass of this class, the HCl salt Form F of Compound I is crystalline. In another subclass of this class, the HCl salt Form F of Compound I is a hydrate. In another subclass of this class, the HCl salt Form F of Compound I is a crystalline hydrate.

In another class of this embodiment, the composition comprises 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I) as the HCl salt Form G of Compound I. In a subclass of this class, the HCl salt Form G of Compound I is in substantially pure form. In another subclass of this class, the HCl salt Form G of Compound I is crystalline. In another subclass of this class, the HCl salt Form G of Compound I is anhydrous. In another subclass of this class, the HCl salt Form G of Compound I is anhydrous and crystalline.

In another class of this embodiment, the composition comprises 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I) as the HCl salt Form H of Compound I. In a subclass of this class, the HCl salt Form H of Compound I is in substantially pure form. In another subclass of this class, the HCl salt Form H of Compound I is crystalline. In another subclass of this class, the HCl salt Form H of Compound I is anhydrous. In another subclass of this class, the HCl salt Form H of Compound I is anhydrous and crystalline.

The compounds in the processes of the present invention include stereoisomers, such as optical isomers, diastereomers and geometerical isomers, or tautomers depending on the mode of substitution. The present invention is meant to comprehend all such isomeric forms of the compounds in the compositions of the present invention, and their mixtures. All hydrates, solvates and polymorphic crystalline forms of the above-described compounds and their use, including their use in the processes of the instant invention, are encompassed within scope of the instant invention.

Neurokinin-1 (NK-1) receptor antagonists may be favorably employed in combination with a compound of the present invention. NK-1 receptor antagonists of use in the present invention are fully described in the art. Specific neurokinin-1 receptor antagonists of use in the present invention include: (±)-(2R3R,2S3S)—N-{[2-cyclopropoxy-5-(trifluoromethoxy)-phenyl]methyl}-2-phenylpiperidin-3-amine; 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine; aperpitant; CJ17493; GW597599; GW679769; R673; R067319; R1124; R1204; SSR146977; SSR240600; T-2328; and T2763; or a pharmaceutically acceptable salts thereof. Examples of other anti-obesity agents that can be employed in combination with a compound of formula I, II or III are disclosed in “Patent focus on new anti-obesity agents,” Exp. Opin. Ther. Patents, 10: 819-831 (2000); “Novel anti-obesity drugs,” Exp. Opin. Invest. Drugs, 9: 1317-1326 (2000); and “Recent advances in feeding suppressing agents: potential therapeutic strategy for the treatment of obesity, Exp. Opin. Ther. Patents, 11: 1677-1692 (2001). The role of neuropeptide Y in obesity is discussed in Exp. Opin. Invest. Drugs, 9: 1327-1346 (2000). Cannabinoid receptor ligands are discussed in Exp. Opin. Invest. Drugs, 9: 1553-1571 (2000).

Another aspect of the present invention provides pharmaceutical compositions which comprise a polymorph, hydrate or salt of Compound I and a pharmaceutically acceptable carrier. The pharmaceutical compositions of the present invention comprise a compound of Formula I as an active ingredient or a pharmaceutically acceptable salt thereof, and may also contain a pharmaceutically acceptable carrier and optionally other therapeutic ingredients.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the polymorphs, hydrates and salts of Compound I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

The polymorphs, hydrates and salts of Compound I may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

The present invention provides a method for the treatment and/or prevention of obesity and obesity-related disorders in a subject in need thereof comprising administering a therapeutically effective amount of a hydrate, salt or polymorph of Compound Ito the subject in need thereof. The present invention also provides for the use of the hydrates, salts and polymorphs of Compound I for the manufacture of a medicament for the prevention and/or treatment of CB-1 modulated disorders, such as psychosis, memory deficits, cognitive disorders, Alzheimer's disease, migraine, neuropathy, neuro-inflammatory disorders including multiple sclerosis and Guillain-Barre syndrome and the inflammatory sequelae of viral encephalitis, cerebral vascular accidents, and head trauma, anxiety disorders, stress, epilepsy, Parkinson's disease, movement disorders, and schizophrenia. The compounds are also useful for the treatment of substance abuse disorders, the treatment of obesity or eating disorders, obesity-related disorders and complications associated therewith, including left ventricular hypertrophy, as well as the treatment of asthma, constipation, chronic intestinal pseudo-obstruction, and cirrhosis of the liver.

The obesity-related disorders herein are associated with, caused by, or result from obesity. Examples of obesity-related disorders include restenosis, atherosclerosis, arteriosclerosis, overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia, metabolic syndrome, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-esophageal reflux, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, hyperuricaemia, lower back pain, gallbladder disease, gout, kidney cancer, increased anesthetic risk, left ventricular hypertrophy, Alzheimer's disease.

“Treatment” (of obesity and obesity-related disorders) refers to the administration of the compounds or combinations of the present invention to reduce or maintain the body weight of an obese subject. “Prevention” (of obesity and obesity-related disorders) refers to the administration of the compounds or combinations of the present invention to reduce or maintain the body weight of a subject at risk of obesity.

The term “subject”, as used herein refers to an animal, preferably a mammal, most preferably a human, who has been the object of treatment, observation or experiment. The term “subject in need thereof” refers to a subject who is in need of treatment or prophylaxis as determined by a researcher, veterinarian, medical doctor or other clinician. In one embodiment, the subject in need of treatment is an obese mammal. In another embodiment, the subject in need of treatment is an obese human with one or more obesity-related co-morbidities. In another embodiment, the subject in need of treatment is an obese human without obesity-related co-morbidities. The term “therapeutically effective amount” as used herein means the amount of the active compounds in the composition that will elicit the biological or medical response in a tissue, system, subject, or human that is being sought by the researcher, veterinarian, medical doctor or other clinician, which includes alleviation of the symptoms of the disorder being treated.

The magnitude of prophylactic or therapeutic dose of the salt, hydrate or polymorph of compound I will, of course, vary with the nature of the severity of the condition to be treated and with the particular compound in the composition and its route of administration. It will also vary according to the age, weight and response of the individual patient. In general, for treating obesity or an obesity-related disorder, the daily dose range of a salt, hydrate or polymorph of compound I is administered at a daily dosage of from about 0.0001 mg/kg to about 100 mg/kg, preferably from about 0.001 mg/kg to about 100 mg/kg, more preferably from about 0.001 mg/kg to about 10 mg/kg of body weight of a subject in single or divided doses two to six times a day, or in sustained release form. On the other hand, it may be necessary to use dosages outside these limits in some cases. The compounds of this invention can be administered to humans in the dosage ranges specific for each compound. For oral administration, the compositions are preferably provided in the form of tablets containing from 0.01 mg to 1,000 mg, preferably 0.01, 0.05, 0.1, 0.2, 0.5, 1.0, 1.5, 2.0, 2.5, 3.0, 3.5, 4.0, 4.5, 5.0, 5.5, 6.0, 7.5, 10, 15, 20, 25, 30, 40, 50, 60, 75, 80, 100, 125, 150, 175, 200, 225, 250, 500, 750, 850 and 1,000 milligrams of each active ingredient for the symptomatic adjustment of the dosage to the subject to be treated. This dosage regimen may be adjusted to provide the optimal therapeutic response.

The X-ray powder diffraction pattern of the crystalline forms of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I), and the salts, hydrates and solvates thereof, were generated on a Philips Analytical X'Pert PRO X-ray Diffraction System with PW3040/60 console. A PW3373/00 ceramic Cu LFF X-ray tube K-Alpha radiation was used as the source. The X-ray powder diffraction spectrum was recorded at ambient temperature.

DSC data were acquired using a TA Instruments DSC-2910 differential scanning calorimeter at a heating rate of 10° C./min under N₂ flow. TA Instruments DSC 2910 or equivalent instrumentation. Between 2 and 6 mg sample is weighed into an open aluminum pan. This pan is then crimped and placed at the sample position in the calorimeter cell. The sample is heated in a closed pan. An empty pan is placed at the reference position. The calorimeter cell is closed and a flow of nitrogen is passed through the cell. The heating program is set to heat the sample at a heating rate of 10° C./min to a temperature of approximately 250° C. The heating program is started. When the run is completed, the data are analyzed using the DSC analysis program contained in the system software. The melting endotherm is integrated between baseline temperature points that are above and below the temperature range over which the endotherm is observed. The data reported are the onset temperature, peak temperature and enthalpy.

TGA data were acquired using Perkin Elmer TGA-7 thermogravimetric analyzer. Between 5 and 20 mg sample is weighed into a platinum pan. The furnace is raised and a flow of nitrogen is passed over the sample. The heating program is set to heat the sample under a nitrogen flow at a heating rate of 10° C./min to a temperature of approximately 250° C. The heating program is started. When the run is completed, the data are analyzed using the delta Y function in the analysis program contained in the system software. The percent weight loss by the sample is calculated from the onset of the heating program to the melt/decomposition of the sample.

In the schemes and examples below, various reagent symbols and abbreviations have the following meanings: acac is acetyl acetonate; aq is aqueous; t-AmOH is tert-amyl alcohol; BINAP is 2,2′-bis(diphenylphosphino)-1,1′-binaphthyl; BuLi or n-BuLi is butyl lithium; Bu₂Mg is dibutyl magnesium; Boc is tert-butoxy carbonyl; Boc anhydride is tert-butoxy carbonyl anhydride; CBZ is carbobenzyloxy; CDI is 1,1′-carbonyldiimidazole; DABCO is 1,4-diazabicyclo[2.2.2]-octane; DME is ethylene glycol dimethyl ether; DMF is dimethylformamide; DMPU is 1,3-dimethyl-3,4,5,6-tetrahydro-2(1H)-pyrimidinone; dppbenz is 1,2-bis(diphenyl phosphino)benzene; dppe is diphenyl phosphino ethene; dppf=(phenyl)₂PC₅H₄FeC₅H₄P(phenyl)₂, g is gram; h is hour(s); GMP is good manufacturing practices; HCl is hydrochloric acid; IPA is isopropyl alcohol; IPAc or iPAc is isopropyl acetate; i-ProH is isopropanol; KF is Karl Fischer; kg is kilogram; L is liter; LCAP is liquid chromatography analytical purity; LDA is lithium diisopropylamide; LiHMDS is lithium hexamethyl disilazide; Me is methyl; MeOH is methanol; min is minute(s); mL is milliliter; mol is mole; mmol is millimole; MTBE is tert-butyl methyl ether; N is normal; PPTS is pyridinium p-toluene sulfonate; rt is room temperature; SL-J212-1 is (R)-1-[(S)-2-Di-2-furyl-phosphino)-ferrocenyl]ethyldi-tert.-butyl-phosphine, TBSC1 is tert-butyl dimethyl silyl chloride; TFA is trifluoroacetic acid, and THF is tetrahydrofuran.

A representative experimental procedure utilizing the novel process is detailed in the schemes and examples below. The following Schemes and Examples are provided to illustrate the invention and are not to be construed as limiting the scope of the invention in any manner.

The invention describes an efficient process for the synthesis of the potent CB-1 inverse agonist Compound I. As shown in Scheme 1, the fully functionalized chiral benzhydrylamine II is synthesized from the commercially available 3-bromobenzoyl chloride 1. 3-Bromobenzoyl chloride 1 was converted to hydrazide 3 by treatment with DABCO and hydrazine. Hydrazide 3 was treated with CDI to give oxadiazole 4, which was converted to aldehyde 5 via a metal-halogen exchange reaction. Aldehyde 5 was converted to the Ellman's Imine compound 6 by treatment with PPTS and (S)-sulfinamide. The oxadiazole nitrogen of compound 6 was then protected with a Boc group to give N-Boc Imine 7, followed by a highly stereoselective Rh-catalyzed addition of arylboroxine 10 to N-Boc Imine 7 to provide sulfinamide 8. The selective deprotection of sulfinamide 8 in the presence of the protected oxadiazole provided benzhydrylamine II.

Scheme 2 illustrates the synthesis of the cyanodiol III intermediate. The synthesis of cyanodiol III was commenced from 1,3-dibromofluorobenzene 11 via Grignard formation with isopropylmagnesium chloride followed by CuCl/ZnCl₂ catalyzed addition to isobutyryl chloride which afforded ketone 12. Formation of the silyl enol ether of ketone 12 followed by in situ fluorination with Select-Fluor™ fluorinating agent provided fluoro ketone 13. Fluoro ketone 13 was treated with potassium carbonate and trimethylphosphonoacetate to give the Horner-Wadsworth-Emmons adduct, α,β-unsaturated ester 14; this compound was then hydrolyzed in situ with NaOH to give the α,β-unsaturated acid 15. Rhodium catalyzed asymmetric hydrogenation of α,β-unsaturated acid 15 gave saturated acid 16, which, following in situ esterification, provided saturated ester 17. Carboxylation of saturated ester 17 afforded β-carboxyester 18, which was reduced to bromodiol 19 via reduction with sodium borohydride-bromine in DME. Finally, the palladium catalyzed cyanation of bromo diol 19 yielded the requisite cyanodiol III.

Scheme 3 illustrates a highly convergent coupling of benzhydrylamine II with the cyanodiol III used to install the azetidine ring of intermediate 20. Finally, the synthesis of Compound I is completed via removal of the N-Boc protecting group from intermediate 20. All of the intermediates in this route are crystalline, which is an advantage of this invention with regard to isolation and purification.

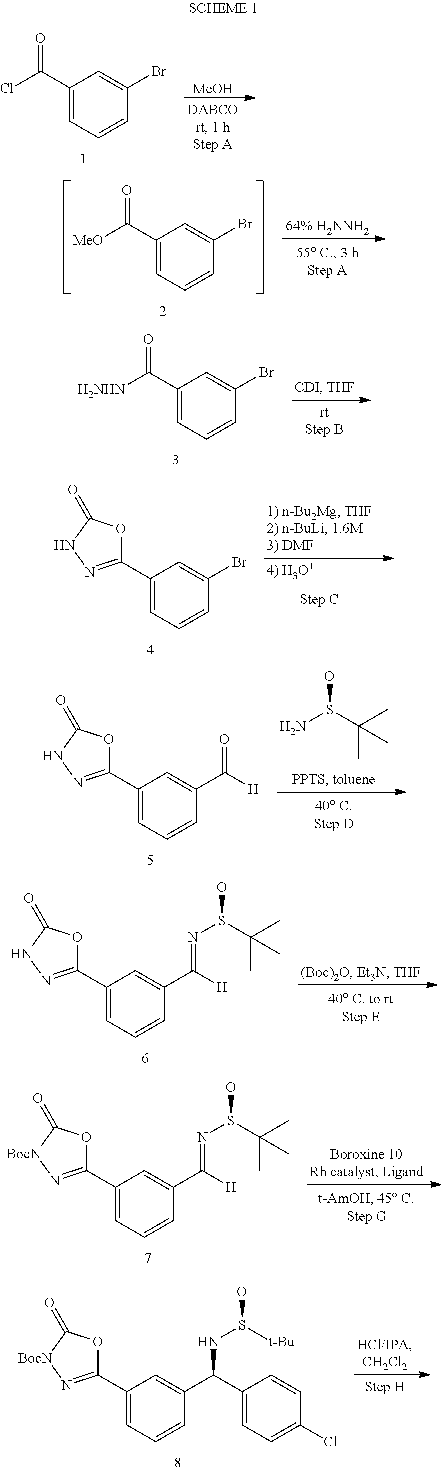

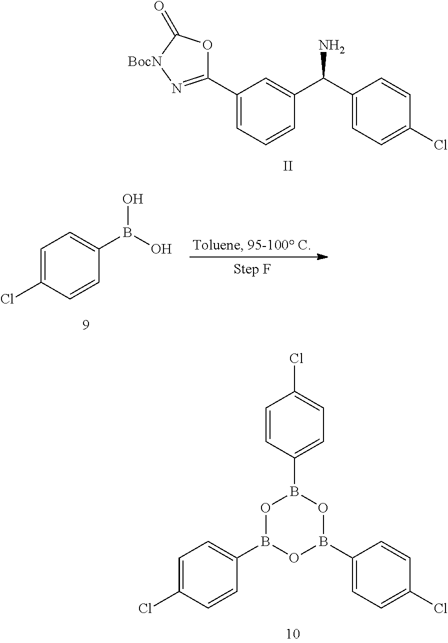

### Example 1

### Preparation of tert-butyl-5-{3-[(S)-amino(4-chlorophenyl)methyl]phenyl}-2-oxo-1,3,4-oxadiazole-3(2H)-carboxylate (Compound II)

Step A: Preparation of Hydrazide 3. To a 100 L round bottom flask under nitrogen was added DABCO (2.81 kg, 25.06 mol) and MeOH (35 L). 3-bromobenzoyl chloride 1 (5.0 kg, 22.78 mol) was charged over 30 min at 20-25° C. and an ice water bath was used to control the temperature. The mixture was stirred at room temperature for 10-20 minutes. Hydrazine (64%, 8.8 L, 182 mol) was added over 20 minutes, and the reaction mixture was heated at 50 to 55° C. for 3 hours. Water (35 L) was added to crystallize the batch at room temperature over 1 hour. The resulting slurry was stirred at room temperature for 1-2 hours and filtered. The wet cake was washed with water (3×15 L), and dried at room temperature under a vacuum/N₂ sweep to afford hydrazide 3 as white solid. HPLC retention time of hydrazide 3=6.75 minutes, on Waters Symmetry C-18 column, 5 micron, 4.6×250 mm; 20° C., UV detection at 215 nm; gradient flow 1.0 mL/min; A=water with 0.1% H₃PO₄; B=acetonitrile; gradient elution: 0 minutes: 95% A/5% B; 5 minutes: 55% A/45% B; 10 minutes: 25% A/75% B; 13 minutes: 10% A/90% B; and 26 minutes: 10% A/90% B. 1H NMR (DMSO-d₆): [symbol] 9.88 (1H, s), 7.98 (1H, m), 7.81 (1H, m), 7.70 (1H, m), 7.41 (1H, m), 4.52 (2H, s).

Step B: Preparation of Oxadiazole 4. To a 75 L round bottom flask under nitrogen was added 3-bromobenzoic hydrazide 3 (3.5 kg, 16.3 mol) and THF (35 L). The slurry was stirred at room temperature for 5-10 min. Then CDI (3.17 kg, 19.5 mol) was added over 10-20 min at 20-25° C., and an ice water bath was used to control the temperature. The reaction mixture turned to a clear solution gradually, and the solution was stirred at room temperature for 2-3 h. IPAc (35 L) and water (35 L) were added to the solution. The layers were separated and the aqueous layer was extracted with IPAc (15 L). The combined organic layers were washed with water (2×35 L), followed by brine (20 L), and concentrated. A slurry formed during concentration. The slurry was flushed with heptane (2×10 L), and the final volume was adjusted to 30 L. The resulting slurry mixture was stirred at room temperature for 1-2 hours, then filtered. The resulting wet cake was washed with heptane (2×10 L), and dried at room temperature under a vacuum/N₂ sweep to afford oxadiazole 4 as white solid. HPLC retention time of oxadiazole 4=11.20 minutes on Waters Symmetry C-18 column, 5 micron, 4.6×250 mm; 20° C., UV detection at 215 nm; gradient flow 1.0 mL/min; A=water with 0.1% H₃PO₄; B=acetonitrile; gradient elution: 0 minutes: 95% A/5% B; 5 minutes: 55% A/45% B; 10 minutes: 25% A/75% B; 13 minutes: 10% A/90% B; and 26 minutes: 10% A/90% B. ¹H NMR (400 MHz, dmso-d₆) [symbol] 12.65 (1H, s), 7.85 (1H, s), 7.72 (2H, m), 7.42 (1H, t, J=7.8 Hz).

Step C: Preparation of Aldehyde 5. To a 75 L round bottom flask under nitrogen was added oxadiazole 4 (3.3 kg, 13.7 mol) and THF (33 L). The solution was cooled to −50° C., and Bu₂Mg (1M in heptane, 2N, 10.3 L, 10.3 mol) was added over 30-50 min at −40 to −45° C. A dry ice acetone bath was used to control the temperature. The resulting heterogeneous mixture was stirred at the same temperature for 1 hour. Then n-BuLi (1.6M in hexane, 10.3 L, 16.6 mol) was then added over 30 min at −40 to −45° C. A dry ice acetone bath was used to control the temperature. The slurry was stirred at −40 to −45° C. for 2-3 hours, then DMF (3.2 L, 41.1 mol) was added over 1 hour at −40° C. The reaction mixture was allowed to warm to 0 to 10° C. and stirred at the same temperature for 3-5 h. The batch was cooled to 0° C. and the reaction was quenched by adding 2N HCl (10 L) over 20 minutes keeping the batch temperature below 10° C. The batch was transferred to 160 L extractor, and EtOAc (33 L) and 2N HCl (23 L) were added. The resulting layers were separated and the aqueous layer was extracted with EtOAc (10 L). The combined organic layers was washed with water (33 L) and followed by brine (20 L), concentrated, and flushed with heptane (10 L). The slurry was stirred in 1:2 EtOAc and heptane (12 L) at room temperature for 2 hours, and filtered. The wet cake was washed with heptane (2×8 L), and dried at room temperature under a vacuum/N₂ sweep to afford aldehyde 5 as a white solid. HPLC retention time of Aldehyde 5=8.73 minutes on Waters Symmetry C-18 column, 5 micron, 4.6×250 mm; 20° C., UV detection at 215 nm; gradient flow 1.0 mL/min; A=water with 0.1% H₃PO₄; B=acetonitrile; gradient elution: 0 minutes: 95% A/5% B; 5 minutes: 55% A/45% B; 10 minutes: 25% A/75% B; 13 minutes: 10% A/90% B; and 26 minutes: 10% A/90% B. ¹H NMR (400 MHz, dmso-d₆) δ 12.65 (1H, s), 10.04 (1H, s), 8.28 (1H, s), 8.11 (2H, m), 7.79 (1H, m).

Step D: Preparation of N-tert-butane Sulfinyl Imine 6. To a 75 L round bottom flask under nitrogen was added aldehyde 5 (2.1 kg, 11.0 mol), (S)-tert-butane sulfinamide (1.46 kg, 12.1 mol), PPTS (1.38 kg, 5.5 mol) and toluene (20 L). The resulting slurry was heated to 40° C. for 20 hours. The batch was cooled to 25° C. and the resulting solid was filtered. The wet cake was washed with toluene (3×15 L), water (3×15 L) and followed by heptane (3×12 L). After the wet cake was dried at room temperature under a vacuum/N₂ sweep, Ellman's imine 6 was isolated as white solid. HPLC retention time Ellman's imine 6=10.64 minutes on Waters Symmetry C-18 column, 5 micron, 4.6×250 mm; 20° C., UV detection at 215 nm; gradient flow 1.0 mL/min; A=water with 0.1% H₃PO₄; B=acetonitrile; gradient elution: 0 minutes: 95% A/5% B; 5 minutes: 55% A/45% B; 10 minutes: 25% A/75% B; 13 minutes: 10% A/90% B; and 26 minutes: 10% A/90% B. ¹H NMR (400 MHz, dmso-d₆) δ 12.63 (1H, br s), 8.62 (1H, s), 8.27 (1H, s), 8.09 (1H, d, J=7.8 Hz), 7.95 (1H, d, J=7.8 Hz), 7.69 (1H, t, J=7.8 Hz), 1.20 (9H, s).

Step E: Preparation of N-Boc Imine 7. To a 75 L round bottom flask under nitrogen was added N-tent-butane sulfinyl imine 6 (3.1 kg, 9.83 mol), THF (39 L), Et₃N (2.5 kg, 24.7 mol) and followed by Boc anhydride (3.2 kg, 14.66 mol). The resulting mixture was heated at 40° C. for 5 h and room temperature for 8-10 hours. The mixture was concentrated and flushed with IPAc (25 L), followed by heptane (10 L). During concentration, a solid formed and the mixture volume was then adjusted to about 22 L. The slurry was stirred at room temperature for 1-2 hours and filtered. The resulting wet cake was washed with 1:4 IPAc/heptane (15 L), 1:10 IPAc/heptane (15 L) and heptane (15 L). After the wet cake was dried at room temperature under a vacuum/N₂ sweep, the N-Boc Imine product 7 was isolated as white solid. HPLC retention time of N-Boc Imine 7=14.00 minutes on Waters Symmetry C-18 column, 5 micron, 4.6×250 mm; 20° C., UV detection at 215 nm; gradient flow 1.0 mL/min; A=water with 0.1% H₃PO₄; B=acetonitrile; gradient elution: 0 minutes: 95% A/5% B; 5 minutes: 55% A/45% B; 10 minutes: 25% A/75% B; 13 minutes: 10% A/90% B; and 26 minutes: 10% A/90% B. ¹H NMR (400 MHz, CDCl₃) δ 8.64 (1H, s), 8.43 (1H, s), 8.09 (1H, d, J=7.8 Hz), 8.02 (1H, d, J=7.8 Hz), 7.63 (1H, t, J=7.8 Hz), 1.69 (9H, s), 1.30 (9H, s).

Step F: Preparation of boroxine 10. To a 50 L round bottom flask with overhead stirrer, distillation unit (batch concentrator) under nitrogen was added 4-chlorophenylboronic acid 9 (3.6 kg, 23 mol) and toluene (30 L). The resulting slurry was heated to boil (95-110° C.) and water was removed by azeotropic distillation. Fresh toluene (total about 30-35 L) was added during the distillation to maintain constant volume in the vessel. After about 30 L of solvent had distilled, the batch was cooled to room temperature and stirred for 0.5-1 hour. The product was filtered, washed with toluene (2×10 L) and followed by heptane (1×10 L), and dried at room temperature under a vacuum/N₂ sweep to afford boroxine 10 as white solid.

Step G: Preparation of Sulfinamide 8. To a 75 L round bottom flask under nitrogen was added N-Boc Imine 7 (3.1 kg, 7.88 mol), boroxine 10 (1.64 kg, 3.94 mol) and t-AmOH (62 L). The reaction mixture was heated to 45° C., and 1,2-bis(diphenyl-phosphino)benzene (93 g, 0.208 mol) was added. After the resulting mixture was sparged with nitrogen gas at 45° C. for 20 minutes, Rh(acac)(CH₂CH₂)₂ (45 g, 0.174 mol) was added. The reaction mixture was sparged with nitrogen gas at the same temperature for an additional 10 minutes, and then heated at 45° C. for 3-5 hours. The batch was cooled to room temperature and diluted with EtOAc (25 L). The resulting thin slurry was transferred to a 160 L extractor containing EtOAc (20 L) and 0.5 M aq Na₂CO₃ (22 L). After vigorous mixing for 10 minutes at room temperature, the layers were separated. The top organic layer was washed with 3% brine (3×20 L) and then brine (20 L). The organic solution was concentrated to 25-30 L, and flushed with heptane (20 L). During concentration, the product precipitated and then the batch volume was adjusted to about 60 L with heptane. The resulting slurry was stirred at room temperature for 1-2 hours, and then filtered. The resulting wet cake was washed with 1:4 EtOAc/heptane (2×20 L), and heptane (30 L). After the wet cake was dried at room temperature under a vacuum/N₂ sweep, sulfinamide 8 was isolated as pale yellow fluffy solid. HPLC retention time of sulfinamide 8=15.15 minutes on Phenomenex Synergi, 4 micron, Hydro-RP 80A, 250×4.6 mm; 20° C., UV detection at 215 nm; gradient flow 1.0 mL/min; A=water with 0.1% H₃PO₄; B=acetonitrile; gradient elution: 0 minutes: 95% A/5% B; 5 minutes: 55% A/45% B; 10 minutes: 25% A/75% B; 13 minutes: 10% A/90% B; and 26 minutes: 10% A/90% B. ¹H NMR (400 MHz, CDCl₃) δ 7.83 (1H, s), 7.74 (1H, d, J=7.7 Hz), 7.55 (1H, d, J=7.7 Hz), 7.39 (1H, t, J=7.7 Hz), 7.26 (4H, m), 5.59 (1H, s), 4.04 (1H, s), 1.60 (9H, s), 1.21 (9H, s).

Step H: Preparation of Benzhydrylamine II. To a 100-L vessel equipped with thermocouple, nitrogen flow, and overhead stirrer was charged sulfinamide 8 (3.0 kg, 5.93 mol) and dichloromethane (15 L). To the solution was charged a solution of HCl in i-PrOH (10 L of 0.62 M and 6.8 L of 0.65 M, 10.08 mol). The reaction was complete after a 3 hour age at room temperature. The reaction mixture was quenched with water (30 L, exotherm), followed by saturated aqueous NaHCO₃ (15 L) slowly to control the CO₂ evolution. The mixture was stirred at room temperature for 30 minutes. The reaction mixture was then transferred to 100-L extractor, rinsing with dichloromethane (6 L). The layers were separated. The organic layer was filtered into a 75 L vessel through a 1 micron Whatman in-line filter. The solution was concentrated and distilled azeotropically, with additional i-PrOH (6 L), at an internal temperature of ˜45° C. to give a thick slurry, and then solvent switched to dichloromethane by adding 18 L of dichloromethane and concentrating to ˜8 L. The mixture was then heated to 35° C. to completely dissolve all the solids, and i-PrOH (24 L) as an anti-solvent was slowly charged to the batch via addition funnel. After the addition of i-PrOH, the batch was slowly cooled to ambient temperature overnight. The temperature was then lowered to 0-5° C. for 2 hours. The solid was then filtered and the resulting cake slurry washed with i-PrOH (2×7.5 L) and n-heptane (3×9 L). The cake was dried in the filter pot over the weekend under nitrogen sweep and vacuum pull from the bottom to give the benzhydrylamine II. HPLC retention time of benzyhydrylamine II=8.95 on Zorbax RX C8, 5.0 micron, 4.6 mm×250 mm, P.N.: 880967; temperature: 25° C., UV detection at 210 nm, column flow is 1.0 ml/min, solvent A is acetonitrile, solvent B is H₂O buffered with 0.1% H₃PO₄; gradient elution: 0 minutes: 10% A/90% B; 1 minutes: 90% A/10% B; and 15 minutes: 98% λ/2% B. ¹H (CDCl₃) δ 7.99 (t, J=1.6 Hz, 1H), 7.80 (dt, J=7.6, 1.6 Hz, 1H), 7.57 (dt, J=7.6, 1.6 Hz, 1H), 7.44 (t, J=7.6 Hz, 1H), 7.33 (m, 2H), 7.27 (m, 2H), 5.26 (s, 1H), 1.77 (brs, 2H), 1.66 (s, 914). ¹H NMR (400 MHz, CDCl₃) δ 8.00 (1H, s), 7.80 (1H, d, J=7.7 Hz), 7.08 (1H, d, J=7.7 Hz), 7.44 (1H, t, J=7.7 Hz), 7.30 (4H, m), 5.26 (1H, br s), 1.60 (21-1, br s), 1.68 (9H, s).

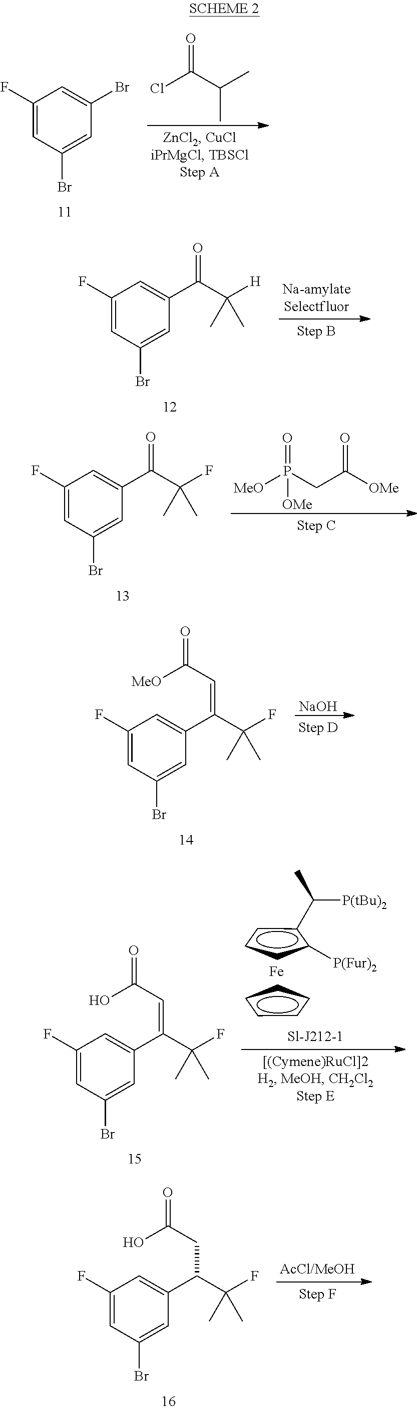

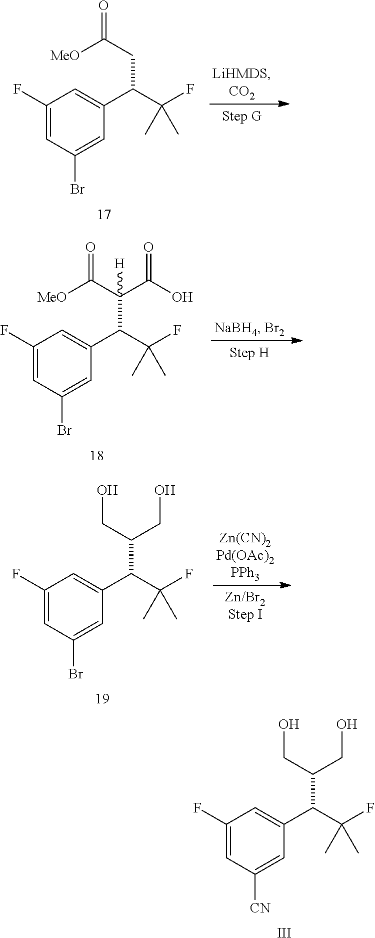

### Example 2

### Preparation of 3-Fluoro-5-{(1S)-2-fluoro-1-[2-hydroxy-1-(hydroxymethyl)ethyl]-2-methyl-propyl}benzonitrile (Compound III)

Step A: Synthesis of isopropyl ketone 12. To a 50 L round bottomed flask fitted with a thermocouple and 5 L addition funnel, under a nitrogen atmosphere, was added ZnCl₂ (32.7 g, 0.24 mol) and CuCl (23.3 g, 0.24 mol). THF (12 L) was added and the solution degassed by bubbling N₂ through the slurry for 5 minutes. Following this 2,3-dibromofluorobenzene 11 (6.0 kg, 24 mol) was added in one portion. The reaction was cooled to 10° C. and isopropyl magnesium chloride (2 M, 14 L, 28 mol) was added dropwise over 1 hour 20 minutes while maintaining the temperature between 15-20° C. A 100 L cylindrical vessel was charged with isobutyryl chloride (3.3 kg, 31 mol) and THF (6 L). The mixture was cooled to 6° C. before pumping previously prepared aryl Grignard solution into it over a 70 minute time period while maintaining the temperature between 10° C.-18° C. After complete addition, the reaction was aged for 20 minutes before being assayed for conversion. Upon complete conversion, the reaction was diluted with toluene (11 L) and 1N citric acid (10 L). The bi-phasic mixture was stirred for 10 minutes before separating the layers. The organic layer was then treated with 1N citric acid (10 L) and stirred for an additional 10 minutes. The layers were separated and the organic layer was washed sequentially with 1N K₂HPO₄ (2×11 L) and water (11 L). The wet toluene batch was then held for 36 hours while a second batch of equal size was processed. The wet toluene batches containing isopropyl ketone 12 were then combined and concentrated to approximately 2-3 volumes and the KF<200 ppm and were used directly in the next step. HPLC retention time of isopropyl ketone 12=8.1 min on 25 cm Zorbax SB C-18, using MeCN/0.1H₃PO₄, 1 mL/min. gradient elution: 70% MeCN for 5 minutes then to 90% MeCN at 10 minutes; 210 nm; hold for 5 minutes. ¹H NMR (400 MHz, CDCl₃) δ 7.85 (1H, br. s, Ar—H), 7.56 (1H, dd, 8.9 Hz, 1.0 Hz, Ar—H), 7.43 (1H, dd, 8 Hz, 1.0 Hz, Ar—H), 3.43 (1H, sep. 6.9 Hz, C—H), 1.21 (6H, d, 6.9 Hz, (CH₃)₂CH).

Step B: Synthesis of Fluoro Ketone 13. To a 100 L round bottomed flask fitted with a thermocouple, under an atmosphere of N₂ was added sequentially sodium amylate (3.0 kg, 27.3 mol) and DMF (11 L). The slurry was aged for 30 minutes until almost all of the base had dissolved. Then the solution was cooled to 10° C. and a −50 weight % solution of the isopropyl ketone 12 (10.8 kg, 50 weight %, 5.4 kg, 22 mol) was added over 55 minutes while maintaining the temperature between 15-20° C. via the controlled addition of the isopropyl ketone. Upon complete addition, the solution was aged for 30 minutes, cooled to 10° C. and treated with TBSCl (4.3 kg, 28.5 mol) over a 30 minute period while maintaining the temperature between 20-28° C. via controlled TBSCl addition. The reaction was aged for 30 minutes, then Select-Fluor™ (8.5 kg, 24 mol, Air Products) was then added over 1.5 hours while maintaining the reaction temperature 28-35° C. and the slurry aged for 1 hour. On complete conversion, water (18 L) was added, followed by toluene (12 L). The resulting biphasic mixture was transferred to a 100 L cylindrical vessel and aged for 10 minutes. The layers were separated and the organic phase was washed with additional water (9 L). The wet toluene solution containing fluoro ketone 13 was held for 36 hours while a second batch of equal size was processed. The combined toluene batches containing fluoro ketone 13 were then concentrated until the KF<200 ppm and about 50 weight %. The resulting toluene solution of fluoro ketone 13 was used directly in the next step. HPLC retention time fluoro ketone 13=8.5 minutes on 25 cm Zorbax SB C-18, using MeCN/0.1H₃PO₄, 1 mL/min. gradient elution: 70% MeCN for 5 minutes, then to 90% MeCN at 10 minutes, hold for 5 minutes at 210 nm.

IR νₘₐₓ/cm⁻¹ 3083 (C—H), 2989 (C—H), 2941 (C—H), 1696 (C═O), 1577 (C═C); ¹H NMR (500 MHz, CDCl₃) δ 8.02 (1H, s, ArH), 7.74 (1H, br d, J_{HF}=9.1 Hz, ArH), 7.65 (1H, dt, J_{HF}=7.66 Hz, J_{H,H}=2.0 Hz, ArH), 1.69 (6H, d, J_{H,F}=21.7 Hz, 2×CH₃); ¹³C NMR (125 MHz, CDCl₃) δ 198.2 (dd, J=27.1 Hz, 1.8 Hz, C═O), 162.2 (d, J=252.3 Hz, ArCF), 136.9 (dd, J=6.8, 4.3 Hz, ArCCO), 128.9 (dd, J=8.6, 3.1 Hz, ArCH), 123.5 (d, J=24.6 Hz, ArCH), 122.8 (dd, J=9.2, 1.2 Hz, ArCBr), 115.8 (dd, J=22.8, 9.2 Hz, ArCH), 99.9 (d, J=180.3 Hz, C(CH₃)₂), 25.5 (d, J=24.0 Hz, 2×CH₃); ¹⁹F NMR (470 MHz, CDCl₃) δ −109.96, Ar—F), −144.68 (C—F).

Step C: Synthesis of α,β-Unsaturated Ester 14. To a 50 L round bottomed flask fitted with a thermocouple, under an atmosphere of N₂, was added sequentially DMF (10 L), potassium carbonate (5.3 kg, 38.4 mol) and trimethylphosphonoacetate (5.3 kg, 29.1 mol). The slurry was heated to 70° C. and treated with the toluene solution of fluoro ketone 13 (9.9 kg of 50 weight % solution, 4.96 kg, 18.8 mol) over 46 minutes. The reaction was heated to 80-82° C. and aged at this temperature for 10 hours. Then the reaction was cooled to room temperature and aged overnight for 10 hours. Upon complete consumption of the fluoro ketone 13, the slurry was inverse quenched into a 100 L cylindrical vessel containing 2N citric acid (20 L) and toluene (10 L). The layers were mixed for 10 minutes before being separated. The toluene organic phase containing α,β-unsaturated ester 14 was washed with water (10 L), and used in the next step.

Step D: Synthesis of α,β-Unsaturated Acid 15. The toluene solution containing α,β-unsaturated ester 14 of Step C was charged to a 50 L round bottomed flask fitted with a thermocouple, under a N₂ atmosphere. Next, methanol (17 L) and 5N NaOH (8 L) were added. The mixture was heated to 45° C. and stirred rapidly for 3.5 hours. Then the reaction was cooled to 20° C. and transferred to a 100 L cyclindrical vessel containing heptane (15 L) and water (15 L). The resulting biphasic mixture was stirred rapidly for 10 minutes and the phases were separated. The aqueous phase was washed with heptane (10 L) and the layers were separated. The aqueous layer was charged into a 50 L round bottomed flask and acidified with 5N HCl until the pH=5. Next, α,β-unsaturated acid 15 seed was added (60 g) and the solution was slowly acidified to pH 2 over 1 hour via the dropwise addition of 5 N HCl. The solid α,β-unsaturated acid 15 was collected by filtration, washed with 1:1 methanol/water (15 L) and dried by pulling N₂ through the cake for 36 hours. A second batch on the same scale was processed to afford α,β-unsaturated acid 15 as an off white solid. HPLC retention time α,β-unsaturated acid 15=13.3 minutes on 25 cm Zorbax SB C-18, using MeCN/0.1H₃PO₄, 1 mL/min. gradient elution: 10% MeCN-50% MeCN over 0-5 minutes, then 50-90% MeCN over 5-20 minutes, and hold at 90% MeCN for additional 5 minutes at 210 nm. IRνₘₐₓ/cm⁻¹ (film) 3087 (O—H), 2986 (C—H), 2940 (C—H), 1703 (C═O), 1645 (C═C); ¹H NMR (500 MHz, CDCl₃) δ 7.25 (1H, dt, J_{H,F}=8.3 Hz, J_{H,H}=1.9 Hz, Ar—H), 7.06 (1H, s, Ar—H), 6.81 (1H, br. dt, J_{H,F}=8.7 Hz, J_{H,H}=1.6 Hz, Ar—H), 6.27 (1H, s, C═CH), 1.5 (6H, d, J_{H,F}=21.9 Hz); ¹³C NMR (125 MHz, CDCl₃) δ 170.0 (C═O), 162.0 (d, J=251.0 Hz, ArC-F), 159.7 (dd, J=19.1 Hz, J=1.8 Hz, C═CO₂H), 139.4 (dd, J=8.6 Hz, J=3.1, ArCC), 126.7 (d, J=3.1 Hz, ArCH), 122.1 (d, J=3.1 Hz, ArCBr), 118.6 (d, J=24 Hz, ArCH), 117.0 (d, =13.5 Hz, C═CCO₂H), 114.3 (d, J=22.8 Hz, ArCH), 95.3 (d, J=179.0 Hz, C—F), 26.4 (d, J=25.2 Hz, 2×CH₃); ¹⁹F NMR (470 MHz, CDCl₃) δ −110.4 (Ar—F), −138.09 (C—F).

Step E: Synthesis of α,β-Saturated Acid 16. The α,β-unsaturated acid 15 (2.2 kg, 7.2 mol) was weighed into a 4 L beaker and transferred into a poly jug. The beaker was then rinsed with MeOH (2 L) and the solution was transferred to the poly jug. Next, triethylamine (252 mL, 1.8 mol) was added and the funnel was rinsed down with the remaining MeOH (7 L). After rigorous mixing, the batch was transferred into a 5 gallon vessel followed by degassing of the solution. Then the catalyst was prepared as follows: the [Ru(cymene)Cl]₂ precursor (1.7 g, 0.0055 mol 0.076 mol %) and the (R)-1-[(S)-2-Di-2-furylphosphinogerrocenyl]ethyldi-tert.-butyl-phosphine (or SL-J212-1) ligand (3.1 g, 0.006 mol, 0.083 mol %) were charged into a 100 mL round bottomed flask under a N₂ atmosphere. Next, the solids were dissolved in 1:3 CH₂Cl₂/MeOH (21 mL) and stirred using a magnetic stir bar for 1 hour. After aging for 1 hour, the catalyst solution was transferred to a stainless steel reservoir using 20 mL of MeOH. An additional 40 mL of MeOH was transferred to the stainless steel reservoir for rinsing. Upon sealing the reservoirs, the solution was treated with H₂ under 200 psig at 50° C. for 10 hours to provide a methanolic solution of the α,β-saturated acid 16 in 92% ee, which was used in the next step.

Step F: Preparation of α,β-Saturated Ester 17. To a 100 L round bottom flask was added a methanolic solution of the α,β-saturated acid 16 (4.50 kg, 14.6 mol). The solution was cooled to −15° C., then acetyl chloride (2.2 kg, 2.0 L, 28.6 mol) was added dropwise over 30 minutes maintaining the temperature between −15° C. to −5° C. with a dry ice/acetone bath. The reaction was then warmed to room temperature and aged for 3 hours until complete by HPLC. The solution was transferred to a 100 L cylindrical vessel. Toluene (20 L) was added, followed by 7% aqueous NaCl (18 L). The resulting bi-phasic solution was heated to 30° C. and stirred for 10 minutes. Then the phases were separated, and the organic phase containing α,β-saturated ester 17 was washed with K₂HPO₄ solution (10 L). This process was repeated on a second 4.5 kg batch of α,β-saturated acid 16. The combined wet toluene batches containing α,β-saturated ester 17 were concentrated to about 2 volumes. HPLC retention time of α,β-saturated ester 17=7.5 minutes on ACE-111-15030 column using gradient elution: 0.1% aq. H₃PO₄/CH₃CN 60%-20% 0-6 minutes, then 20%-5% 6-8 minutes; 38° C., 220 nm. ¹H NMR (400 MHz, CDCl₃) δ 7.22 (1H, br. s, Ar—H), 7.15 (1H, dt, 6.1, 1.9 Hz, Ar—H), 6.94 (1H, br, d, 9.5 Hz, Ar—H), 3.58 (3H, s, OCH₃), 3.28 (1H, ddd, 20.6, 10.2, 4.5 Hz, CH), 2.88 (1H, dd, 16.2, 4.5, CH₂), 2.73 (1H, dd, 16.2, 10.2 Hz, CH₂), 1.38 (3H, d, 21.4 Hz, CH₃), 1.23 (3H, d, 21.6 Hz, CH₃).

Step G: Synthesis of β-carboxy ester 18. To a 75 L round bottom flask was added a toluene solution of the α,β-saturated ester 17 (3-4 volumes, 18 L total), followed by DMPU (1.7 kg, 13.3 mol). The solution was cooled to −43° C., then LiHMDS (12.2 L, 1.3 M, 15.9 mol) in THF was slowly added over 1 hour, while controlling the exotherm with a dry ice/acetone bath. Upon completion, the reaction cooled to −66° C. and dry CO₂ gas was slowly bubbled through the solution for 1 hour keeping the reaction temperature <−58 to −60° C. The reaction was allowed to re-cool to −66° C. and inverse quenched into water while maintaining the temperature of the aqueous mixture between 0-15° C. The organic phase was separated (aqueous pH=10-11). Toluene (18 L) was then added to the aqueous layer, followed by the slow addition of H₃PO₄ (85 weight %, 3.5 L) until the pH was <3. The aqueous phase was separated and the organic phase washed twice with 2.5N HCl (11 kg). An additional 4.1 kg of α,β-saturated ester 17 was processed using the above procedure. The combined toluene extracts containing β-carboxy ester 18 were then concentrated to about 3-4 volumes. HPLC retention time of β-carboxy ester 18=12.0 minutes on 4.6*150 mm, 5 μm diameter Zorbax Eclipse XD8-C8 column, using CH₃CN/0.1% H₃PO₄ in H₂O; 1 ml/min, 5 μl injection, 210 nm, gradient elution: t=0 minutes, 90% H₂O/CH₃CN; t=15 minutes, 10% H₂O/CH₃CN; and t=20 minutes, 10% H₂O/CH₃CN.

Step H: Synthesis of bromo diol 19. To a 100 L round bottom flask, under N₂ was added DME (23.9 kg), followed by NaBH₄ (2.2 kg, 59.2 mol.). The resulting slurry was cooled to −20° C. before adding bromine (1.4 L, 26.5 mol) via a dropping funnel over 2.5 hours while maintaining the temperature between −10° C.-20° C. Upon addition of the bromine, the solution was aged for 2 hours and then allowed to warm up to 10° C. The solution of β-carboxy ester 18 in DME/toluene (10.6 kg solution, estimated 4.4 kg, 12.0 mol) was then slowly added to the bromine solution over 2 hours, while cooling with an ice/water bath to maintain the reaction temperature ≦35° C. during addition. The reaction was then aged for 19 hours at room temperature, upon which time HPLC analysis indicated complete consumption of β-carboxy ester. The white heterogeneous mixture was inverse quenched into toluene (4 L) and 2N K₂CO₃ (38.6 kg) at 5° C. with cooling to control the exotherm. Additional toluene (7 L) was used to rinse the glassware. The combined toluene solutions were warmed up to 35° C. for 2 hours, then allowed to cool to room temperature and aged for 15 hours. The layers were separated (pH aqueous=11-12) and the organic layer was washed with 0.5 N HCl (9.4 kg; pH aqueous=0-1), then with 2N K₂CO₃ (22 kg with a 5 minute age; pH aqueous=13) and with 2N K₂CO₃ (9.1 kg with a 5 minute age, pH aqueous=13-14). The layers were then separated to give a wet DME/toluene layer containing bromo diol 19. A second batch of 4.4 kg of β-carboxy ester 18 was processed in the same way. The DME/toluene layers containing bromo diol 19 were combined and distilled to approximately 2 volumes. HPLC retention time of bromo diol 19=4.36 minutes on ACE-111-1503, gradient elution: 60%-5% 0.1% H₃PO₄, 220 nm. ¹H NMR (400 MHz, CDCl₃) δ 7.26-7.24 (1H, m, Ar—H), 7.17-7.14 (1H, m, Ar—H), 6.91 (1H, dt, 9.7, 1.7 Hz, Ar—H), 3.89 (1H, dd, 11.0, 6.0 Hz, CH), 3.81-3.69 (2H, m, 2×CH), 3.29 (1H, dd, 11.0, 7.3 Hz, CH), 3.26-3.20 (2H, m, CH, OH), 2.99 (1H, dd, 22.3, 8.3, CH), 2.32-2.25 (1H, m, OH), 1.37 (3H, d, 21.8 Hz, CH₃), 1.31 (3H, d, 22.3 Hz, CH₃).

Step I: Synthesis of cyano diol III. Crude bromo diol 19 (5 kg, 15.5 mol) in toluene (−20 weight %) was solvent switched into DMF (2.5 volumes, approximately 12.5 L), so that toluene was reduced to <5 LCAP by HPLC. To a slurry of Zn powder (615 g, 9.4 mol, <10 micron, Aldrich) and DMF (3 volumes, approximately 15 L), was added bromine (90 mL, 1.75 mol) slowly at room temperature under N₂. The mixture was agitated at room temperature for 20 minutes during which the color changed from orange to colorless. Then, Zn(CN)₂ (1.1 kg, 9.4 mol), the solution of bromo diol 19 (in 2.5 volume DMF, rinsed with 0.5 volumes of DMF), PPh₃ (485 g, 1.85 mol) and Pd(OAc)₂ (103 g, 0.46 mol) were added sequentially, and the resulting mixture was degassed for 30 minutes using a subsurface stream of nitrogen. The reaction mixture was then warmed to 80° C. under a nitrogen atmosphere, aged for 3 hours, then cooled to room temperature and aged overnight. The mixture was treated with 30% NH₄OH (0.84 volumes) and aged for 1 hour. The resulting slurry was filtered over Solka Floc™, and the bed was washed with IPAc (85 L). The filtrate was transferred to an extractor containing 10% NH₄OH (60 L). The organic solution was washed once with 5% NaCl (30 L) and water (30 L). The organic layer was solvent switched to toluene (˜9 L/kg cyano diol), at constant volume, at −40° C. until IPAc was reduced to <1 mol % (assay by GC). Heptane (5 L/kg cyanodiol) was added at ˜45° C. and the resulting slurry was allowed to cool to room temperature, and then to 0° C. The slurry was filtered and washed with 30:70 heptane:toluene (15 L) to give cyano diol III as a white solid (98 LCAP, 95% ee). HPLC retention time of cyano diol III=8.9 minutes on 4.6*250 mm, 5 μm diameter Waters Symmetry C18; eluant: CH₃CN/0.1% H₃PO₄ in H₂O; 1 ml/min, 5 μl injection, 210 nm, gradient elution: t=0 min, 65% H₂O/CH₃CN; t=4 min, 65% H₂O/CH₃CN; t=25 min, 20% H₂O/CH₃CN; t=26 min, 0% H₂O/CH₃CN; and t=30 min 0% H₂O/CH₃CN. α_{D}=+26.0° IR cm⁻¹ (film) 3395 (O—H), 2912 (C—H, 2233 (CN), 1593, 1439; ¹H NMR (400 MHz, CDCl₃) δ 7.37 (1H, t, J=1.4 Hz, Ar—H), 7.31-7.25 (2H, m, Ar—H), 3.98 (1H, dd, J=11.2, 5.3 Hz), 3.84 (1H, dd, J=11.0, 3.2 Hz), 3.76 (1H, ddd, J=11.2, 4.7, 1.0 Hz), 3.28 (1H, dd, J=11.0, 6.7 Hz), 3.20 (1H, dd, J=23.2, 8.1 Hz), 1.37 (3H, d, J=21.7 Hz), 1.36 (3H, d, J=22.2 Hz); ¹³C NMR (125 MHz, CDCl₃) δ 163.3, 160.8, 144.0, 143.9, 143.85, 129.4, 122.0, 121.7, 118.1, 117.8, 117.59, 117.56, 113.85, 113.75, 99.0, 97.31, 64.52, 64.47, 63.78, 63.76, 52.13, 52.11, 51.90, 51.89, 43.01, 43.00, 28.13, 27.89, 24.80, 24.55; ¹⁹F NMR (470 MHz, CDCl₃) δ −109.6 (Ar—F), −137.5 (C—F); m/z 269.1227; HRMS 269.1235.

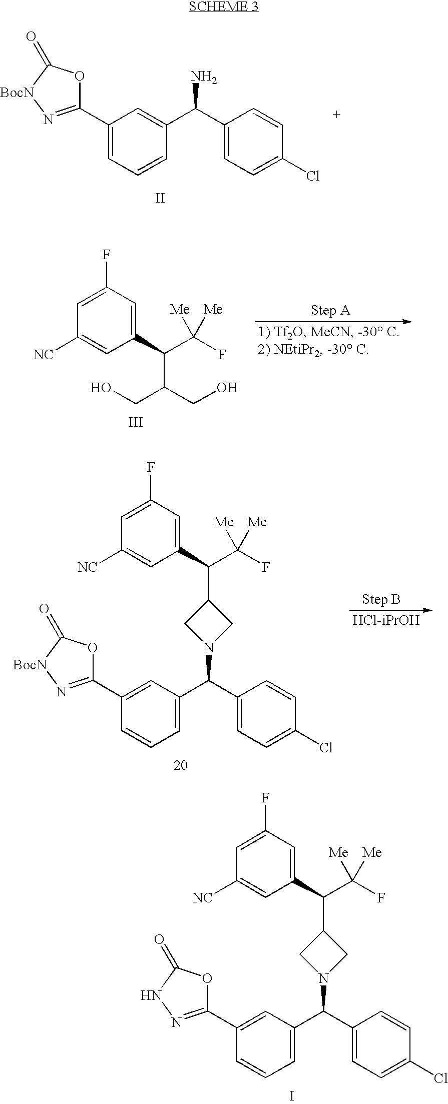

### Example 3

### Preparation of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I) Anhydrous Free Base Polymorphic Form I

Step A: Preparation of Azetidine 20 To a 75-L vessel equipped with thermocouple, addition funnel and overhead stirrer was charged cyanodiol III (0.94 kg, 3.48 mol) and acetonitrile (14 L) and the contents were cooled to −30° C. using acetone/dry ice. To the solution was slowly added triflic anhydride (1.29 L, 7.66 mol), followed by the slow addition of diisopropylethylamine (1.52 L, 2.5 eq). Both additions were performed while maintaining the internal temperature below −20° C. The reaction to form the bis-triflate was complete by HPLC after 1 hour at −30° C. Additional diisopropylethylamine (1.52 L, 2.5 eq) was added slowly, followed by the slow addition of benzyhydrylamine II (1.4 kg, 3.48 mol) as a solution in dichloromethane (5.6 L) via addition funnel, while maintaining the internal temperature below −20° C. The addition funnel was then rinsed with dichloromethane (0.7 L). The cooling bath was removed and the reaction mixture warmed to room temperature overnight. Then the reaction was then concentrated and solvent switched to IPAc using 28 L of IPAc and concentrated to a final volume of ˜10.5 L. The resulting slurry was diluted with heptane (5.6 L) to give ˜3:2 mixture of IPAc/heptane. After aging overnight at room temperature, the slurry was filtered to remove the diisopropylethylamine triflic acid salt, and the resulting salt cake was washed with a 3:2 mixture of IPAc/heptane (3×4.9 L). The filtrate was concentrated to ˜15 L and treated with Nuchar Aquaguard Powder™ (0.56 kg). The mixture was heated to 50° C. for 1 hour and then cooled to room temperature overnight. The resulting slurry was filtered through a pad of Solka Floc™ (1.4 kg), and the cake was washed with IPAc (5×2.8 L). The filtrate was transferred to a 50 L extractor and washed with GMP water (1×7 L). The organic layer was transferred to a 50 L vessel through a 1 micron in-line filter. The filtrate was concentrated to ˜10 L, then solvent switched to IPAc using 42 L of IPAc and concentrated to ˜6.4 L. The solution was cooled to room temperature overnight. Heptane (8.4 L) was slowly added over 1 hour followed by the addition of seed (7 g). After aging for 1 hour at room temperature, additional heptane (8.4 L) was slowly added over 2 hours, followed by a 1 hour age at room temperature. Heptane (4.2 L) was added over 1 hour at room temperature and the slurry was aged overnight at room temperature. This provided two types of crystals (fine and granular). Prior to filtration, the temperature was lowered to 0-5° C. for 2 hours. The slurry was filtered, and the resulting cake was slurry washed with 1:5 mixture of IPAc/heptane (1×2.8 L), followed by heptane (2×2.8 L). The cake was dried in the filter pot overnight under a nitrogen sweep and a vacuum pull from the bottom. The resulting azetidine 20 was obtained with a purity of 92 wt % and 97.8 LCAP. HPLC retention time of Azetidine 20=11.23 on Zorbax RX C8, Analytical 5.0 micron, 4.6 mm×250 mm, P.N.: 880967-901; temperature: 25° C.; detection at 210 nm; column flow is 1.0 ml/min; solvent A is acetonitrile; solvent B is H₂O buffered with 0.1% H₃PO₄, gradient elution: 0 minutes: 10% solvent A, 90% solvent B; 10 minutes: 90% solvent A, 10% solvent B; 15 minutes: 98% solvent A, 2% solvent B. ¹H (CDCl₃) δ 7.92 (t, J=1.6 Hz, 1H), 7.72 (dt, J=7.6, 1.6 Hz, 1H), 7.53 (d, J=7.6 Hz, 1H), 7.37 (d, J=7.6 Hz, 1H), 7.34 (m, 2H), 7.28-7.25 (om, 3H), 7.21 (ddd, J=7.6, 2.4-1.4 Hz, 1H), 7.16 (m, 1H), 4.27 (s, 1H), 3.61 (t, J=6.4 Hz, 1H), 3.16 (m, 1H) 3.05 (t, J=6.4 Hz, 1H), 2.91 (dd, J=19.1, 11.0 Hz, 1H), 2.85 (t, J=7.6 Hz, 1H), 2.27 (t, J=7.6 Hz, 1H), 1.66 (s, 9H), 1.27 (d, J=21.3 Hz, 3H), 1.19 (d, J=21.5 Hz, 3H). ¹⁹F (CDCl₃) δ −110.0, −144.3.

Step B: Preparation of Compound I To a 50 L vessel equipped with thermocouple, addition funnel and overhead stirrer was added azetidine 20 (1.85 assay kg, 2.91 mol), IPAc (5.6 L) and a solution of HCl in IPA (7.4 L, 4.55 M) with a mild endotherm (−6° C.). The reaction was complete by HPLC after aging overnight at room temperature. The reaction mixture was quenched by slowly transferring the batch to a 100 L extractor containing saturated NaHCO₃ (30.0 L) with a mild exotherm (˜5° C.). Additional saturated NaHCO₃ (5.0 L for a total of 35.0 L) was slowly added to adjust the pH to ˜7. Additional IPAc (13.0 L) was added, then the layers were separated and the organic layer was washed with GMP water (1×9.3 L). The organic layer was transferred to a 50 L vessel through a 1 micron in-line filter. The organic layer was concentrated to ˜10 L and the solvent was switched to IPAc using 18 L of IPAc, and concentrated to ˜18 L. The mixture was then treated with Nuchar Aquaguard Powder™ (0.45 kg), heated to 50° C. for 1 hour and then cooled to room temperature overnight. The resulting slurry was filtered through a pad of Solka Floc™ (1.85 kg), and the cake was washed with IPAc (4×7.0 L). The organic layer was transferred to a 50 L vessel through a 1 micron in-line filter, concentrated to ˜6 L and solvent switched to toluene using 28 L of toluene. The resulting solution was concentrated to ˜9.0 L, slowly heated to 45° C. and slowly treated with heptane (1.9 L over 1 hour while maintaining the internal temperature between 40-45° C. The batch was seeded (7 g) and aged for 1 hour at 45° C. Additional toluene (1.0 L) was added, then heptane (1.9 L) was slowly added over 2 hours at 45° C. while maintaining the reaction temperature between 40-45° C. After aging for 1 hour at 45° C., the reaction was allowed to cool to room temperature overnight. The slurry was filtered and the cake slurry was washed with 3:2 mixture of toluene/heptane (1×6.0 L), followed by heptane (2×6.0 L). The cake was then dried in the filter pot overnight under a nitrogen sweep and a vacuum pull from the bottom to give compound I with purity of 96.8 wt % and 98.6 LCAP. HPLC retention time of Compound I=9.54 on Zorbax RX C8, Analytical 5.0 micron, 4.6 mm×250 mm, P.N.: 880967-901; temperature: 25° C.; detection at 210 nm; column flow: 1.0 ml/min; solvent A: acetonitrile; solvent B: H₂O buffered with 0.1% H₃PO₄, gradient elution: 0 minutes: 10% solvent A, 90% solvent B; 10 minutes: 90% solvent A, 10% solvent B; 15 minutes: 98% solvent A, 2% solvent B. ¹H (CDCl₃) δ 10.85 (brs, 1H), 7.99 (t, J=1.6 Hz, 1H), 7.67 (dt, J=7.6, 1.6 Hz, 1H), 7.46 (d, J=7.6 Hz, 1H), 7.39-7.35 (om, 3H), 7.30-7.28 (om, 3H), 7.24-7.18 (om, 2H), 4.35 (s, 1H), 3.70 (t, J=6.8 Hz, 1H), 3.33 (m, 1H), 3.11 (td, J=7.6, 1.6 Hz, 1H), 3.01-2.93 (om, 2H), 2.40 (t, J=8.0 Hz, 1H), 1.27 (d, J=21.3 Hz, 3H), 1.22 (d, J=21.7 Hz, 3H). ¹⁹F (CDCl₃) 6-109.7, −142.7.

### Example 4

### Preparation of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I)

Free Base Toluene/Heptane Solvate Polymorphic Form I, Type B A portion of 3.36 kg of Compound I was taken up in 20 L of toluene and filtered though a 1 micron in-line filter. The slurry was heated to 53° C., and then cooled to 45° C. Then 1.01 kg of 10 weight % seed slurry (101 g of Compound I, media milled, in 6:4 toluene/heptane) was added to the mixture, followed by 6:4 toluene/heptane (779 g). The resulting slurry was aged for 1 hour. Heptane (10.1 L) was added over 4 hours 40 minutes at a rate of 38 mL/minute with a calibrated Encynova™ metered pump. The resulting slurry was cooled to room temperature and aged overnight. The mixture was filtered and washed with 6:4 toluene/heptane (14 L). The resulting cake was then washed with 28 L of heptane to give Compound I as the crystalline free base toluene/heptane solvate Form I, Type B.

The X-ray powder diffraction spectra of the free base toluene/heptane solvate polymorphic Form I, Type B of Compound I (FIG. 13) was generated on a Philips Analytical X'Pert PRO X-ray Diffraction System with PW3040/60 console. A PW3373/00 ceramic Cu LFF X-ray tube K-Alpha radiation was used as the source. The X-ray powder diffraction spectrum was recorded at ambient temperature (CuKα radiation, 2° to 40° (2θ), steps of 0.0167°, 5.08 sec per step). Cu K-α of wavelength 1.54178 Å was used for d-spacing calculation.

TABLE 1
Powder X-ray diffraction: free base toluene/heptane solvate
polymorphic Form I, Type B of Compound I

| 2θ (2 theta) (degrees) | d-spacing (Å) |
|---|---|
| 4.7 | 18.800 |
| 7.1 | 12.450 |
| 8.4 | 10.526 |
| 8.7 | 10.163 |
| 9.5 | 9.309 |
| 11.6 | 7.628 |
| 14.3 | 6.194 |
| 15.4 | 5.754 |
| 17.3 | 5.126 |

Although the anhydrous free base toluene/heptane solvate polymorphic Form I, Type B of Compound I is characterized by the complete group of angle 2 theta values listed in Table 1, all the values are not required for such identification. The free base toluene/heptane solvate polymorphic Form I, Type B of Compound I can be identified by the angle theta value of 4.7°. The free base toluene/heptane solvate polymorphic Form I, Type B of Compound I can be identified by any one of the following angle theta values, or any one of the following groups of angle theta values:

a) 4.7°;
b) 4.7° and 7.1°;
c) 4.7°, 7.1° and 8.4°;
d) 4.7°, 7.1°, 8.4° and 8.7°;
e) 4.7°, 7.1°, 8.4°, 8.7° and 9.5°;
f) 4.7°, 7.1°, 8.4°, 8.7°, 9.5° and 11.6°;
g) 4.7°, 7.1°, 8.4°, 8.7°, 9.5°, 11.6° and 14.3°;
h) 4.7°, 7.1°, 8.4°, 8.7°, 9.5°, 11.6°, 14.3° and 15.4°;
i) 4.7°, 7.1°, 8.4°, 8.7°, 9.5°, 11.6°, 14.3°, 15.4° and 17.3°.

The free base toluene/heptane solvate polymorphic Form I, Type B of Compound I can also be identified by one or more reflections at d-spacings of: 18.800, 12.450, 10.526, 10.163, 9.309, 7.628, 6.194, 5.754 and 5.126 Å from an x-ray powder diffraction pattern obtained using Cu radiation.

The thermogravimetric (TG) analysis curve for the free base toluene/heptane solvate polymorphic Form I Type B of Compound I (FIG. 14) was obtained under a nitrogen flow at a heating rate of 10° C./min in a Perkin Elmer TGA-7 instrument.

### Example 5

### Preparation of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I)

### Anhydrous Free Base Polymorphic Form I

The anhydrous free base Form I of compound I was obtained by drying the heptane/toluene solvate, which was prepared as described in Example 4. After drying the filter pot overnight, the white solid was transferred to the trays and dried in a vacuum at 25/75 Ton with nitrogen sweep to give Compound I as the anhydrous free base polymorphic Form I with 98.62 LCAP and >99% ee.

IR: νₘₐₓ (cm⁻¹) (film). ¹H NMR (400 MHz, CDCl₃) δ 11.5-11.8 (1H, bs, NH), 8.07 (1H, s, ArH), 7.66 (1H, d, J=7.7 Hz, ArH), 7.47 (1H, d, J=7.74, ArH) 7.39-1.21 (8H, m, ArH), 4.44 (1H, s, ArArCHN), 3.75 (1H, dd, J=6.8, 6.7 Hz, NCHH), 3.43-3.37 (1H, m, NCH₂CH), 3.16 (1H, dd, J=6.8, 6.7 Hz, NCHH), 3.06-2.99 (2H, m, NCHH, ArCHCH), 2.49 (1H, dd, J=8.2, 8.1 Hz, NCHH), 1.26 (3H, d, J=21.7 Hz, CH₃CF), 1.22 (3H, d, J=21.7 Hz, CH₃CF). ¹³C NMR (100 MHz, CDCl₃) δ 163.3, 160.8, 155.7, 154.9, 143.4, 143.3, 143.3, 141.6, 138.9, 133.7, 131.1, 129.5, 129.1, 128.8, 125.3, 125.0, 124.5, 121.5, 121.3, 118.1, 117.8, 117.6, 117.6, 113.7, 113.6, 97.2, 95.5, 77.6, 61.1, 59.4, 59.2, 58.2, 30.3, 25.9, 25.7, 25.3, 25.1. ¹⁹F NMR (376 MHz, CDCl₃) δ −109.7 (Ar—F), −142.3 (C—F).

The X-ray powder diffraction spectra of the anhydrous free base polymorphic Form I of Compound I (FIG. 1) was generated on a Philips Analytical X'Pert PRO X-ray Diffraction System with PW3040/60 console. A PW3373/00 ceramic Cu LFF X-ray tube K-Alpha radiation was used as the source. The X-ray powder diffraction spectrum was recorded at ambient temperature (CuKα radiation, 2° to 40° (2θ), steps of 0.0167°, 5.08 sec per step). Cu K-α of wavelength 1.54187 Å was used for d-spacing calculation.

TABLE 2
Powder X-ray diffraction: anhydrous free base polymorphic Form
I of Compound I

| 2θ (2 theta) (degrees) | d-spacing (Å) |
|---|---|
| 5.2 | 16.994 |
| 7.0 | 12.627 |
| 9.3 | 9.509 |
| 11.8 | 7.499 |
| 15.4 | 5.754 |
| 15.7 | 5.644 |
| 16.4 | 5.405 |
| 17.4 | 5.096 |
| 22.5 | 3.951 |

Although the anhydrous free base polymorphic Form I of Compound I is characterized by the complete group of angle 2 theta values listed in Table 2, all the values are not required for such identification. The anhydrous free base polymorphic Form I of Compound I can be identified by the angle theta value of 5.2°. The anhydrous free base polymorphic Form I of Compound I can be identified by any one of the following angle theta values, or any one of the following groups of angle theta values:

a) 5.2°;
b) 5.2° and 15.4°;
c) 5.2°, 15.4° and 9.3°;
d) 5.2°, 15.4°, 9.3° and 17.4°;
e) 5.2°, 15.4°, 9.3°, 17.4° and 7.0°;
f) 5.2°, 15.4°, 9.3°, 17.4°, 7.0° and 15.7°;
g) 5.2°, 15.4°, 9.3°, 17.4°, 7.0°, 15.7° and 22.5°;
h) 5.2° 15.4°, 9.3°, 17.4°, 7.0°, 15.7°, 22.5° and 11.8°;
i) 5.2°, 15.4°, 9.3°, 17.4°, 7.0°, 15.7°, 22.5°, 11.8° and 16.4°.

The anhydrous free base polymorphic Form I of Compound I can also be identified by one or more reflections at d-spacings of: 16.994, 5.754, 9.509, 5.096, 12.627, 5.644, 3.951, 7.499 and 5.405 Å from an x-ray powder diffraction pattern obtained using Cu radiation.

The thermogravimetric (TG) analysis curve for the anhydrous free base polymorphic Form I of Compound I (FIG. 2) was obtained under a nitrogen flow at a heating rate of 10° C./min in a Perkin Elmer TGA-7 instrument. The weight loss was <0.1% up to melting. The DSC curve for the anhydrous free base polymorphic Form I of Compound I (FIG. 3) was obtained on a TA Instruments DSC-2910 differential scanning calorimeter at a heating rate of 10° C./min under N₂ flow. The sample was heated in a closed pan. A single endotherm with onset at 157.8 C, peak at 163.6 C and as associated enthalpy of 44.6 J/g.

### Example 6

### Preparation of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I)

### Isopropyl Acetate/Methylcyclohexane Solvate Free Base Polymorphic Form I Type A

A portion of 300 mg of the free base of Compound I was dissolved in 0.25 mL of isopropylacetate. The mixture was heated to 70° C., and then 0.25 mL of methylcyclohexane was added. The mixture was cooled to 40° C., then seeded with the free base of Compound I. Solid began to slowly precipitate. The solution was cooled to room temperature and stirred for 30 minutes. Then 0.1 mL of isopropyl acetate and 0.5 mL methylcyclohexane was added. The resulting solid was filtered to give the methylcyclohexane/isopropyl acetate solvate of free base polymorphic Form I of Compound I.

The X-ray powder diffraction spectra of the isopropyl acetate/methylcyclohexane solvate free base polymorphic Form I, Type A of Compound I (FIG. 10) was generated on a Philips Analytical X'Pert PRO X-ray Diffraction System with PW3040/60 console. A PW3373/00 ceramic Cu LFF X-ray tube K-Alpha radiation was used as the source. The X-ray powder diffraction spectrum was recorded at ambient temperature (CuKα radiation, 2° to 40° (20), steps of 0.0167°, 5.08 sec per step). Cu K-α of wavelength 1.54178 Å was used for d-spacing calculation.

TABLE 3
Powder X-ray diffraction: isopropyl acetate/methylcyclohexane
solvate free base polymorphic Form I, Type A of Compound I

| 2θ (2 theta) (degrees) | d-spacing (Å) |
|---|---|
| 4.6 | 19.209 |
| 7.5 | 11.787 |
| 9.1 | 9.718 |
| 9.7 | 9.118 |
| 14.5 | 6.109 |
| 15.1 | 5.867 |
| 16.8 | 5.277 |
| 17.8 | 4.983 |
| 18.2 | 4.874 |

Although the isopropyl acetate/methylcyclohexane solvate free base polymorphic Form I, Type A of Compound I is characterized by the complete group of angle 2 theta values listed in Table 3, all the values are not required for such identification. The isopropyl acetate/methylcyclohexane solvate free base polymorphic Form I, Type A of Compound I can be identified by the angle theta value of 4.6°. The isopropyl acetate/methylcyclohexane solvate free base polymorphic Form I, Type A of Compound I can be identified by any one of the following angle theta values, or any one of the following groups of angle theta values:

a) 4.6°;
b) 4.6° and 7.5°;
c) 4.6°, 7.5° and 9.1°;
d) 4.6°, 7.5°, 9.1° and 9.7°;
e) 4.6°, 7.5°, 9.1°, 9.7° and 14.5°;
f) 4.6°, 7.5°, 9.1°, 14.5° and 15.1°;
g) 4.6°, 7.5°, 9.1°, 9.7°, 14.5°, 15.1° and 16.8°;
h) 4.6°, 7.5°, 9.1°, 9.7°, 14.5°, 15.1°, 16.8° and 17.8°;
i) 4.6°, 7.5°, 9.1°, 9.7°, 14.5°, 15.1°, 16.5°, 17.8° and 18.2°.

The isopropyl acetate/methylcyclohexane solvate free base polymorphic Form I, Type A of Compound I can also be identified by one or more reflections at d-spacings of: 19.209, 11.787, 9.718, 9.118, 6.109, 5.867, 5.277, 4.983 and 4.874 Å from an x-ray powder diffraction pattern obtained using Cu radiation.

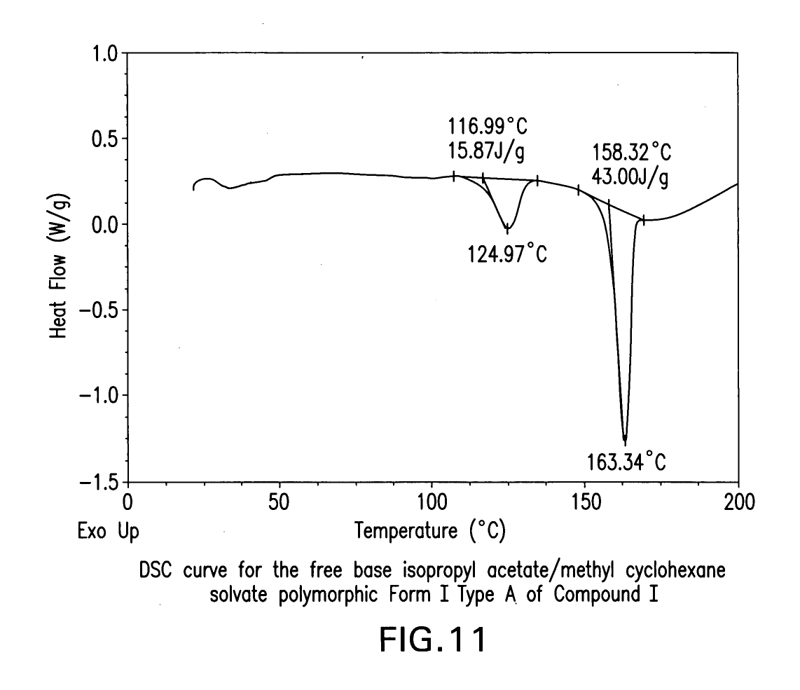

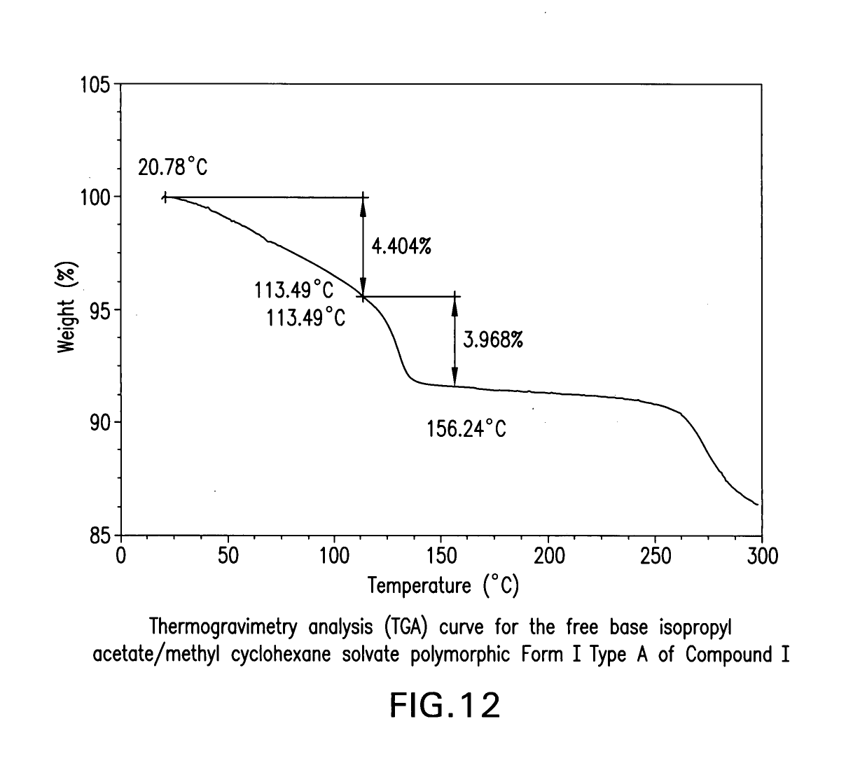

The thermogravimetric analysis (TGA) curve for the isopropyl acetate/methylcyclohexane solvate free base polymorphic Form I Type A of Compound I (FIG. 12) was obtained under a nitrogen flow at a heating rate of 10° C./min in a Perkin Elmer TGA-7 instrument. The DSC curve for the isopropyl acetate/methylcyclohexane solvate free base polymorphic Form I Type A of Compound I (FIG. 11) was obtained on a TA Instruments DSC-2910 differential scanning calorimeter at a heating rate of 10° C./min under N₂ flow.

### Example 7

### Preparation of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I)

### Anhydrous HCl Salt Polymorphic Form A

A portion of 0.5 g of the amorphous free base of Compound I was taken up in 2.5 mL of acetonitrile. Then 0.93 mL of 1.0M HCl/Et₂O were added dropwise, which resulted in a clear solution. After approximately 2 minutes, a white solid crystallized out of the solution. The mixture was stirred for an additional 10 minutes, then filtered. The resulting solid was dried on the frit under vacuum and a sweep of nitrogen to give the anhydrous HCl salt polymorphic Form A of Compound I.

Alternatively, the anhydrous HCl salt polymorphic Form A of Compound I may also be formed by a) crystallizing the HCl salt of Compound I from ethanol and drying; and b) drying the HCl salt polymorphic Form H of Compound I of Example 14.

The X-ray powder diffraction spectra for the anhydrous HCl salt polymorphic Form A of Compound I (FIG. 4) was generated on a Philips Analytical X'Pert PRO X-ray Diffraction System with PW3040/60 console. A PW3373/00 ceramic Cu LFF X-ray tube K-Alpha radiation was used as the source. The X-ray powder diffraction spectrum was recorded at ambient temperature (CuKα radiation, 2° to 40° (2θ), steps of 0.0167°, 5.08 sec per step). Cu K-α of wavelength 1.54178 Å was used for d-spacing calculation.

TABLE 4
Powder X-ray diffraction: HCl salt polymorphic Form A of Compound I

| 2θ (2 theta) (degrees) | d-spacing (Å) |
|---|---|
| 13.0 | 6.810 |
| 19.3 | 4.599 |
| 9.3 | 9.509 |
| 15.8 | 5.609 |
| 16.1 | 5.505 |
| 17.3 | 5.126 |
| 9.6 | 9.213 |
| 13.6 | 6.511 |
| 16.6 | 5.340 |

Although the anhydrous HCl salt polymorphic Form A of Compound I is characterized by the complete group of angle 2 theta values listed in Table 4, all the values are not required for such identification. The anhydrous HCl salt polymorphic Form A of Compound I can be identified by the angle theta value of 13.0°. The anhydrous HCl salt Form A of Compound I can be identified by any one of the following angle theta values, or any one of the following groups of angle theta values:

a) 13.0°;

b) 13.0° and 19.3°;

c) 13.0°, 19.3° and 15.8°;

d) 13.0°, 19.3°, 15.8° and 16.1°;

e) 13.0°, 19.3°, 15.8°, 16.1° and 17.3°;

f) 13.0°, 19.3°, 15.8°, 16.1°, 17.3° and 13.6°;

g) 13.0°, 19.3°, 15.8°, 16.1°, 17.3°, 13.6° and 9.3°;

h) 13.0°, 19.3°, 15.8°, 16.1°, 17.3°, 13.6°, 9.3° and 16.6°;

i) 13.0°, 19.3°, 15.8°, 16.1°, 17.3°, 13.6°, 9.3°, 16.6° and 9.6°.

The anhydrous HCl salt polymorphic Form A of Compound I can also be identified by one or more reflections at d-spacings of: 6.810, 4.599, 9.509, 5.609, 5.505, 5.126, 9.213, 6.511 and 5.340 Å from an x-ray powder diffraction pattern obtained using Cu radiation.

The anhydrous HCl salt polymorphic Form A of Compound I is also characterized by differential scanning calorimetry (DSC). The DSC curve of the anhydrous HCl salt polymorphic Form A of Compound I (FIG. 5) was obtained on a TA Instruments DSC-2910 differential scanning calorimeter at a heating rate of 10° C./min under N₂ flow. The sample was heated in a closed pan. A single endotherm with onset at 213.3 C is observed.

### Example 8

### Preparation of 3-[(1S)-1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I)

### Anhydrous HCl Salt Polymorphic Form B

A portion of 35 mL of isopropylacetate was added to 3.5 g of the amorphous free base of Compound I. Then, 1.44 mL of HCl in IPA (5-6M) was added. The reaction mixture was heated to 85-90° C., then sealed in a pressure tube. The reaction mixture was heated overnight on a heating mantle set to 150° C. The following morning the reaction mixture was cooled and filtered to give the HCl salt polymorphic Form B of Compound I.

Alternatively, the anhydrous HCl salt polymorphic Form B of Compound I may also be formed by: a) slurrying the HCl salt polymorphic Form C of Compound I of Example 9 in isopropyl acetate and drying; and b) slurrying the HCl salt polymorphic Form D of Compound I of Example 10 in ethanol with HCl salt polymorphic Form B Compound I seed and drying; c) slurrying the HCl salt polymorphic Form A of Compound I of Example 7 in isopropyl acetate and drying; and d) heating the HCl salt polymorphic Form A of Compound Ito a temperature greater than 215° C.

The X-ray powder diffraction spectra for the anhydrous HCl salt polymorphic Form B of Compound I (FIG. 6) was generated on a Philips Analytical X'Pert PRO X-ray Diffraction System with PW3040/60 console. A PW3373/00 ceramic Cu LFF X-ray tube K-Alpha radiation was used as the source. The X-ray powder diffraction spectrum was recorded at ambient temperature (CuKα radiation, 2° to 40° (2θ), steps of 0.0167°, 5.08 sec per step). Cu K-α of wavelength 1.54178 Å was used for d-spacing calculation.

TABLE 5
Powder X-ray diffraction: anhydrous HCl salt polymorphic
Form B of Compound I

| 2θ (2 theta) (degrees) | d-spacing (Å) |
|---|---|
| 17.9 | 4.955 |
| 6.1 | 14.488 |
| 15.3 | 5.791 |
| 8.1 | 10.915 |
| 10.9 | 8.117 |
| 14.2 | 6.237 |
| 19.4 | 4.575 |
| 21.5 | 4.133 |
| 24.9 | 3.576 |

Although the anhydrous HCl salt polymorphic Form B of Compound I is characterized by the complete group of angle 2 theta values listed in Table 5, all the values are not required for such identification. The anhydrous HCl salt polymorphic Form B of Compound I can be identified by the angle theta value of 17.9°. The anhydrous HCl salt polymorphic Form B of Compound I can be identified by any one of the following angle theta values, or any one of the following groups of angle theta values:
a) 17.9°;
b) 17.9° and 15.3°;
c) 17.9°, 15.3° and 19.4°;
d) 17.9°, 15.3°, 19.4° and 24.9°;
e) 17.9°, 15.3°, 19.4°, 24.9° and 6.1°;
f) 17.9°, 15.3°, 19.4°, 24.9°, 6.1° and 8.1°;
g) 17.9°, 15.3°, 19.4°, 24.9°, 6.1°, 8.1° and 21.5°;
h) 17.9°, 15.3°, 19.4°, 24.9°, 6.1°, 8.1°, 21.5° and 14.2°;
i) 17.9°, 15.3°, 19.4°, 24.9°, 6.1°, 8.1°, 21.5°, 14.2° and 10.9°.

The anhydrous HCl salt polymorphic Form B of Compound I can also be identified by one or more reflections at d-spacings of: 4.955, 14.488, 5.791, 10.915, 8.117, 6.237, 4.575, 4.133 and 3.576 Å from an x-ray powder diffraction pattern obtained using Cu radiation.

The anhydrous HCl salt polymorphic Form B of Compound I is also characterized by differential scanning calorimetry (DSC). The DSC curve for the anhydrous HCl salt polymorphic Form B of Compound I (FIG. 7) was obtained on a TA Instruments DSC-2910 differential scanning calorimeter at a heating rate of 10° C./min under N₂ flow. The sample was heated in a closed pan. A single endotherm with onset at 249.9 C is observed.

### Example 9

### Preparation of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I)

### HCl Salt Polymorphic Form C

A portion of 0.5 g of the amorphous free base of Compound I was taken up in 2.5 mL of isopropanol. Then 1.1 mole equivalents of 5-6N HCl/isopropyl alcohol were added dropwise. After stirring for 30 minutes, the resulting solid was filtered. The solid was dried on the frit under vacuum and a sweep of nitrogen to give the HCl salt polymorphic Form C of Compound I.

The X-ray powder diffraction spectra for the HCl salt polymorphic Form C of Compound I (FIG. 15) was generated on a Philips Analytical X'Pert PRO X-ray Diffraction System with PW3040/60 console. A PW3373/00 ceramic Cu LFF X-ray tube K-Alpha radiation was used as the source. The X-ray powder diffraction spectrum was recorded at ambient temperature (CuKα radiation, 2° to 40° (2θ), steps of 0.0167°, 5.08 sec per step). Cu K-α of wavelength 1.54178 Å was used for d-spacing calculation.

TABLE 6
Powder X-ray diffraction: HCl salt polymorphic Form C of Compound I

| 2θ (2 theta) (degrees) | d-spacing (Å) |
|---|---|
| 6.5 | 13.598 |
| 8.2 | 10.782 |
| 9.1 | 9.718 |
| 10 | 8.845 |
| 13.8 | 6.417 |
| 14.2 | 6.237 |
| 14.9 | 5.945 |
| 15.8 | 5.609 |
| 18.1 | 4.901 |

Although the HCl salt polymorphic Form C of Compound I is characterized by the complete group of angle 2 theta values listed in Table 6, all the values are not required for such identification. The HCl salt polymorphic Form C of Compound I can be identified by the angle theta value of 6.5°. The HCl salt Form C of Compound I can be identified by any one of the following angle theta values, or any one of the following groups of angle theta values:

j) 6.5°;

k) 6.5° and 8.2°;

1) 6.5°, 8.2° and 9.1°;

m) 6.5°, 8.2°, 9.1° and 10°;

n) 6.5°, 8.2°, 9.1°, 10° and 13.8°;

o) 6.5°, 8.2°, 9.1°, 10°, 13.8° and 14.2°;

p) 6.5°, 8.2°, 9.1°, 10°, 13.8°, 14.2° and 14.9°;

q) 6.5°, 8.2°, 9.1°, 10°, 13.8°, 14.2°, 14.9° and 15.8°;

r) 6.5°, 8.2°, 9.1°, 10°, 13.8°, 14.2°, 14.9°, 15.8° and 18.1°.

The HCl salt polymorphic Form C of Compound I can also be identified by one or more reflections at d-spacings of: 13.598, 10.782, 9.718, 8.845, 6.417, 6.237, 5.945, 5.609 and 4.901 Å from an x-ray powder diffraction pattern obtained using Cu radiation.

### Example 10

### Preparation of 3-[(1S)-1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I)

### HCl Salt Polymorphic Form D

The HCl salt polymorphic Form D of Compound I was prepared by drying crystalline HCl salt polymorphic Form F (hydrate) in a vacuum oven at 35° C.

The X-ray powder diffraction spectra for the HCl salt polymorphic Form D of Compound I (FIG. 16) was generated on a Philips Analytical X'Pert PRO X-ray Diffraction System with PW3040/60 console. A PW3373/00 ceramic Cu LFF X-ray tube K-Alpha radiation was used as the source. The X-ray powder diffraction spectrum was recorded at ambient temperature (CuKα radiation, 2° to 40° (2θ), steps of 0.0167°, 5.08 sec per step). Cu K-α of wavelength 1.54178 Å was used for d-spacing calculation.

TABLE 7
Powder X-ray diffraction: HCl salt polymorphic Form D of Compound I

| 2θ (2 theta) (degrees) | d-spacing (Å) |
|---|---|
| 7.2 | 12.277 |
| 12.3 | 7.196 |
| 6.2 | 14.255 |
| 11.1 | 7.971 |
| 13.8 | 6.417 |
| 18 | 4.928 |
| 8.8 | 10.048 |
| 22 | 4.040 |
| 24.2 | 3.678 |

Although the HCl salt polymorphic Form D of Compound I is characterized by the complete group of angle 2 theta values listed in Table 7, all the values are not required for such identification. The HCl salt polymorphic Form D of Compound I can be identified by the angle theta value of 7.2°. The HCl salt Form D of Compound I can be identified by any one of the following angle theta values, or any one of the following groups of angle theta values:

a) 7.2°;

b) 7.2° and 12.3°;

c) 7.2°, 12.3° and 6.2°;

d) 7.2°, 12.3°, 6.2° and 11.1°;

e) 7.2° 12.3°, 6.2°, 11.1° and 13.8°;

f) 7.2° 12.3°, 6.2°, 11.1°, 13.8° and 18°;

g) 7.2°, 12.3°, 6.2°, 11.1°, 13.8°, 18° and 8.8°;

h) 7.2°, 12.3°, 6.2°, 11.1°, 13.8°, 18°, 8.8° and 22°;

i) 7.2° 12.3°, 6.2°, 11.1°, 13.8°, 18°, 8.8°, 22° and 24.2°.

The HCl salt polymorphic Form D of Compound I can also be identified by one or more reflections at d-spacings of: 12.277, 7.196, 14.255, 7.971, 6.417, 4.928, 10.048, 4.040 and 3.678 Å from an x-ray powder diffraction pattern obtained using Cu radiation.

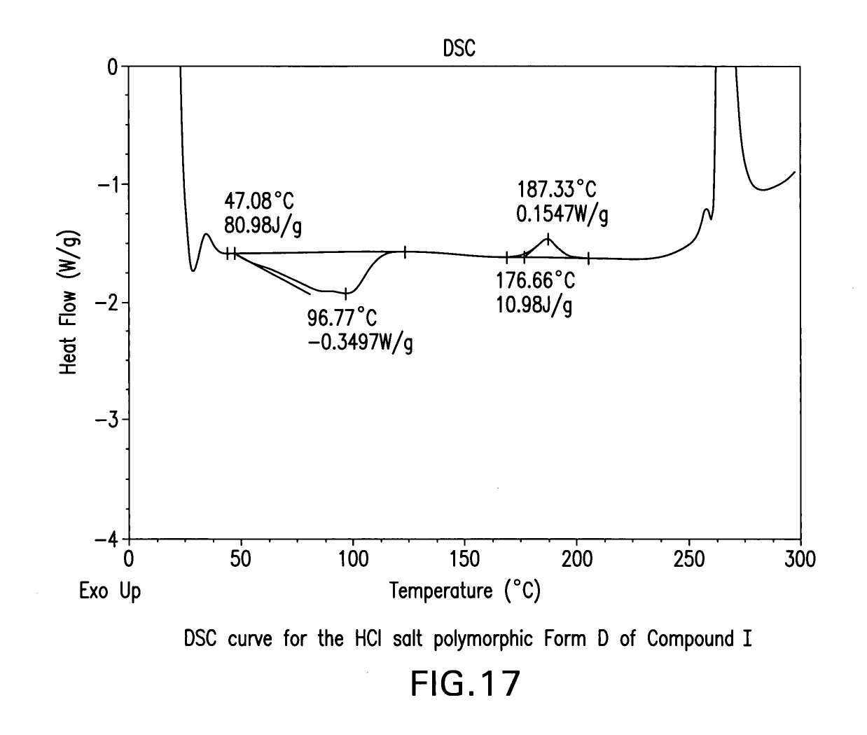

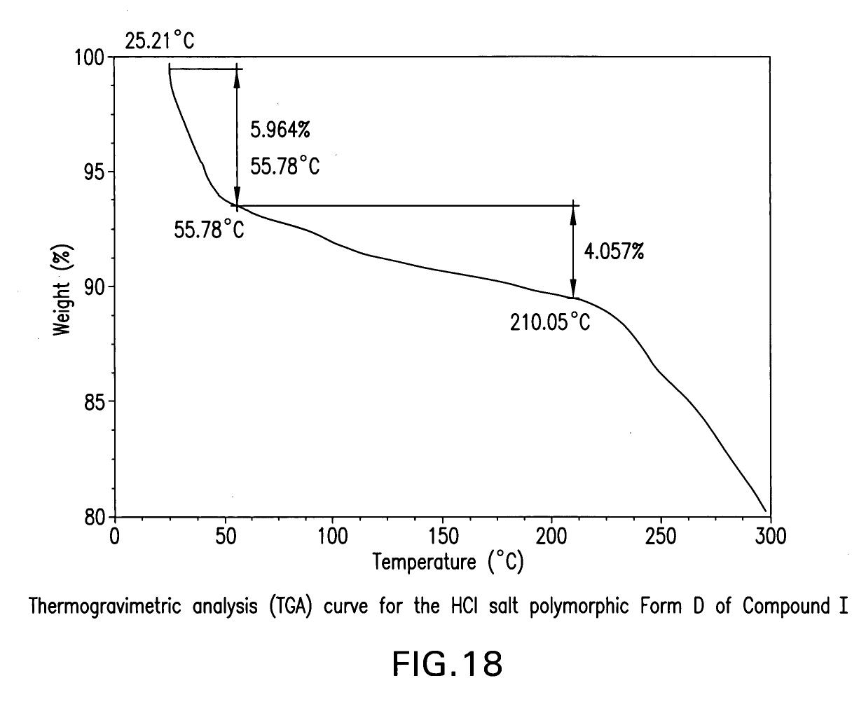

The thermogravimetric (TG) analysis curve for the HCl salt polymorphic Form D of Compound I (FIG. 18) was obtained under a nitrogen flow at a heating rate of 10° C./min in a Perkin Elmer TGA-7 instrument. The DSC curve for the HCl salt polymorphic Form D of Compound I (FIG. 17) was obtained on a TA Instruments DSC-2910 differential scanning calorimeter at a heating rate of 10° C./min under N₂ flow.

### Example 11

### Preparation of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I)

### Anhydrous HCl Salt Polymorphic Form E

The anhydrous HCl salt polymorphic Form E was prepared by drying HCl salt polymorphic Form D to a temperature>185° C.

The X-ray powder diffraction spectra for the anhydrous HCl salt polymorphic Form E of Compound I (FIG. 19) was generated on a Philips Analytical X'Pert PRO X-ray Diffraction System with PW3040/60 console. A PW3373/00 ceramic Cu LFF X-ray tube K-Alpha radiation was used as the source. The X-ray powder diffraction spectrum was recorded at ambient temperature (CuKα radiation, 2° to 40° (2θ), steps of 0.0167°, 5.08 sec per step). Cu K-α of wavelength 1.54178 Å was used for d-spacing calculation.

TABLE 8
Powder X-ray diffraction: HCl salt polymorphic Form E of Compound I

| 2θ (2 theta) (degrees) | d-spacing (Å) |
|---|---|
| 7.4 | 11.946 |
| 8.2 | 10.782 |
| 8.9 | 9.936 |
| 10.2 | 8.672 |
| 12.8 | 6.916 |
| 14.3 | 6.194 |
| 14.8 | 5.985 |
| 15.4 | 5.754 |
| 19.5 | 4.552 |

Although the anhydrous HCl salt polymorphic Form E of Compound I is characterized by the complete group of angle 2 theta values listed in Table 8, all the values are not required for such identification. The anhydrous HCl salt polymorphic Form E of Compound I can be identified by the angle theta value of 7.4°. The anhydrous HCl salt Form E of Compound I can be identified by any one of the following angle theta values, or any one of the following groups of angle theta values:

a) 7.4°;

b) 7.4° and 8.2°;

c) 7.4°, 8.2° and 8.9°;

d) 7.4°, 8.2°, 8.9° and 10.2°;

e) 7.4°, 8.2°, 8.9°, 10.2° and 12.8°;

f) 7.4°, 8.2°, 8.9°, 10.2°, 12.8° and 14.3°;

g) 7.4°, 8.2°, 8.9°, 10.2°, 12.8°, 14.3° and 14.8°;

h) 7.4°, 8.2°, 8.9°, 10.2°, 12.8°, 14.3°, 14.8° and 15.4°;

i) 7.4°, 8.2°, 8.9°, 10.2°, 12.8°, 14.3°, 14.8°, 15.4° and 19.5°.

The anhydrous HCl salt polymorphic Form E of Compound I can also be identified by one or more reflections at d-spacings of: 11.946, 10.782, 9.936, 8.672, 6.916, 6.194, 5.985, 5.754 and 4.552 Å from an x-ray powder diffraction pattern obtained using Cu radiation.

### Example 12

### Preparation of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I)

### HCl Salt Polymorphic Form F Hydrate

A portion of 2.5 g of the free base of Compound I was dissolved in 10 mL of ethanol. Then 0.685 g of the solution was charged to a vessel, followed by 2.5 mL of ethanol. Then 0.75 mL of 0.411 M aqueous HCl solution was added to the vessel. 1% seed was added and the slurry was aged for 1 hour. Then the remaining 11.3 mL of the free base solution and 3.1 mL of a 1.4 M aqueous HCl solution were added over 8 hours. The slurry was then filtered and washed with 1:1 ethanol/water to give the hydrate HCl salt polymorphic Form F.

The X-ray powder diffraction spectra for the HCl salt polymorphic Form F hydrate of Compound I (FIG. 20) was generated on a Philips Analytical X'Pert PRO X-ray Diffraction System with PW3040/60 console. A PW3373/00 ceramic Cu LFF X-ray tube K-Alpha radiation was used as the source. The X-ray powder diffraction spectrum was recorded at ambient temperature (CuKα radiation, 2° to 40° (2θ), steps of 0.0167°, 5.08 sec per step). Cu K-α of wavelength 1.54178 Å was used for d-spacing calculation.

TABLE 9
Powder X-ray diffraction: HCl salt polymorphic Form F hydrate
of Compound I

| 2θ (2 theta) (degrees) | d-spacing (Å) |
|---|---|
| 6.1 | 14.488 |
| 6.4 | 13.810 |
| 7.3 | 12.109 |
| 11 | 8.043 |
| 11.5 | 7.694 |
| 12 | 7.375 |
| 16.2 | 5.471 |
| 18 | 4.928 |
| 19 | 4.671 |

Although the HCl salt polymorphic Form F hydrate of Compound I is characterized by the complete group of angle 2 theta values listed in Table 9, all the values are not required for such identification. The HCl salt polymorphic Form F hydrate of Compound I can be identified by the angle theta value of 6.1°. The HCl salt Form F hydrate of Compound I can be identified by any one of the following angle theta values, or any one of the following groups of angle theta values:

a) 6.1°;

b) 6.1° and 6.4°;

c) 6.1°, 6.4° and 7.3°;

d) 6.1°, 6.4°, 7.3° and 11°;

e) 6.1°, 6.4°, 7.3°, 11° and 11.5°;

f) 6.1°, 6.4°, 7.3°, 11°, 11.5° and 12°;

g) 6.1°, 6.4°, 7.3°, 11°, 11.5°, 12° and 16.2°;

h) 6.1°, 6.4°, 7.3°, 11°, 11.5°, 12°, 16.2° and 18°;

i) 6.1°, 6.4°, 7.3°, 11°, 11.5°, 12°, 16.2°, 18° and 19°.

The HCl salt polymorphic Form F hydrate of Compound I can also be identified by one or more reflections at d-spacings of: 14.488, 13.810, 12.109, 8.043, 7.694, 7.375, 5.471, 4.928 and 4.671 Å from an x-ray powder diffraction pattern obtained using Cu radiation.

### Example 13

### Preparation of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I)

### Anhydrous HCl Salt Polymorphic Form G

A portion of 2.0 g of the crystalline free base of Compound I was taken up in 3.7 mL of ethanol. Then 0.05 mL of 1.25 M HCl in ethanol was added, followed by 80 mg of Form G seed. A portion of 3.2 mL of 1.25 M HCl in ethanol was then slowly added at a rate of 0.2 mL/hour. The resulting slurry was filtered, washed with 6 mL of ethanol, and dried on the fit under vacuum and a nitrogen sweep to give the anhydrous HCl salt polymorphic Form G of Compound I.

Alternatively, the anhydrous HCl salt polymorphic Form G of Compound I may also be formed by: a) slurrying the HCl salt polymorphic Form D of Compound I of Example 10 with HCl salt polymorphic Form G of Compound I; b) slurrying the HCl salt polymorphic Form B of Compound I of Example 8 in ethanol with HCl salt polymorphic Form G Compound I seed; c) slurrying the HCl salt polymorphic Form B of Compound I of Example 8 in 25% water/ethanol; and d) slurrying the HCl salt polymorphic Form A of Compound I of Example 7 in ethanol with HCl salt polymorphic Form G of Compound I.

The X-ray powder diffraction spectra for the anhydrous HCl salt polymorphic Form G of Compound I (FIG. 8) was generated on a Philips Analytical X'Pert PRO X-ray Diffraction System with PW3040/60 console. A PW3373/00 ceramic Cu LFF X-ray tube K-Alpha radiation was used as the source. The X-ray powder diffraction spectrum was recorded at ambient temperature (CuKα radiation, 2° to 40° (2θ), steps of 0.0167°, 5.08 sec per step). Cu K-α of wavelength 1.54178 Å was used for d-spacing calculation.

TABLE 10
Powder X-ray diffraction: anhydrous HCl salt polymorphic Form
G of Compound I

| 2θ (2 theta) (degrees) | d-spacing (Å) |
|---|---|
| 8.2 | 10.782 |
| 20.8 | 4.270 |
| 10.4 | 8.506 |
| 12.6 | 7.025 |
| 12.1 | 7.314 |
| 13.3 | 6.657 |
| 14.1 | 6.281 |
| 16.8 | 5.277 |
| 21.6 | 4.114 |

Although the anhydrous HCl salt polymorphic Form G of Compound I is characterized by the complete group of angle 2 theta values listed in Table 10, all the values are not required for such identification. The anhydrous HCl salt polymorphic Form G of Compound I can be identified by the angle theta value of 8.2°. The anhydrous HCl salt polymorphic Form G of Compound I can be identified by any one of the following angle theta values, or any one of the following groups of angle theta values:

a) 8.2°;

b) 8.2° and 20.8°;

c) 8.2°, 20.8° and 16.8°;

d) 8.2°, 20.8°, 16.8° and 21.6°;

e) 8.2°, 20.8°, 16.8°, 21.6° and 12.6°;

f) 8.2°, 20.8°, 16.8°, 21.6°, 12.6° and 10.4°;

g) 8.2°, 20.8°, 16.8°, 21.6°, 12.6°, 10.4° and 12.1°;

h) 8.2°, 20.8°, 16.8°, 21.6°, 12.6°, 10.4°, 12.1° and 14.1°;

i) 8.2°, 20.8°, 16.8°, 21.6°, 12.6°, 10.4°, 12.1°, 14.1° and 13.3°.

The anhydrous HCl salt polymorphic Form G of Compound I can also be identified by one or more reflections at d-spacings of: 10.782, 4.270, 8.506, 7.025, 7.314, 6.657, 6.281, 5.277 and 4.114 Å from an x-ray powder diffraction pattern obtained using Cu radiation.

The anhydrous HCl salt polymorphic Form G of Compound I is also characterized by differential scanning calorimetry (DSC). The DSC curve for the anhydrous HCl salt polymorphic Form G of Compound I (FIG. 9) was obtained on a TA Instruments DSC-2910 differential scanning calorimeter at a heating rate of 10° C./min under N₂ flow. The sample was heated in a closed pan. A single endotherm with onset at 267.3 C is observed.

### Example 14

### Preparation of 3-[(1S)-1-(1-{(S)-(4-chlorophenyl)[3-(5-oxo-4,5-dihydro-1,3,4-oxadiazol-2-yl)phenyl]methyl}azetidin-3-yl)-2-fluoro-2-methylpropyl]-5-fluorobenzonitrile (Compound I)

### HCl Salt Polymorphic Form H

A portion of 2.0 g of the crystalline free base of Compound I was taken up in 3.7 mL of ethanol. Then 0.05 mL of 1.25 M HCl in ethanol was added, followed by 5 mg of Form G seed. A portion of 3.2 mL of 1.25 M HCl in ethanol was then slowly added at a rate of 0.2 mL/hour. The resulting slurry was filtered, washed with 6 mL of ethanol and not dried.

The X-ray powder diffraction spectra for the HCl salt polymorphic Form H of Compound I (FIG. 21) was generated on a Philips Analytical X'Pert PRO X-ray Diffraction System with PW3040/60 console. A PW3373/00 ceramic Cu LFF X-ray tube K-Alpha radiation was used as the source. The X-ray powder diffraction spectrum was recorded at ambient temperature (CuKa radiation, 2° to 40° (2θ), steps of 0.0167°, 5.08 sec per step). Cu K-α of wavelength 1.54178 Å was used for d-spacing calculation.

TABLE 11
Powder X-ray diffraction: HCl salt polymorphic Form H of Compound I

| 2θ (2 theta) (degrees) | d-spacing (Å) |
|---|---|
| 9.1 | 9.718 |
| 9.4 | 9.408 |
| 12.8 | 6.916 |
| 13.4 | 6.607 |
| 14.3 | 6.194 |
| 14.9 | 5.945 |
| 15.3 | 5.791 |
| 16.9 | 5.246 |
| 18.2 | 4.874 |

Although the HCl salt polymorphic Form H of Compound I is characterized by the complete group of angle 2 theta values listed in Table 11, all the values are not required for such identification. The HCl salt polymorphic Form H of Compound I can be identified by the angle theta value of 9.1°. The HCl salt Form H of Compound I can be identified by any one of the following angle theta values, or any one of the following groups of angle theta values:

a) 9.1°;

b) 9.1° and 9.4°;

c) 9.1°, 9.4° and 12.8°;

d) 9.1°, 9.4°, 12.8° and 13.4°;

e) 9.1°, 9.4°, 12.8°, 13.4° and 14.3°;

f) 9.1°, 9.4°, 12.8°, 13.4°, 14.3° and 14.9°;

g) 9.1°, 9.4°, 12.8°, 13.4°, 14.3°, 14.9° and 15.3°;

h) 9.1°, 9.4°, 12.8°, 13.4°, 14.3°, 14.9°, 15.3° and 16.9°;

i) 9.1°, 9.4°, 12.8°, 13.4°, 14.3°, 14.9°, 15.3°, 16.9° and 18.2°.

The HCl salt polymorphic Form C of Compound I can also be identified by one or more reflections at d-spacings of: 9.718, 9.408, 6.916, 6.607, 6.194, 5.945, 5.791, 5.246 and 4.874 Å from an x-ray powder diffraction pattern obtained using Cu radiation.

1. A process for preparing a compound of formula 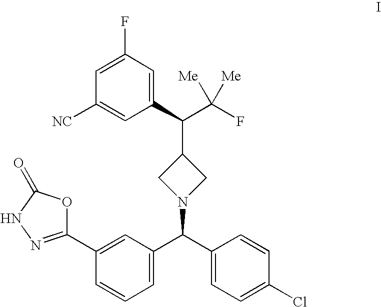
or a salt, hydrate, solvate or polymorph thereof,
comprising the steps of:
(A) removing the protecting group P of the compound of formula 20 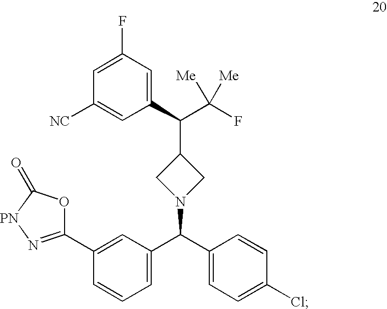
and
(B) isolating the resulting product.

2. The process of claim 1 wherein the protecting group P is CBZ, and the CBZ protecting group is removed by hydrogenation.

3. The process of claim 1 wherein the protecting group P is Boc, and the Boc protecting group is removed by treatment with an acid.

4. The process of claim 1 further comprising isolating the compound of formula I by crystallizing from toluene/heptane.

5. The process of claim 1 wherein the salt of the compound of formula I is the hydrochloric acid salt.

6. The process of claim 1 wherein the polymorph of the isolated compound of formula I is selected from the group consisting of:
(1) anhydrous free base polymorphic Form I of Compound I characterized by the X-ray powder diffraction pattern of FIG. 1;
(2) free base toluene/heptane solvate polymorphic Form I Type B of Compound I characterized by the X-ray powder diffraction pattern of FIG. 13;
(4) free base isopropyl acetate/methyl cyclohexane solvate polymorphic Form I Type A of Compound I characterized by the X-ray powder diffraction pattern of FIG. 10;
(5) anhydrous HCl salt polymorphic Form A of Compound I characterized by the X-ray powder diffraction pattern of FIG. 4;
(6) anhydrous HCl salt polymorphic Form B of Compound I characterized by the X-ray powder diffraction pattern of FIG. 6;
(7) HCl salt polymorphic Form C of Compound I characterized by the X-ray powder diffraction pattern of FIG. 15;
(8) HCl salt polymorphic Form D of Compound I characterized by the X-ray powder diffraction pattern of FIG. 16;
(9) anhydrous HCl salt polymorphic Form E of Compound I characterized by the X-ray powder diffraction pattern of FIG. 19;
(10) HCl salt polymorphic Form F hydrate of Compound I characterized by the X-ray powder diffraction pattern of FIG. 20;
(11) anhydrous HCl salt polymorphic Form G of Compound I characterized by the X-ray powder diffraction pattern of FIG. 8; and
(12) HCl salt polymorphic Form H of Compound I characterized by the X-ray powder diffraction pattern of FIG. 21.

7. A compound which is the anhydrous free base polymorphic Form T of Compound I:

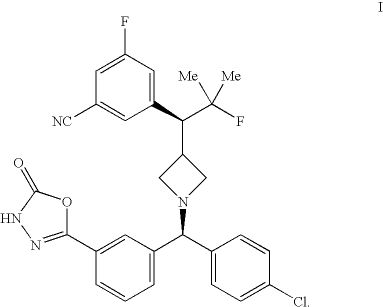

8. The anhydrous free base polymorphic Form 1 of Compound I of claim 7 characterized by the X-ray powder diffraction pattern of FIG. 1.

9. The compound of claim 7 having an X-ray powder diffraction pattern obtained using Cu radiation containing an angle 2 theta value of 5.2°-28.5°.

10. The compound of claim 7 having an X-ray powder diffraction pattern obtained using Cu radiation containing an angle 2 theta value of 5.2°.

11. The compound of claim 7 having an X-ray powder diffraction pattern obtained using Cu radiation containing the following angle 2 theta values: 5.2° and 7.0°.

12. The compound of claim 7 having an X-ray powder diffraction pattern obtained using Cu radiation containing the following angle 2 theta values: 5.2°, and 7.0°, and at least one angle theta value selected from the group consisting of: 9.3°, 11.8°, 15.4°, 15.7°, 16.4°, 17.4° and 22.5°.

13. The compound of claim 7 having an X-ray powder diffraction pattern obtained using Cu radiation characterized by a reflection at a d-spacing of 16.99 Å.

14. The compound of claim 7 having an X-ray powder diffraction pattern obtained using Cu radiation characterized by a reflection at a d-spacing of 16.99 Å, and at least one reflection at a d-spacing selected from the group consisting of: 12.63 Å, 9.51 Å, 7.5 Å, 5.75 Å, 5.64 Å, 5.40 Å, 5.09 Å and 3.95 Å.

15. The compound of claim 7 having a differential scanning calorimetry peak melting temperature of about 163.57° C.

16. A pharmaceutical composition comprising a therapeutically effective amount of the anhydrous free base polymorphic Form I of Compound T of claim 7, and a pharmaceutically acceptable carrier.

17. A method of treating obesity, diabetes, Alzheimer's Disease, or an obesity-related disorder in a subject in need thereof comprising administering to the subject a therapeutically effective amount of the anhydrous free base polymorphic Form I of Compound of claim 7.

18. (canceled)

19. A compound of formula II 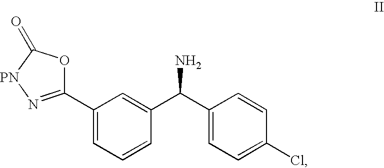
wherein P is Boc or CBZ, or salt, hydrate, or solvate thereof.

20. A compound of formula III: 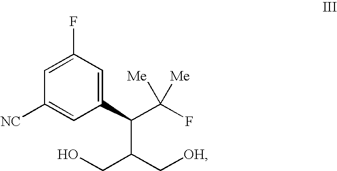
or a salt, hydrate or solvate thereof.

21. The process of claim 1 further comprising preparing a compound of formula 20, wherein P is a protecting group, 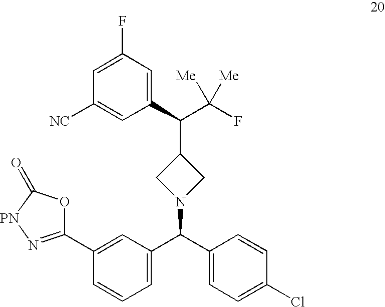
comprising the steps of coupling a compound of formula II wherein P is a protecting group, or a salt thereof, 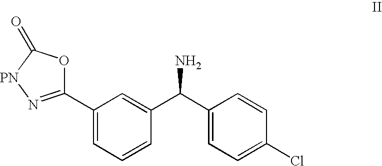
with a compound of formula III, 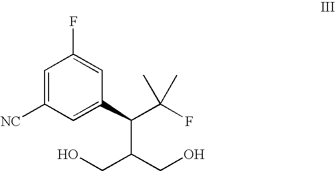
after converting the alcohol groups of compound III into leaving groups, followed by treatment with a hindered amine base.

22. The process of claim 21 wherein the leaving groups are triflates; compound III is treated with triflic anhydride to form a di-triflate intermediate; and the hindered amine base is diisopropyl ethyl amine.

23. The process of claim 21 further comprising preparing a compound of formula II wherein P is a protecting group, or a salt thereof, 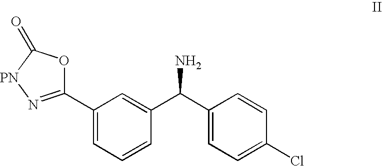
comprising the steps of:
(A) preparing a hydrazide of formula 3 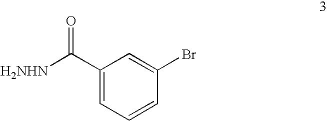
by treatment of a compound of formula 1 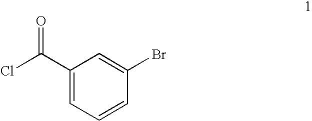
with a base, followed by treatment with hydrazine;
(B) forming an oxadiazole of formula 4 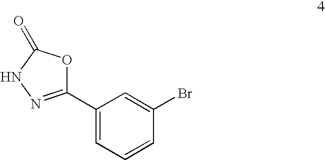
by treating the hydrazide of formula 3 with a coupling agent;
(C) preparing an aldehyde of formula 5 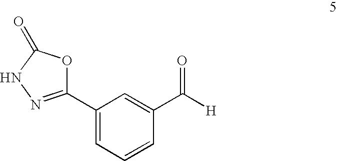
by treatment of the oxadiazole of formula 4 with an alkyl magnesium compound, followed by treatment with an alkyl lithium compound and DMF;
(D) preparing a N-tert-butyl sulfinyl imine of formula 6 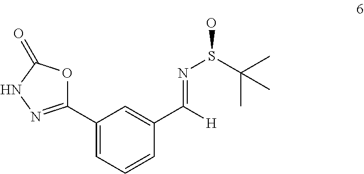
by treating the aldehyde of formula 5 with (S)-tent-butyl sulfinamide in the presence of a catalyst;
(E) forming a protected oxadiazole compound of formula 7, wherein P is a protecting group, 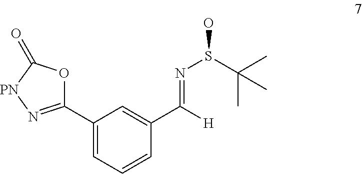
by adding a protecting group P to the oxadiazole nitrogen of the N-tert-butyl sulfinyl imine of formula 6;
(F) forming a N-tert-butyl sulfinyl amine of formula 8, wherein P is a protecting group, 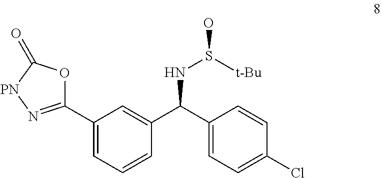
by treating the protected oxadiazole compound of formula 7 with boroxine 10 in the presence of a rhodium catalyst and a ligand; and
(G) forming a compound of formula II, wherein P is a protecting group, 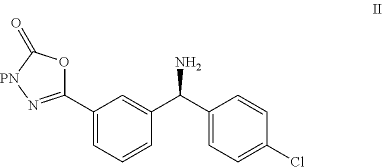
by cleaving the tent-butyl sulfoxide group of the N-tert-butyl sulfinyl amine of formula 8.

24. The process of claim 21 further comprising preparing a compound of formula III, or a salt thereof, 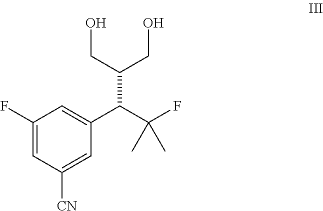
comprising the steps of:
(A) preparing a compound of formula 12: 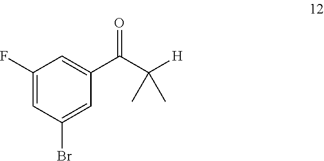
by treatment of a compound of formula 11 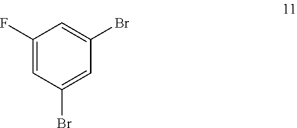
with a Grignard reagent, followed by treatment with isobutyryl chloride;
(B) forming a fluoro ketone compound of formula 13: 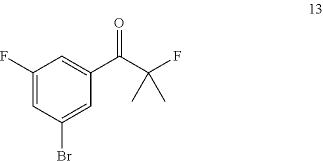
by fluorinating the compound of formula 12 by treatment with a fluorine source, and a base in the presence of a silyl halide or silyl triflate;
(C) preparing a compound of formula 14: 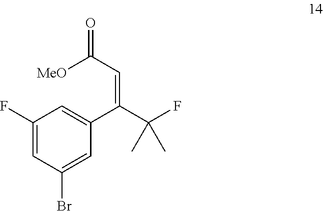
by treating the compound of formula 13 with trimethylphosphonoacetate in the presence of a base;
(D) preparing a compound of formula 15: 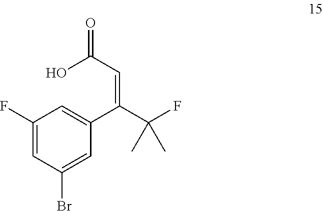
by hydrolyzing the ester of the compound of formula 14;
(E) forming a compound of formula 16: 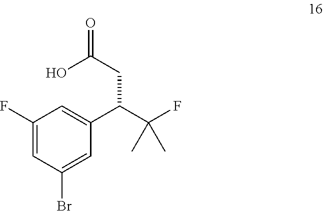
by reducing the double bond of compound of formula 15;
(F) forming a compound of formula 17: 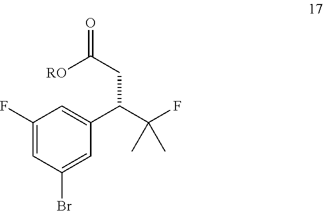
wherein R=C₁₋₃alkyl, by esterification of the compound of formula 16;
(G) forming a compound of formula 18: 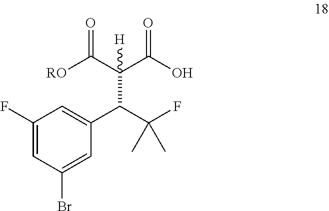
wherein R=C₁₋₃alkyl, by carboxylation of the compound of formula 17;
(H) forming a compound of formula 19: 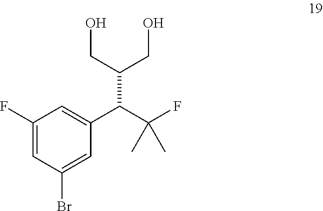
by reducing the compound of formula 18; and
(I) forming a compound of formula III 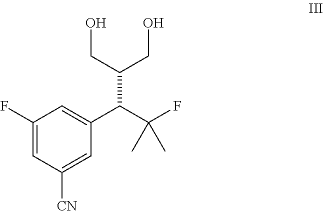
by cyanating the compound of formula 19.
